(19) United States
(12) Patent Application Publication
(10) Pub. No.: US 2025/0109177 A1
(43) Pub. Date: Apr. 3, 2025

(54) EVOLVED PROTEIN DEGRONS

(71) Applicants: The Broad Institute, Inc., Cambridge, MA (US); President and Fellows of Harvard College, Cambridge, MA (US); The Brigham and Women's Hospital, Inc., Boston, MA (US)

(72) Inventors: David R. Liu, Cambridge, MA (US); Amit Choudhary, Boston, MA (US); Jaron August McClure Mercer, Cambridge, MA (US); Stephan DeCarlo, Cambridge, MA (US); Praveen Tiwari, Boston, MA (US); Praveen Kokkonda, Boston, MA (US); Veronika Shoba, Cambridge, MA (US); Arghya Deb, Cambridge, MA (US); Sreekanth Vedagopuram, Boston, MA (US)

(73) Assignees: The Broad Institute, Inc., Cambridge, MA (US); President and Fellows of Harvard College, Cambridge, MA (US); The Brigham and Women's Hospital, Inc., Boston, MA (US)

(21) Appl. No.: 18/978,484

(22) Filed: Dec. 12, 2024

Related U.S. Application Data
(63) Continuation of application No. PCT/US2023/068349, filed on Jun. 13, 2023.
(60) Provisional application No. 63/351,736, filed on Jun. 13, 2022.

(51) Int. Cl. C07K 14/47; C12N 1/20; C12N 9/12; C12N 15/10; C12N 15/72

(52) U.S. Cl. CPC C07K 14/4702; C12N 1/20; C12N 9/1247; C12N 15/1058; C12N 15/72; C07K 2319/81; C12N 2795/14143; C12Y 207/07006

(57) ABSTRACT

Aspects of the disclosure relate to compositions and methods for targeted protein degradation. In some embodiments, the disclosure relates to methods of evolving protein degrons to interact with certain small molecule inducers (e.g., VS-777, PT-179, or PK-1016). In some embodiments, the disclosure relates to compositions (e.g., peptides, nucleic acids encoding the protein degrons, etc.) used for targeted protein degradation. In some embodiments, the disclosure relates to methods of degrading a target polypeptide in a cell.

### RELATED APPLICATIONS

This application claims priority under 35 U.S.C. § 119 (e) to U.S. Provisional Application, U.S. Ser. No. 63/351,736, filed Jun. 13, 2022, which is incorporated herein by reference.

### FEDERALLY SPONSORED RESEARCH

This invention was made with government support under R01 EB027793, R01 EB031172, R35 GM118062, and F32 GM133088 awarded by the National Institutes of Health. The government has certain rights in the invention.

### REFERENCE TO AN ELECTRONIC SEQUENCE LISTING

The contents of the electronic sequence listing (B119570164WO00-SEQ-CBD.xml; Size: 132,317 bytes; and Date of Creation: Jun. 9, 2023) is herein incorporated by reference in its entirety.

### BACKGROUND

Protein degradation plays a key role in nearly all cellular processes and is essential in maintaining cellular homeostasis. It has emerged as a powerful therapeutic modality over the past 10 years, especially since the discovery of small molecules that can direct the cellular machinery to selectively target proteins for degradation in the cell, rather than simply inhibiting protein activity. Small molecules known as molecular glues are able to stabilize the interaction between two proteins that do not normally interact, and cause degradation of the protein harnessing the cell's natural proteosomal pathway. These small molecule inducers of degradation, offer therapeutic accessibility to a broad family of target proteins previously thought to be undruggable, as they could not be modulated via ways of traditional pharmaceuticals.

### SUMMARY

Aspects of the disclosure relate to compositions and methods for targeted protein degradation. The disclosure is based, in part, on evolved protein degrons that interact with certain non-canonical cereblon (CRBN) substrates (e.g., small molecule substrates) to mediate degradation of proteins containing the degrons. In some embodiments, evolved protein degrons described by the disclosure have increased sensitivity (e.g., binding affinity and specificity) to small molecule-bound CRBN with certain small molecule substrates (e.g., modified thalidomide analogs, for example, PT-179) relative to previously described CRBN substrates, such as immunomodulatory imide drugs (IMiDs) including thalidomide and/or its analogs.

It has previously been observed that a ternary complex forms between cereblon (CRBN), pomalidomide, and a “super degron” (SEQ ID NO: 1), and certain small molecules (e.g., VS-777, PT-179, PK-1016, etc.) containing modifications that disrupt the interaction between the super degron and the cereblon-IMiD complex have been developed. As described further in the Examples section below, successive rounds of phage-assisted evolution (e.g., phage-assisted continuous evolution (PACE) and/or phage-assisted non-continuous evolution (PANCE) techniques) were performed using small molecules (e.g., VS-777, PT-179, PK-1016) to identify mutations in the super degron that rescued CRBN-IMiD ternary complex formation with high affinity. In some embodiments, protein degrons are evolved from a previously described “super degron”, SD0, comprising the amino acid sequence set forth in SEQ ID NO: 1. In some embodiments, evolved variants of the super degron are re-engineered to form a strong ternary complex with small molecule-bound CRBN. In some embodiments, the small molecule is VS-777, PT-179, or PK-1016. In some embodiments, the small molecule is PT-179. In some embodiments, evolved protein degrons have increased sensitivity (e.g., binding affinity and selectivity) to small molecule-bound CRBN, wherein the small molecule bound to CRBN is a small molecule other than thalidomide and/or its analogs. In some embodiments, evolved protein degrons provided herein serve as potent small molecule responsive degron tags for targeted protein degradation.

Accordingly, in some aspects, the disclosure provides a protein degron comprising an amino acid sequence that is at least 50% (e.g., at least 60%, 65%, 70%, 75%, 80%, 95%, 90%, 95%, or 99%) identical to the amino acid sequence set forth in SEQ ID NO: 1 and comprises one or more amino acid substitutions at one or more positions recited in Table 1 or Table 2.

In some embodiments, a protein degron comprises an amino acid sequence that is at least 60%, 65%, 70%, 75%, 80%, 95%, 90%, 95%, or 99% identical to the amino acid sequence set forth in SEQ ID NO: 1. In some embodiments, a protein degron is no more than 99.9% identical to the amino acid sequence set forth in SEQ ID NO: 1. In some embodiments, a protein degron is not naturally occurring.

In some embodiments, a protein degron comprises one or more amino acid substitutions at a position selected from F1, V3, M5, V6, H7, K8, S10, T12, E14, R15, P16, L17, Q18, E20, 121, T25, Q28, K29, G30, N31, K37, T40, G41, E42, P44, F45, K46, C47, C50, N51, A53, C54, R57, D58, A59, and L60 relative to SEQ ID NO: 1.

In some embodiments, a protein degron comprises 1, 2, 3, 4, 5, 6, 7, 8, 9, 10, 11, 12, 13, 14, or 15 amino acid substitutions relative to SEQ ID NO: 1.

In some embodiments, amino acid substitutions are selected from FIL, V3E, V3A, M5L, V6G, H7Y, K8E, K8R, S10R, T12P, E14D, R15L, P16S, P16L, L17F, Q18M, Q18I, Q18H, Q18F, E20K, E20P, E20R, I21V, T25M, Q28E, Q28K, K29E, G30V, N31K, N31D, N31T, K37N, T40M, T40P, G41D, E42V, P44L, P44T, P44M, F45V, F45L, K46R, K46stop, C47Y, C50Y, C50R, N51K, N51H, A53D, C54Y, R57K, D58R, D58N, A59C, and L60F relative to SEQ ID NO: 1.

In some embodiments, a protein degron comprises 1, 2, 3, 4, 5, 6, 7, 8, 9, 10, 11, 12, 13, 14, or 15 amino acid substitutions selected from FIL, V3E, V3A, M5L, V6G, H7Y, K8E, K8R, S10R, T12P, E14D, R15L, P16S, P16L, L17F, Q18M, Q18I, Q18H, Q18F, E20K, E20P, E20R, 121V, T25M, Q28E, Q28K, K29E, G30V, N31K, N31D, N31T, K37N, T40M, T40P, G41D, E42V, P44L, P44T, P44M, F45V, F45L, K46R, K46stop, C47Y, C50Y, C50R, N51K, N51H, A53D, C54Y, R57K, D58R, D58N, A59C, and L60F relative to SEQ ID NO: 1.

In some embodiments, a protein degron comprises 1, 2, 3, 4, 5, 6, 7, or 8 amino acid substitutions selected from R15L, P16L, Q18F, E20P, K37N, P44L, C47Y, and C50Y relative to SEQ ID NO: 1. In some embodiments, a protein degron comprises the following amino acid substitutions relative to SEQ ID NO: 1: R15L, P16L, Q18F, E20P, K37N, P44L, C47Y, and C50Y. In some embodiments, a protein degron comprises 1, 2, 3, 4, 5, 6, 7, 8, 9, 10, or 11 amino acid substitutions selected from R15L, P16L, Q18F, E20P, N31T, K37N, T40P, P44L, K46R, C47Y, and C50Y relative to SEQ ID NO: 1. In some embodiments, a protein degron comprises the following amino acid substitutions relative to SEQ ID NO: 1: R15L, P16L, Q18F, E20P, N31T, K37N, T40P, P44L, K46R, C47Y, and C50Y.

In some embodiments, a protein degron comprises an amino acid sequence that is at least 70% sequence identical to the amino acid sequence set forth in any one of SEQ ID NOs.: 2-45 or 54-58. In some embodiments, a protein degron comprises an amino acid sequence that is at least 75%, at least 80%, at least 85%, at least 90%, or at least 99% identical to the amino acid sequence set forth in any one of SEQ ID NOs.: 2-45, 54-58, 124, or 125. In some embodiments, a protein degron comprises or consists of the amino acid sequence set forth in any one of SEQ ID NOs: 2-45, 54-58, 124, or 125. In some embodiments, a protein degron is not naturally occurring.

In some embodiments, a protein degron comprises an amino acid sequence that is at least 70% sequence identical to the amino acid sequence set forth in SEQ ID NO: 37. In some embodiments, a protein degron comprises an amino acid sequence that is at least 75%, at least 80%, at least 85%, at least 90%, or at least 99% identical to the amino acid sequence set forth in SEQ ID NO: 37. In some embodiments, a protein degron comprises the amino acid sequence set forth in SEQ ID NO: 37. In some embodiments, a protein degron comprises an amino acid sequence that is at least 70% sequence identical to the amino acid sequence set forth in SEQ ID NO: 125. In some embodiments, a protein degron comprises an amino acid sequence that is at least 75%, at least 80%, at least 85%, at least 90%, or at least 99% identical to the amino acid sequence set forth in SEQ ID NO: 125. In some embodiments, a protein degron comprises or consists of the amino acid sequence set forth in SEQ ID NO: 125.

In some aspects, the disclosure provides truncated variants of protein degrons. In some aspects, the disclosure provides a truncated protein degron comprising an amino acid sequence that is at least 50% (e.g., at least 50%, 55%, 60%, 65%, 70%, 75%, 80%, 85%, 90%, 95%, or 99%) identical to amino acid residues 15-50 of SEQ ID NO: 1 and comprises one or more amino acid substitutions at one or more positions selected from R15, P16, Q18, E20, K37, P44, C47, and C50, relative to SEQ ID NO: 1. In some embodiments, the truncated protein degron lacks one or more amino acids at one or more of the following ranges of positions: 1-14, 1-15, 40-60, 45-60, 48-60, 51-60, and 53-60, relative to SEQ ID NO: 1.

In some embodiments, a truncated protein degron comprises one or more amino acid substitutions are selected from R15L, P16L, Q18F, E20P, K37N, P44L, C47Y, and C50Y relative to SEQ ID NO: 1. In some embodiments, a truncated protein degron comprises the following amino acid substitutions relative to SEQ ID NO: 1: R15L, P16L, Q18F, E20P, K37N, P44L, C47Y, and C50Y.

In some embodiments, a truncated protein degron comprises an amino acid sequence that is at least 70% (e.g., at least 70%, 75%, 80%, 85%, 90%, 95%, or 99%) identical to the amino acid sequence set forth in any one of SEQ ID NOs.: 46-53. In some embodiments, a truncated protein degron comprises an amino acid sequence that is at least 75%, at least 80%, at least 85%, at least 90%, or at least 99% identical to the amino acid sequence set forth in any one of SEQ ID NOs.: 46-53. In some embodiments, a truncated protein degron comprises or consists of the amino acid sequence set forth in any one of SEQ ID NOs: 46-53. In some embodiments, a truncated protein degron is not naturally occurring.

In some embodiments, a truncated protein degron comprises an amino acid sequence that is at least 70% identical to the amino acid sequence set forth in SEQ ID NO: 49. In some embodiments, a truncated protein degron comprises an amino acid sequence that is at least 75%, at least 80%, at least 85%, at least 90%, or at least 99% identical to the amino acid sequence set forth in SEQ ID NO: 49. In some embodiments, a truncated protein degron comprises or consists of the amino acid sequence set forth SEQ ID NO: 49.

In some embodiments, a protein degron binds to cereblon (CRBN) protein in the presence of a small molecule CRBN substrate. In some embodiments, the small molecule CRBN substrate is or comprises VS-777, PT-179, or PK-1016. In some embodiments, a small molecule CRBN substrate is PT-179, and is of the structure set forth below:

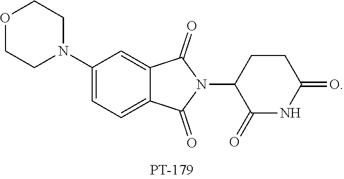 [SMILES: O=C1CCC(N2C(=O)c3ccc(N4CCOCC4)cc3C2=O)C(=O)N1]

In some aspects, the disclosure provides a nucleic acid sequence that encodes a protein degron as described herein. In some embodiments, the nucleic acid sequence is the nucleic acid sequence set forth in any one of SEQ ID NOs.: 59-95, and 128-129.

In some embodiments, a nucleic acid sequence comprises at least 70% identity to the nucleic acid sequence set forth in any one of SEQ ID NOs.: 59-95. In some embodiments, a nucleic acid sequence comprises at least 70% (e.g., at least 70%, 75%, 80%, 85%, 90%, 95%, or 99%) identity to the nucleic acid sequence set forth in any one of SEQ ID NOs.: 128-129. In some embodiments, a nucleic acid sequence comprises at least 70% (e.g., at least 70%, 75%, 80%, 85%, 90%, 95%, or 99%) identity to the nucleic acid sequence set forth in any one of SEQ ID NOs.: 96-123, 126, and 127. In some embodiments, a nucleic acid sequence comprises at least 75%, at least 80%, at least 85%, at least 90%, at least 95%, at least 96%, at least 97%, at least 98%, or at least 99% identity to the nucleic acid sequence set forth in any one of SEQ ID NOs: 59-95. In some embodiments, a nucleic acid sequence comprises at least 75%, at least 80%, at least 85%, at least 90%, at least 95%, at least 96%, at least 97%, at least 98%, or at least 99% identity to the nucleic acid sequence set forth in any one of SEQ ID NOs: 128-129. In some embodiments, a nucleic acid sequence comprises at least 75%, at least 80%, at least 85%, at least 90%, at least 95%, at least 96%, at least 97%, at least 98%, or at least 99% identity to the nucleic acid sequence set forth in any one of SEQ ID NOs: 96-123, 126, and 127. In some embodiments, a nucleic acid sequence comprises the sequence set forth in any one of SEQ ID NOs: 59-95, and 128-129.

In some aspects, the disclosure provides a vector comprising a nucleic acid encoding a protein degron as described herein. In some embodiments, the expression vector is a phage, plasmid, cosmid, bacmid, or viral vector. In some embodiments, the vector comprises a nucleic acid comprising the sequence set forth in any one of SEQ ID NOs: 59-95. In some embodiments, the vector comprises a nucleic acid comprising the sequence set forth in any one of SEQ ID NOs: 128-129. In some embodiments, the vector comprises a nucleic acid comprising the sequence set forth in any one of SEQ ID NOs: 96-123, 126, and 127.

In some aspects, the disclosure provides a host cell comprising a protein degron, a nucleic acid, or vector as described herein. In some embodiments, the host cell is a bacterial cell. In some embodiments, the host cell is an E. coli cell. In some embodiments, the host cell is a mammalian cell. In some embodiments, the host cell is a human cell.

In some aspects, the disclosure provides a complex comprising a cereblon (CRBN) protein simultaneously bound to a small molecule CRBN substrate and a protein degron as described herein. In some embodiments, the small molecule CRBN substrate is VS-777, PT-179, or PK-1016. In some embodiments, the small molecule CRBN substrate is PT-179 of the structure set forth below:

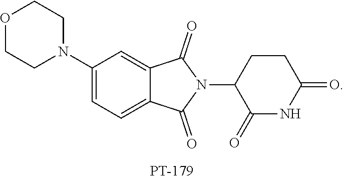 [SMILES: O=C1CCC(N2C(=O)c3ccc(N4CCOCC4)cc3C2=O)C(=O)N1]

In some embodiments, the complex further comprises one or more E3 ubiquitin ligase complex proteins. In some embodiments, one or more E3 ubiquitin ligase complex proteins are selected from damaged DNA binding protein 1 (DDB1), Cullin-4A (CUL4A), and regulator of cullins 1 (ROC1). In some embodiments, the complex further comprises at least one ubiquitin.

In some embodiments, a protein degron of a complex is associated with a protein. In some embodiments, a protein degron of a complex is connected to a protein. In some embodiments, the protein is a recombinant protein. In some embodiments, the protein is a fusion protein comprising the protein degron and a protein. In some embodiments, the protein is a fusion protein comprising the protein degron and a therapeutic protein. Examples of therapeutic proteins include, but are not limited to, antibodies, antibody fragments (e.g., single chain antibodies, etc.), therapeutic peptides (e.g., gene replacement therapies), toxins, chimeric antigen receptor (CAR) components, etc.

In some aspects, the disclosure provides a method of degrading a target protein in a cell, wherein the method comprises contacting a cell comprising cereblon (CRBN), and a target protein having a protein degron as described herein, with a small molecule CRBN substrate.

In some embodiments, a target protein is an endogenous protein (e.g., a protein endogenous to the cell). In some embodiments, a target protein is a recombinant protein (e.g., a protein that is heterologous with respect to the cell). In some embodiments, the target polypeptide is a therapeutic protein.

In some embodiments, the cell is in a subject. In some embodiments, the subject is a mammalian subject. In some embodiments, the subject is a human.

In some embodiments, the small molecule CRBN substrate is not thalidomide, lenalidomide, pomalidomide, avadomide, or iberdomide. In some embodiments, the small molecule CRBN substrate comprises VS-777, PT-179, or PK-1016. In some embodiments, a small molecule CRBN substrate is PT-179, and comprises the structure set forth below:

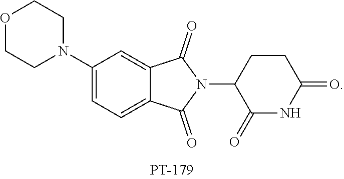 [SMILES: O=C1CCC(N2C(=O)c3ccc(N4CCOCC4)cc3C2=O)C(=O)N1]

In some aspects, the disclosure provides a method for evolving a protein degron. In some embodiments, the method comprises contacting a population of bacterial host cells with a population of phages comprising a first nucleic acid encoding a first fusion protein, and deficient in a full-length pIII gene. In some embodiments, the first fusion protein comprises a protein degron of interest and an RNA polymerase subunit. In some embodiments, the population of phages allows for expression of the first fusion protein in the host cells, and the host cells are suitable for phage infection, replication, and packaging. In some embodiments, the host cells comprise a second nucleic acid encoding full-length pIII protein, and a third nucleic acid sequence encoding a second fusion protein. In some embodiments, the second fusion protein comprises a cereblon (CRBN) and a repressor element, wherein expression of the pIII gene is dependent on interaction of the protein degron of interest of the first fusion protein with the CRBN of the second fusion protein. In some embodiments, phages are filamentous phages. In some embodiments, phages are M13 phages. In some embodiments, the method further comprises incubating the population of host cells and M13 phages under conditions allowing for the modification of the third nucleic acid, the production of infectious M13 phage, and the infection of host cells with M13 phage. In some embodiments, the conditions allowing for the modification of the third nucleic acid, are the presence of a small molecule. In some embodiments, infected cells are removed from the population of host cells, and the population of host cells is replenished with fresh host cells that are not infected by M13 phage. In some embodiments, the method further comprises isolating a modified M13 phage replication product encoding an evolved variant of the first fusion protein from the population of host cells. In some embodiments, the method further comprises use of a mutagenesis plasmid. In some embodiments, an RNA polymerase subunit is RNA polymerase omega (RpoZ) subunit. In some embodiments, bacterial host cells are E. coli cells.

In some embodiments, the method of evolving comprises incubating the population of host cells and M13 phages with a small molecule CRBN substrate. In some embodiments, a small molecule CRBN substrate is PT-179, and is of the structure set forth below:

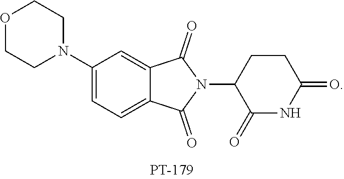 [SMILES: O=C1CCC(N2C(=O)c3ccc(N4CCOCC4)cc3C2=O)C(=O)N1]

In PACE, the gene for a protein of interest (POI) is placed on a selection phage (SP) in place of the phage gene gIII, which encodes the phage coat protein pIII. In some embodiments the POI is a protein degron. In some embodiments, a protein degron of interest comprises the amino acid sequence set forth in SEQ ID NO: 1. In some embodiments, host cells further comprise a helper plasmid and/or a mutagenesis plasmid. In some embodiments, a second nucleic acid encoding full-length pIII protein further comprises a promoter. In some embodiments, a promoter is a lacZ promoter or a mutant lacZ promoter. In some embodiments, a second nucleic acid encoding full-length pIII protein further comprises a repressor binding site. In some embodiments, a repressor binding site comprises an RR69 repressor binding site. In some embodiments, a repressor binding site comprises a sc-p22cI repressor binding site.

In some aspects, the disclosure provides a vector system comprising a first nucleic acid encoding a fusion protein comprising a protein degron of interest and an RNA polymerase subunit; a second nucleic acid encoding a full-length pIII protein; and a third nucleic acid encoding a fusion protein comprising cereblon (CRBN) and a phage repressor, wherein the nucleic acid sequence encoding the full-length pIII protein is under the control of a conditional promoter and comprises one or more phage repressor binding sites. In some embodiments, the protein degron of interest comprises the amino acid sequence set forth in SEQ ID NO: 1. In some embodiments, the phage repressor comprises a single-chain phage repressor. In some embodiments, the phage repressor comprises an RR69 phage repressor. In some embodiments, the phage repressor comprises a p22 phage repressor (e.g., sc-p22cI).

In some embodiments, the conditional promoter is a pLac-derived promoter. In some embodiments, the conditional promoter comprises a LacZ promoter or a mutant lacZ promoter.

In some embodiments, each nucleic acid of a vector system is on a separate vector. In some embodiments, each separate vector is independently selected from a phage vector or plasmid.

In some embodiments, a vector system further comprises a mutagenesis plasmid. In some embodiments, a mutagenesis plasmid comprises an arabinose-inducible promoter.

In some aspects, the disclosure provides a fusion protein comprising the protein degron as described herein and a target protein. In some embodiments, the target protein is an endogenous protein. In some embodiments, the target protein is a recombinant protein. In some embodiments, the target protein is a therapeutic protein. In some embodiments, the therapeutic protein is selected from the group consisting of antibodies, antibody fragments (e.g., single chain antibodies, etc.), therapeutic peptides (e.g., gene replacement therapies), toxins, or chimeric antigen receptor (CAR) components.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

FIG. 1 show representative data indicating that off-target neosubstrates of pomalidomide are not degraded by small molecules PT-179 an PK-1016, which feature a morpholine substitution at the 5-position of the phthalimide.

FIG. 2 shows representative data for unbiased off-target profiling of pomalidomide, PT-179, and PK-1016, which were utilized for the development of evolved degron proteins. PT-179 and PK-1016 showed a marked reduction in the number of differentially-expressed genes (DEGs) compared to pomalidomide.

FIG. 3 shows a schematic of one embodiment of phage-assisted evolution procedures (e.g., PACE and PANCE) for producing protein degrons.

FIG. 4 shows representative data for evolutionary variants observed during PANCE and PACE.

FIG. 5 shows an alignment of the starting super degron sequence, SD0 (SEQ ID NO: 1), an evolved degron sequence, SD36 (SEQ ID NO. 37), and a truncated (e.g., minimal) evolved degron sequence, SD40 (SEQ ID NO: 49). Structures of the small molecules, pomalidomide, PT-179, and PK-1016, used during the evolution of the degrons are also shown.

FIG. 6A shows representative data for ternary complex formation as measured through a PACE circuit transcriptional activation assay. FIG. 6B shows representative data for protein degradation of eGFP assessed by a flow-based degradation assay. The indicated degrons shown in the key are fused to the N-terminus of eGFP allowing measurement of eGFP:mCherry ratios for measuring degradation. FIG. 6C shows a Western Blot visualizing an SD40-eGFP construct (top) and a loading control, H2B (bottom), following overnight treatment with a range of PT-179 concentrations.

FIG. 7 shows a representative molecular model of the tertiary structure of a CRBN-IKZF1 (ZF2)-pomalidomide (6H0F) complex, a molecular model of the starting degron tag, SD0, and a model of an evolved degron tag, SD36 (SEQ ID NOs: 1, 37 from top to bottom).

FIGS. 8A-8C show a phage-assisted continuous evolution circuit for molecular glue complexes (MG-PACE). FIG. 8A shows a schematic of one embodiment of MG-PACE. In PACE, the gene for a protein of interest (POI) is placed on a selection phage (SP) in place of the phage gene gIII, which encodes the phage coat protein pIII. Host cells also harbor a mutagenesis plasmid (MP) that increases the incidence of mutation during SP replication an accessory plasmid (AP) encoding a selection circuit that provides pIII. FIG. 8B shows a Rapamycin-sensitive MG-PACE circuit. FIG. 8C shows a rapamycin-induced activation of the MG-PACE circuit.

FIGS. 9A-9I show phage assisted continuous evolution of new zinc finger (ZF) degrons. FIG. 9A shows structures of Pomalidomide and bumped IMiD analogs PT-179 and PK-1016. FIG. 9B shows a full-length CRBN MG-PACE circuit. FIG. 9C shows a CRBN-CTD MG-PACE circuit. FIG. 9D shows pomalidomide-induced activation for both MG-PACE CRBN circuits. FIG. 9E shows PACE in CRBN-CTD circuit. FIG. 9F shows PT-179-induced CRBN-CTD circuit activation with evolved variants. FIG. 9G shows PANCE and PACE in full-length CRBN circuit. FIG. 9H shows PT-179-induced full-length CRBN circuit activation with evolved variants. FIG. 9I shows amino acid substitutions in evolved protein degrons SD12, SD17, SD20, SD8, SD31, SD35, and SD36 relative to super degron, SD0. Sequences from top to bottom corresponding to SEQ ID NOs: 1, 13, 18, 21, 9, 32, 36, and 37.

FIG. 10A-10B show binding parameters of SD40. FIG. 10A shows PT-179-induced degradation of degron-fused GFP. FIG. 10B shows thermodynamic and kinetic binding parameters of initial and evolved ternary complexes.

FIGS. 11A-11D show representative data indicating that SD40 degrades ectopically- and endogenously-expressed tagged proteins. FIG. 11A shows the degradation of ectopically-expressed proteins fused to SD40. FIG. 11B shows rapid degradation of SD40-PKRKA. FIG. 11C shows editing efficiency BRD4 and PLK1. FIG. 11D shows degradation of endogenous tagged proteins.

FIG. 12A shows a schematic of one embodiment of the evolution of degrons that engage molecule-bound mouse CRBN. FIG. 12B shows representative plaque forming unit (pfu) data from 205 hours of PACE seeded with SD36-encoding phage. FIG. 12C shows representative data for PACE circuit activation as measured through a transcriptional activation assay. FIG. 12D shows representative data for protein degradation of eGFP assessed by a flow-based degradation assay. The indicated degron variants shown in the key are fused to eGFP allowing measurement of eGFP:mCherry ratios for measuring degradation. FIG. 12E shows amino acid substitutions in evolved protein degrons SD36, SD55, and SD56 relative to super degron, SD0 (SEQ ID NOs: 130-133 from top to bottom).

FIGS. 13A-13C show PT-179 degrades far fewer off-target neosubstrates than canonical IMiD pomalidomide.

FIGS. 14A-14B show representative data for binding affinity assessed by a fluorescence polarization assay. The affinity of CRBN to PT-179, pomalidomide, and PK-1016 was measured by competitive fluorescence anisostropy. FIG. 14C shows representative data for competitive CRBN engagement assessed an assay using bioluminescence resonance energy transfer (BRET) from an ectopically-expressed CRBN-NanoLuc fusion to a fluorescent IMiD conjugate in human cells.

FIGS. 15A-15B show SD0 secondary structure and CRBN·pomalidomide·IKZF1 co-crystal structure (SEQ ID NOs: 134 and 1 from top to bottom).

FIG. 16 shows representative plaque forming unit (pfu) data from 96 simultaneous PANCE experiments with IMiD analog evolutionary stepping stones.

FIGS. 17A-17D shows representative final plaque forming unit (pfu) data following IMiD analog stepping stone evolution.

FIGS. 18A-18B shows representative data following a negative selection PACE circuit activation.

FIG. 19A shows a schematic illustrating HEK293T cell lines expressing degron-tagged eGFP. FIG. 19B shows representative data for protein degradation of eGFP assessed by a flow-based degradation assay. The indicated degron variants shown in the key are fused to eGFP allowing measurement of eGFP:mCherry ratios for measuring degradation. FIG. 19C shows representative data for ternary complex formation as measured through a PACE circuit transcriptional activation assay.

FIG. 20A shows trimming of degron variants SD40, SD41, and SD42 beyond residues 15 through 50 (SEQ ID NOs: 49-51 from top to bottom). FIG. 20B shows PT-179-induced degradation of degron-fused GFP. FIG. 20C shows pomalidomide-induced degradation of degron-fused GFP.

FIGS. 21A-21D relate to bio-layer interferometry (BLI), which was conducted to compare the affinity of the evolved ternary complex CRBN·PT-179·SD40 to that of the original complex CRBN·pomalidomide·SD0. FIG. 21A illustrates the sequences of SD0 and SD40. FIG. 21B shows a schematic illustrating BLI (SEQ ID NOs: 135 and 49 from top to bottom). FIGS. 21C-21D show representative data for association and dissociation rates of DDB1·CRBN precomplexed with either PT-179 or pomalidomide as measured by BLI with immobilized maltose binding protein (MBP)-degron.

### DEFINITIONS

The term “protein,” as used herein, refers to a polymer of amino acid residues linked together by peptide bonds. The term, as used herein, refers to proteins, polypeptides, and peptides of any size, structure, or function. Typically, a protein will be at least three amino acids long but is generally longer than 50 amino acids in length. A protein may refer to an individual protein or a collection of proteins. Inventive proteins preferably contain only natural amino acids, although non-natural amino acids (i.e., compounds that do not occur in nature but that can be incorporated into a polypeptide chain; see, for example, cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive protein may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein may also be a single molecule or may be a multi-molecular complex. A protein may be just a fragment of a naturally occurring protein or peptide. A protein may be naturally occurring, recombinant, or synthetic, or any combination of these. In some embodiments, the protein is a degron.

The term “peptide”, as used herein, refers to a short, contiguous chain of amino acids linked to one another by peptide bonds. Generally, a peptide ranges from about 2 amino acids to about 50 amino acids in length (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length) but may be longer in the case of a polypeptide. In some embodiments, a peptide is a fragment or portion of a larger protein, for example comprising one or more domains of a larger protein. Peptides may be linear (e.g., branched, unbranched, etc.) or cyclic (e.g., form one or more closed rings). A “polypeptide”, as used herein, refers to a longer (e.g., between about 50 and about 100), continuous, unbranched peptide chain.

The term “protein degron”, “degron”, or “degron tag” as used herein, refers to an amino acid sequence that when added to or part of a target protein causes that protein to be degraded upon addition of a small molecule. In some embodiments, a degron tag binds to cereblon (CRBN) in the presence of one or more small molecule CRBN substrates, for example, IMiDs including thalidomide, analogues of thalidomide, or other modified thalidomide derivatives, and mediates ubiquitination of a protein containing the protein degron by CRBN. Degrons are protein sequences that are important in regulating the rate of a protein's degradation, and form the basis for many molecular glue degraders. The general concept of molecular glue degraders has been described, for example, in Dong et al., J Med Chem. 64 (15): 10606-10620, 2021.

Degrons may include short amino acid sequences, structural motifs, and exposed amino acids (e.g., lysine or arginine). In some embodiments, the degron tag is necessary for recruiting a target protein's cognate ubiquitin ligase complex, which, in turn, marks the protein for degradation by proteolysis. In some embodiments, the degron tag is ubiquitin-dependent. In some embodiments, the degron tag is ubiquitin-independent. In some embodiments, degrons provided herein are evolved from a starting degron (SD0), referred to as a “super degron” (SEQ ID NO: 1). In some embodiments, the degron comprises a zinc finger polypeptide. In some embodiments, the zinc finger comprises a Cys2 His2 (C2H2) domain. C2H2 domains are well known, for example, as described by Brayer et al., Cell Biochem Biophys. 2008; 50 (3): 111-31 and Sievers et al., Science. 362 (6414): eaat0572, 2018.

The term “super degron” as used herein, refers to a C2H2 zinc finger-derived protein capable of mediating drug-dependent degradation (e.g., IMiD-induced degradation) more efficiently than any present in the human proteome, for example, as described in published international application, WO 2021/188286 A2, the entire contents of which are incorporated by reference herein. In some embodiments, a super degron is a protein comprising the 60 amino acid sequence: FNVLMVHKRSHTGERPLQCEICGFTCRQKGNLLRHIKLHTGEKPFKCHLCNYACQRR DAL (SEQ ID NO: 1). In some embodiments, the super degron forms a ternary complex with cereblon and certain IMiDs, for example, pomalidomide.

The term “cereblon” or “CRBN,” as used herein, refers to a 442-amino acid protein that is the substrate receptor of the CRL4^{CRBN} E3 ubiquitin ligase complex, and is involved in mediating the ubiquitination and subsequent proteasomal degradation of target proteins. In some embodiments, cereblon comprises the amino acid sequence set forth in NCBI Reference Sequence Accession Number NP_057386.2 or NP_001166953.

E3 ubiquitin ligase complexes select proteins for degradation through the recognition of degrons, specific amino acid that are sufficient to promote ubiquitylation and degradation when embedded in a substrate. Cereblon is a molecular binding target of small molecules including immunomodulatory drugs (IMiDs), such as thalidomide and its analogs. Upon binding of IMiDs to CRBN, the substrate specificity of CRBN is altered to target specific protein targets for destruction, sometimes referred to as “neosubstrates”, including, but not limited to, transcription factors IKZF1 (Ikaros) and IKZF3 (Aiolos), as well as CK1α (Casein kinase 1 alpha), GSPT1, SALL4, p63, GZF1, ZFP91, ZNF98, ZNF276, ZNF653, ZNF692, and ZNF827. In some embodiments, cereblon is a molecular binding target of small molecules VS-777, PT-179, or PK-1016. In some embodiments, cereblon is a molecular binding target of PT-179.

The term “cereblon substrate” or “CRBN substrate,” as used herein, refers to a molecule which binds to cereblon and induces a structural change in cereblon that results in cereblon being able to bind one or more neosubstrates, for example, one or more degron tags. In some embodiments, a CRBN substrate is a small molecule, for example, an immunomodulatory imide drug (IMiD), or a modified IMiD, such as VS-777, PT-179, or PK-1016.

The term “E3 ubiquitin ligase” or “E3 ligase” refers to any protein that recruits an E2 ubiquitin-conjugating enzyme that has been loaded with ubiquitin, recognizes a protein substrate, and assists or directly catalyzes the transfer of ubiquitin from the E2 protein to the protein substrate. The transfer of the ubiquitin tag to the protein substrate targets it for destruction by the proteasome. Members of the E3 ubiquitin ligase complex include, but are not limited to, damaged DNA binding protein 1 (DDB1), Cullin-4A (CUL4A), and regulator of cullins 1 (ROC1).

The term “immunomodulatory imide drug” or “IMiD” as used herein, refers to an immunomodulatory drug containing an imide group, including, but not limited to, thalidomide, or an analog thereof (e.g., pomalidomide, lenalidomide, avadomide, and iberdomide). Generally, IMiDs share a common glutarimide moiety connected to a second moiety typically derived from phtaloyl. IMiDs have shown significant efficacy in the treatment of multiple myeloma (MM), myelodysplastic syndrome (MDS) with deletion of chromosome 5q (del (5q)) and other hematological malignancies. In some embodiments, IMiDs target the protein cereblon. Via the glutarimide moiety, IMiDs are able to bind to a tri-tryptophan pocket within the thalidomide-binding domain of cereblon. In some embodiments, IMiDs are referred to as cereblon modulators. Non-limiting examples of IMiDs are shown below:

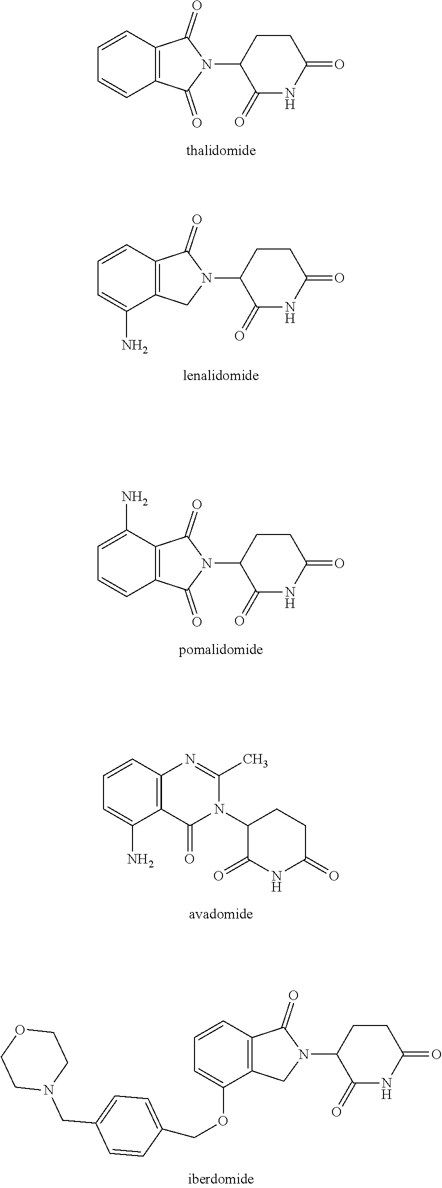 [SMILES: O=C1CCC(N2C(=O)c3ccccc3C2=O)C(=O)N1 ; Nc1cccc2c1CN(C1CCC(=O)NC1=O)C2=O ; Nc1cccc2c1C(=O)N(C1CCC(=O)NC1=O)C2=O ; Cc1nc2cccc(N)c2c(=O)n1C1CCC(=O)NC1=O ; O=C1CCC(N2Cc3c(OCc4ccc(CN5CCOCC5)cc4)cccc3C2=O)C(=O)N1]

In some embodiments, the IMiD or analog thereof is modified. In some embodiments, the modified IMiD is a derivative of pomalidomide. In some embodiments, the modified IMiD is modified at the 5-position of the phthalimide relative to pomalidomide. In some embodiments, the modified IMiD contains morpholine substitutions at the 5-position of the phthalimide relative to pomalidomide. Non-limiting examples of modified IMiDs include VS-777, PT-179, and PK-1016, structures of which are shown below:

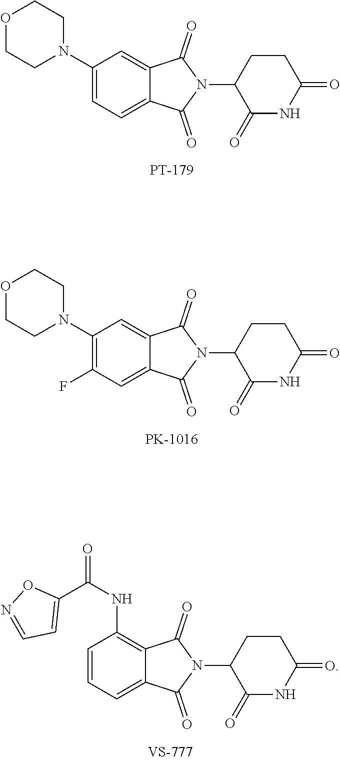 [SMILES: O=C1CCC(N2C(=O)c3ccc(N4CCOCC4)cc3C2=O)C(=O)N1 ; O=C1CCC(N2C(=O)c3cc(F)c(N4CCOCC4)cc3C2=O)C(=O)N1 ; O=C1CCC(N2C(=O)c3cccc(NC(=O)c4ccno4)c3C2=O)C(=O)N1]

The term “analog” refers to a molecule that is not identical, but has similar functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's membrane permeability or half-life, without altering, for example, ligand binding. An analog may include an unnatural amino acid. Thalidomide analogs include, but are not limited to, pomalidomide, lenalidomide, avadomide, or iberdomide.

The term “small molecule” refers to molecules, whether naturally-occurring or artificially created (e.g., via chemical synthesis) that have a relatively low molecular weight. In some embodiments, the small molecule is found in the body. Typically, a small molecule is an organic compound (e.g., it contains carbon). A small molecule may be an inorganic compound in some embodiments. The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, at least about 1,000 g/mol, at least about 1,100 g/mol, at least about 1,200 g/mol, at least about 1,300 g/mol, at least about 1,400 g/mol, at least about 1,500 g/mol, at least about 2,000 g/mol, at least about 2,500 g/mol, or at least about 3,000 g/mol. Combinations of the above ranges (e.g., at least about 200 g/mol and not more than about 500 g/mol) are also possible.

In some embodiments, a small molecule is a modulator of an E3 ligase that scaffolds protein-protein interactions. In some embodiments, the modulator is a cereblon modulator that scaffolds direct protein-protein interactions between the CRL4^{CRBN} E3 ubiquitin ligase and substrate, exemplifying the molecular glue mechanism. In some embodiments, the cereblon modulator is thalidomide, lenalidomide, pomalidomide, avadomide, or iberdomide. In some embodiments, the cereblon modulator is not thalidomide, lenalidomide, pomalidomide, avadomide, or iberdomide. In some embodiments, the cereblon modulator is VS-777, PT-179, or PK-1016. VS-777, PT-179, and PK-1016 contain substitutions that disrupt the interaction between the super degron and the cereblon-IMiD complex. PT-179, and PK-1016 contain morpholine substitutions at the 5-position of the phthalimide relative to pomalidomide. VS-777 contains a substitution at the 4-position of the phthalimide relative to pomalidomide. In some embodiments, the cereblon modulator is PT-179. In some embodiments, provided herein are evolved protein degrons with increased sensitivity to small molecule-bound CRBN, wherein the small molecules bound to CRBN is a small molecule other than thalidomide and/or its analogues. In some embodiments, provided herein are evolved protein degrons with increased sensitivity (e.g., binding affinity and selectivity) to VS-777-bound CRBN, PT-179-bound CRBN, or PK-1016-bound CRBN. In some embodiments, provided herein are evolved protein degrons with increased sensitivity (e.g., binding affinity and selectivity) to PT-179-bound CRBN.

The term “cereblon modulator” or “CRBN modulator” refers to any agent which binds cereblon (CRBN) and alters an activity of CRBN. In some embodiments, an activity of CRBN includes binding with and/or mediating degradation of transcription factors and/or kinases including but not limited to IKZF1, IKZF3, or CK1a. In some embodiments, a cereblon modulator includes agents that alter binding of CRBN with transcription factors and/or kinases, including, but not limited to, IKZF1, IKZF3, and CK1α and agents that alter CRBN's mediation of transcription factors including but not limited to IKZF1, IKZF3, or CK1α degradation. In some embodiments, a modulator of CRBN is a small molecule.

The term “molecular glue,” as used herein, refers to a small molecule that stabilizes the interaction between two proteins that do not normally interact. Examples of molecular glues that induce degradation of protein targets include IMiDs, which generate a protein-protein interaction between a substrate and cereblon. In some embodiments, a small molecule modulator is a molecular glue degrader. Molecular glue degraders are a class of small molecules that have been shown to induce degradation of target proteins commonly considered ‘undruggable’, as they could not be blocked via ways of traditional pharmacology.

The term “protein degron variant” or “evolved protein degron” as used herein, refers to a protein having one or more amino acid variations introduced into the amino acid sequence, e.g., as a result of application of PACE/PANCE or by genetic engineering (e.g., recombinant gene expression, gene synthesis, etc.), as compared to the amino acid sequence of a super degron (SEQ ID NO: 1). Amino acid sequence variations may include one or more mutated residues within the amino acid sequence of the protein degron, e.g., as a result of a substitution of one amino acid for another, deletions of one or more amino acids (e.g., a truncated protein), insertions of one or more amino acids, or any combination of the foregoing. In certain embodiments, the evolved protein degron comprises the amino acid sequence set forth in any one of SEQ ID NOs: 2-45 or 54-58. In some embodiments, the evolved protein degron comprises or consists of the amino acid sequence set forth in any one of SEQ ID NOs: 124 or 125. In some embodiments, the evolved protein degron comprises the amino acid sequence set forth in SEQ ID NO: 37. In some embodiments, the evolved protein degron consists of the amino acid sequence set forth in SEQ ID NO: 37. In some embodiments, the evolved protein degron comprises or consists of the amino acid sequence set forth in SEQ ID NO: 125.

The term “continuous evolution,” as used herein, refers to an evolution process, in which a population of nucleic acids encoding a protein of interest (e.g. protein degron) is subjected to multiple rounds of: (a) replication, (b) mutation (or modification of the nucleic acids in the population), and (c) selection to produce a desired evolved product, for example, a novel nucleic acid encoding a novel protein with a desired activity (e.g., ability to bind with small molecule-bound CRBN), wherein the multiple rounds of replication, mutation, and selection can be performed without investigator interaction, and wherein the processes (a)-(c) can be carried out simultaneously. Typically, the evolution procedure is carried out in vitro, for example, using cells in culture as host cells (e.g., bacterial cells). During a continuous evolution process, the population of nucleic acids replicates in a flow of host cells, e.g., a flow through a lagoon. In general, a continuous evolution process provided herein relies on a system in which a gene of interest is provided in a nucleic acid vector that undergoes a life-cycle including replication in a host cell and transfer to another host cell, wherein a critical component of the life-cycle is deactivated, and reactivation of the component is dependent upon a desired variation in an amino acid sequence of a protein encoded by the gene of interest.

In some embodiments, the gene of interest (e.g., a gene encoding a protein degron) is transferred from cell to cell in a manner dependent on the activity of the gene of interest. In some embodiments, the transfer vector is a virus infecting cells, for example, a bacteriophage or a retroviral vector. In some embodiments, the viral vector is a phage vector that infects bacterial host cells. In some embodiments, the transfer vector is a conjugative plasmid transferred from a donor bacterial cell to a recipient bacterial cell.

In some embodiments, the nucleic acid vector comprising the gene of interest (e.g., a gene encoding a protein degron) is a phage, a viral vector, or naked DNA (e.g., a mobilization plasmid). In some embodiments, transfer of the gene of interest from cell to cell is via infection, transfection, transduction, conjugation, or uptake of naked DNA, and efficiency of cell-to-cell transfer (e.g., transfer rate) is dependent on an activity of a product encoded by the gene of interest. For example, in some embodiments, the nucleic acid vector is a phage harboring the gene of interest and the efficiency of phage transfer (via infection) is dependent on an activity of the gene of interest in that a protein required for the generation of phage particles (e.g., pIII for M13 phage) is expressed in the host cells only in the presence of the desired activity of the gene of interest (e.g., ability to bind with small molecule-bound CRBN).

Some embodiments provide a continuous evolution system, in which a population of viral vectors comprising a gene of interest to be evolved replicates in a flow of host cells, e.g., a flow through a lagoon (e.g., evolution vessel), wherein the viral vectors are deficient in a gene (e.g. full-length pIII gene) encoding a protein that is essential for the generation of infectious viral particles, and wherein that gene is in the host cell under the control of a conditional promoter that can be activated by a gene product encoded by the gene of interest (e.g. gene encoding a protein degron of interest), or a mutated version thereof. In some embodiments, the activity of the conditional promoter depends on a desired function of a gene product encoded by the gene of interest (e.g. gene encoding a protein degron of interest). Viral vectors, in which the gene of interest (e.g. gene encoding a protein degron of interest) has not acquired a desired function as a result of a variation of amino acids introduced into the gene product protein sequence, will not activate the conditional promoter, or may only achieve minimal activation, while any mutations introduced into the gene of interest that confers the desired function will result in activation of the conditional promoter. Since the conditional promoter controls an essential protein for the viral life cycle, e.g., pIII, activation of this promoter directly corresponds to an advantage in viral spread and replication for those vectors that have acquired an advantageous mutation.

The term “flow,” as used herein in the context of host cells, refers to a stream of host cells, wherein fresh host cells are being introduced into a host cell population, for example, a host cell population in a lagoon, remain within the population for a limited time, and are then removed from the host cell population. In a simple form, a host cell flow may be a flow through a tube, or a channel, for example, at a controlled rate. In other embodiments, a flow of host cells is directed through a lagoon that holds a volume of cell culture media and comprises an inflow and an outflow. The introduction of fresh host cells may be continuous or intermittent and removal may be passive, e.g., by overflow, or active, e.g., by active siphoning or pumping. Removal further may be random, for example, if a stirred suspension culture of host cells is provided, removed liquid culture media will contain freshly introduced host cells as well as cells that have been a member of the host cell population within the lagoon for some time. Even though, in theory, a cell could escape removal from the lagoon indefinitely, the average host cell will remain only for a limited period of time within the lagoon, which is determined mainly by the flow rate of the culture media (and suspended cells) through the lagoon.

Since the viral vectors replicate in a flow of host cells, in which fresh, uninfected host cells are provided while infected cells are removed, multiple consecutive viral life cycles can occur without investigator interaction, which allows for the accumulation of multiple advantageous mutations in a single evolution experiment.

The term “phage-assisted continuous evolution” (also used interchangeably herein with “PACE”), as used herein, refers to continuous evolution that employs phage as viral vectors. The general concept of PACE technology has been described, for example, in U.S. Pat. No. 9,023,594, issued May 5, 2015; U.S. Pat. No. 9,771,574, issued Sep. 26, 2017; U.S. patent application Ser. No. 15/713,403, filed Sep. 22, 2017 (now abandoned); International PCT Application PCT/US2009/056194, filed Sep. 8, 2009, published as WO 2010/028347 on Mar. 11, 2010; U.S. Provisional Patent Application Ser. No. 61/426,139, filed Dec. 22, 2010; U.S. Pat. No. 9,394,537, issued Jul. 19, 2016; U.S. Pat. No. 10,336,997, issued Jul. 2, 2019; U.S. Pat. No. 11,214,792, issued Jan. 4, 2022; International PCT Application PCT/US2011/066747, filed Dec. 22, 2011, published as WO 2012/088381 on Jun. 28, 2012; U.S. Provisional Patent Application Ser. No. 61/929,378 filed Jan. 20, 2014; U.S. Pat. No. 10,179,911, issued Jan. 15, 2019; U.S. patent application Ser. No. 16/238,386, filed Jan. 2, 2019; International PCT Application PCT/US2015/012022, filed Jan. 20, 2015; U.S. Provisional Patent Application Ser. No. 62/158,982, filed May 8, 2015; U.S. Provisional Patent Application Ser. No. 62/187,669, filed Jul. 1, 2015; U.S. Provisional Patent Application Ser. No. 62/067,194, filed Oct. 22, 2014; U.S. Pat. No. 10,920,208, issued Feb. 16, 2021; International PCT Application PCT/US2018/048134, filed Aug. 27, 2018, published as WO 2019/040935; U.S. Pat. No. 9,267,127, issued Feb. 23, 2016; International PCT Application PCT Application, PCT/US2015/057012, filed Oct. 22, 2015, published as WO 2016/077052; International PCT Application PCT/US2016/027795, filed Apr. 15, 2016, published as WO 2016/168631; International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Provisional Patent Application Ser. No. 62/067,194, filed Oct. 22, 2014, U.S. Pat. No. 9,023,594, issued May 5, 2015, and International PCT Application, PCT/US2018/051557, published as WO 2019/056002 on Mar. 21, 2019, the entire contents of each of which is incorporated herein by reference.

The term “non-continuous evolution,” as used herein, also refers to an evolution procedure in which a population of nucleic acids encoding a protein of interest (e.g. protein degron) is subjected to multiple rounds of: (a) replication, (b) mutation (or modification of the primary sequence of nucleotides of the nucleic acids in the population), and (c) selection to produce a desired evolved product, for example, a novel nucleic acid encoding a novel protein with a desired activity (e.g., ability to bind with small molecule-bound CRBN).

, wherein the multiple rounds of replication, mutation, and selection require investigator intervention to move the process from one phase to another. Non-continuous evolution is similar to continuous evolution in that it uses the same selection principles, but it is performed using serial dilutions instead of under continuous flow. A non-continuous evolution process may be used as a lower stringency alternative to continuous evolution process.

The term “phage-assisted non-continuous evolution” (also used interchangeably herein with “PANCE”), as used herein, refers to non-continuous evolution that employs phage as viral vectors. The general concept of PANCE technology has been described, for example, in Miller et al. Nature Protoc. 2020 December; 15 (12): 4101-4127, and International PCT Application PCT/US2020/042016, published as WO 2021/011579, the entire contents of each of which are incorporated herein by reference. PANCE uses the same selection principles as PACE, but it is performed through serial dilution instead of under continuous flow. PANCE has a lower stringency nature than PACE due to increased time allowed for phage propagation. PANCE may be performed in multi-well plates which enables parallel evolution towards many different targets or many replicates of the same evolution.

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, 4-acetylcytidine, 5-(carboxyhydroxymethyl) uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′-N-phosphoramidite linkages).

An “isolated nucleic acid” generally refers to refers to a nucleic acid that is: (i) amplified in vitro by, for example, polymerase chain reaction (PCR); (ii) recombinantly produced by molecular cloning; (iii) purified, as by restriction endonuclease cleavage and gel electrophoretic fractionation, or column chromatography; or (iv) synthesized by, for example, chemical synthesis. An isolated nucleic acid is one which is readily manipulatable by recombinant DNA techniques known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulatable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The term “gene of interest” or “gene encoding a protein (e.g., degron) of interest,” as used herein, refers to a nucleic acid construct comprising a nucleotide sequence encoding a gene product (e.g., a protein degron) of interest (e.g., for its properties, either desirable or undesirable) to be evolved in a continuous evolution process as described herein. The term includes any variations of a gene of interest that are the result of a continuous evolution process according to methods described herein (e.g., increase expression, decreased expression, modulated or changed activity, modulated or changed specificity). For example, in some embodiments, a gene of interest is a nucleic acid construct comprising a nucleotide sequence encoding a protein degron to be evolved, cloned into a viral vector, for example, a phage genome, so that the expression of the encoding sequence is under the control of one or more promoters in the viral genome. In other embodiments, a gene of interest is a nucleic acid construct comprising a nucleotide sequence encoding a protein degron to be evolved and a promoter operably linked to the encoding sequence. When cloned into a viral vector, for example, a phage genome, the expression of the encoding sequence of such genes of interest is under the control of the heterologous promoter and, in some embodiments, may also be influenced by one or more promoters in the viral genome.

The term “vector,” as used herein, refers to any genetic element, such as a plasmid, phage, transposon, cosmid, chromosome, artificial chromosome, virus, virion, etc., which is capable of replication when associated with the proper control elements, and which can transfer gene sequences between cells.

The term “viral vector,” as used herein, refers to a nucleic acid (or isolated nucleic acid) comprising a viral genome that, when introduced into a suitable host cell, can be replicated and packaged into viral particles able to transfer the viral genome into another host cell. The term viral vector extends to vectors comprising truncated or partial viral genomes. For example, in some embodiments, a viral vector is provided that lacks a gene encoding a protein essential for the generation of infectious viral particles or for viral replication. In suitable host cells, for example, host cells comprising the lacking gene under the control of a conditional promoter, however, such truncated viral vectors can replicate and generate viral particles able to transfer the truncated viral genome into another host cell. In some embodiments, the viral vector is a phage, for example, a filamentous phage (e.g., an M13 phage). In some embodiments, a viral vector, for example, a phage vector, is provided that comprises a gene of interest to be evolved.

The term “host cell,” as used herein, refers to a cell that can host a viral vector useful for a continuous evolution process as provided herein. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. For example, if the viral vector is a modified M13 phage genome, as provided in some embodiments described herein, then a suitable host cell would be any cell that can support the wild-type M13 phage life cycle. Suitable host cells for viral vectors useful in continuous evolution processes are well known to those of skill in the art, and the invention is not limited in this respect.

In some embodiments, modified viral vectors are used in continuous evolution processes as provided herein. In some embodiments, such modified viral vectors lack a gene required for the generation of infectious viral particles. In some such embodiments, a suitable host cell is a cell comprising the gene required for the generation of infectious viral particles, for example, under the control of a constitutive or a conditional promoter (e.g., in the form of an accessory plasmid, as described herein). In some embodiments, the viral vector used lacks a plurality of viral genes. In some such embodiments, a suitable host cell is a cell that comprises a helper construct providing the viral genes required for the generation of viral particles. A cell is not required to actually support the life cycle of a viral vector used in the methods provided herein. For example, a cell comprising a gene required for the generation of infectious viral particles under the control of a conditional promoter may not support the life cycle of a viral vector that does not comprise a gene of interest able to activate the promoter, but it is still a suitable host cell for such a viral vector. In some embodiments, the viral vector is a phage, and the host cell is a bacterial cell. In some embodiments, the host cell is an E. coli cell. Suitable E. coli host strains will be apparent to those of skill in the art, and include, but are not limited to, New England Biolabs (NEB) Turbo, Top10F′, DH12S, ER2738, ER2267, XL1-Blue MRF′, and DH10B. In some embodiments, the strain of E. coli used is known as S1030 (available from Addgene). In some embodiments, the strain of E. coli use to express proteins is BL21 (DE3). These strain names are art recognized, and the genotype of these strains has been well characterized. It should be understood that the above strains are exemplary only, and that the invention is not limited in this respect.

The term “fresh,” as used herein interchangeably with the terms “non-infected” or “uninfected” in the context of host cells, refers to a host cell that has not been infected by a viral vector comprising a gene of interest as used in a continuous evolution process provided herein. A fresh host cell can, however, have been infected by a viral vector unrelated to the vector to be evolved or by a vector of the same or a similar type but not carrying the gene of interest.

The term “promoter” refers to a nucleic acid molecule with a sequence recognized by the cellular transcription machinery and able to initiate transcription of a downstream gene. A promoter can be constitutively active, meaning that the promoter is always active in a given cellular context, or conditionally active, meaning that the promoter is only active under specific conditions. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect.

The term “phage,” as used herein interchangeably with the term “bacteriophage,” refers to a virus that infects bacterial cells. Typically, phages consist of an outer protein capsid enclosing genetic material. The genetic material can be ssRNA, dsRNA, ssDNA, or dsDNA, in either linear or circular form. Phages and phage vectors are well known to those of skill in the art and non-limiting examples of phages that are useful for carrying out the methods provided herein are λ (Lysogen), T2, T4, T7, T12, R17, M13, MS2, G4, P1, P2, P4, Phi X174, N4, @6, and @29. In certain embodiments, the phage utilized in the present invention is M13. Additional suitable phages and host cells will be apparent to those of skill in the art, and the invention is not limited in this aspect. For an exemplary description of additional suitable phages and host cells, see Elizabeth Kutter and Alexander Sulakvelidze: Bacteriophages: Biology and Applications. CRC Press; 1^{st} edition (December 2004), ISBN: 0849313368; Martha R. J. Clokie and Andrew M. Kropinski: Bacteriophages: Methods and Protocols, Volume 1: Isolation, Characterization, and Interactions (Methods in Molecular Biology) Humana Press; 1^{st} edition (December 2008), ISBN: 1588296822; Martha R. J. Clokie and Andrew M. Kropinski: Bacteriophages: Methods and Protocols, Volume 2: Molecular and Applied Aspects (Methods in Molecular Biology) Humana Press; 1^{st} edition (December 2008), ISBN: 1603275649; all of which are incorporated herein in their entirety by reference for disclosure of suitable phages and host cells as well as methods and protocols for isolation, culture, and manipulation of such phages).

In some embodiments, the phage is a filamentous phage. In some embodiments, the phage is an M13 phage. M13 phages are well known to those in the art and the biology of M13 phages has extensively been studied. Wild type M13 phage particles comprise a circular, single-stranded genome of approximately 6.4 kb. In certain embodiments, the wild-type genome of an M13 phage includes eleven genes, gI-gXI, which, in turn, encode the eleven M13 proteins, pI-pXI, respectively. gVIII encodes pVIII, also often referred to as the major structural protein of the phage particles, while gIII encodes pIII, also referred to as the minor coat protein, which is required for infectivity of M13 phage particles, whereas gIII-neg encodes and antagonistic protein to pIII.

The term “selection phage,” as used herein interchangeably with the term “selection plasmid,” refers to a modified phage that comprises a gene of interest to be evolved and lacks a full-length gene encoding a protein required for the generation of infectious phage particles. For example, some M13 selection phages provided herein comprise a nucleic acid sequence encoding a protein degron to be evolved, e.g., under the control of an M13 promoter, and lack all or part of a phage gene encoding a protein required for the generation of infectious phage particles, e.g., gI, gII, gIII, gIV, gV, gVI, gVII, gVIII, gIX, or gX, or any combination thereof. For example, some M13 selection phages provided herein comprise a nucleic acid sequence encoding a protein degron to be evolved, e.g., under the control of an M13 promoter, and lack all or part of a gene encoding a protein required for the generation of infective phage particles, e.g., the gIII gene encoding the pIII protein.

The term “helper phage,” as used herein interchangeable with the terms “helper phagemid” and “helper plasmid,” refers to a nucleic acid construct comprising a phage gene required for the phage life cycle, or a plurality of such genes, but lacking a structural element required for genome packaging into a phage particle. For example, a helper phage may provide a wild-type phage genome lacking a phage origin of replication. In some embodiments, a helper phage is provided that comprises a gene required for the generation of phage particles, but lacks a gene required for the generation of infectious particles, for example, a full-length pIII gene. In some embodiments, the helper phage provides only some, but not all, genes required for the generation of phage particles. Helper phages are useful to allow modified phages that lack a gene required for the generation of phage particles to complete the phage life cycle in a host cell. Typically, a helper phage will comprise the genes required for the generation of phage particles that are lacking in the phage genome, thus complementing the phage genome. In the continuous evolution context, the helper phage typically complements the selection phage, but both lack a phage gene required for the production of infectious phage particles.

The term “replication product,” as used herein, refers to a nucleic acid that is the result of viral genome replication by a host cell. This includes any viral genomes synthesized by the host cell from a viral genome inserted into the host cell. The term includes non-mutated as well as mutated replication products.

The term “accessory plasmid,” as used herein, refers to a plasmid comprising a gene required for the generation of infectious viral particles under the control of a conditional promoter. In the context of continuous evolution described herein, the conditional promoter of the accessory plasmid is typically activated by a function of the gene of interest to be evolved. Accordingly, the accessory plasmid serves the function of conveying a competitive advantage (in the case of positive selection) to those viral vectors in a given population of viral vectors that carry a gene of interest able to activate the conditional promoter. Only viral vectors carrying an “activating” gene of interest will be able to induce expression of the gene required to generate infectious viral particles in the host cell, and, thus, allow for packaging and propagation of the viral genome in the flow of host cells. Vectors carrying non-activating versions of the gene of interest, on the other hand, will not induce expression of the gene required to generate infectious viral vectors, and, thus, will not be packaged into viral particles that can infect fresh host cells.

In some embodiments, the conditional promoter of the accessory plasmid is a promoter the transcriptional activity of which can be regulated over a wide range, for example, over 2, 3, 4, 5, 6, 7, 8, 9, or 10 orders of magnitude by the activating function, for example, function of a protein encoded by the gene of interest. In some embodiments, the level of transcriptional activity of the conditional promoter depends directly on the desired function of the gene of interest. This allows for starting a continuous evolution process with a viral vector population comprising versions of the gene of interest that only show minimal activation of the conditional promoter. In the process of continuous evolution, any mutation in the gene of interest that increases activity of the conditional promoter directly translates into higher expression levels of the gene required for the generation of infectious viral particles, and, thus, into a competitive advantage over other viral vectors carrying minimally active or loss-of-function versions of the gene of interest.

The term “mutagen,” as used herein, refers to an agent that induces mutations or increases the rate of mutation in a given biological system, for example, a host cell, to a level above the naturally-occurring level of mutation in that system. Some exemplary mutagens useful for continuous evolution procedures are provided elsewhere herein and other useful mutagens will be evident to those of skill in the art. Useful mutagens include, but are not limited to, ionizing radiation, ultraviolet radiation, base analogs, deaminating agents (e.g., nitrous acid), intercalating agents (e.g., ethidium bromide), alkylating agents (e.g., ethylnitrosourea), transposons, bromine, azide salts, psoralen, benzene, 3-Chloro-4-(dichloromethyl)-5-hydroxy-2(5H)-furanone (MX) (CAS no. 77439-76-0), O,O-dimethyl-S-(phthalimidomethyl)phosphorodithioate (phos-met) (CAS no. 732-11-6), formaldehyde (CAS no. 50-00-0), 2-(2-furyl)-3-(5-nitro-2-furyl) acrylamide (AF-2) (CAS no. 3688-53-7), glyoxal (CAS no. 107-22-2), 6-mercaptopurine (CAS no. 50-44-2), N-(trichloromethylthio)-4-cyclohexane-1,2-dicarboximide (captan) (CAS no. 133-06-2), 2-aminopurine (CAS no. 452-06-2), methyl methane sulfonate (MMS) (CAS No. 66-27-3), 4-nitroquinoline 1-oxide (4-NQO) (CAS No. 56-57-5), N4-Aminocytidine (CAS no. 57294-74-3), sodium azide (CAS no. 26628-22-8), N-ethyl-N-nitrosourea (ENU) (CAS no. 759-73-9), N-methyl-N-nitrosourea (MNU) (CAS no. 820-60-0), 5-azacytidine (CAS no. 320-67-2), cumene hydroperoxide (CHP) (CAS no. 80-15-9), ethyl methanesulfonate (EMS) (CAS no. 62-50-0), N-ethyl-N-nitro-N-nitrosoguanidine (ENNG) (CAS no. 4245-77-6), N-methyl-N-nitro-N-nitrosoguanidine (MNNG) (CAS no. 70-25-7), 5-diazouracil (CAS no. 2435-76-9) and t-butyl hydroperoxide (BHP) (CAS no. 75-91-2). Additional mutagens can be used in continuous evolution procedures as provided herein, and the invention is not limited in this respect.

Ideally, a mutagen is used at a concentration or level of exposure that induces a desired mutation rate in a given host cell or viral vector population, but is not significantly toxic to the host cells used within the average time frame a host cell is exposed to the mutagen or the time a host cell is present in the host cell flow before being replaced by a fresh host cell.

The term “mutagenesis plasmid,” as used herein, refers to a plasmid comprising a gene encoding a gene product that acts as a mutagen. In some embodiments, the gene encodes a DNA polymerase lacking a proofreading capability. In some embodiments, the gene is a gene involved in the bacterial SOS stress response, for example, a UmuC, UmuD′, or RecA gene. In some embodiments, the gene is a GATC methylase gene, for example, a deoxyadenosine methylase (dam methylase) gene. In some embodiments, the gene is involved in binding of hemimethylated GATC sequences, for example, a seqA gene. In some embodiments, the gene is involved with repression of mutagenic nucleobase export, for example emrR. In some embodiments, the gene is involved with inhibition of uracil DNA-glycosylase, for example a Uracil Glycosylase Inhibitor (ugi) gene. In some embodiments, the gene is involved with deamination of cytidine (e.g., a cytidine deaminase from Petromyzon marinus), for example, cytidine deaminase 1 (CDA1). In some embodiments, the mutagenesis-promoting gene is under the control of an inducible promoter. In some embodiments, a bacterial host cell population is provided in which the host cells comprise a mutagenesis plasmid in which a dnaQ926, UmuC, UmuD′, and RecA expression cassette is controlled by an arabinose-inducible promoter. In some such embodiments, the population of host cells is contacted with the inducer, for example, arabinose in an amount sufficient to induce an increased rate of mutation. In some embodiments, the mutagenesis plasmid is an MP4 mutagenesis plasmid or an MP6 mutagenesis plasmid. The MP4 and MP6 mutagenesis plasmids are described, for example in PCT Application PCT/US2016/27795, published as WO 2016/168631 on Oct. 20, 2016, the content of which is incorporated herein in its entirety. The MP4 mutagenesis plasmid comprises the following genes: dnaQ926, dam, seqA¹⁷. The MP6 mutagenesis plasmid comprises the following genes: dnaQ926, dam, seqA, emrR, Ugi, and CDA1²².

The term “cell,” as used herein, refers to a cell derived from an individual organism, for example, from a mammal. A cell may be a prokaryotic cell or a eukaryotic cell. In some embodiments, the cell is a eukaryotic cell, for example, a human cell, a mouse cell, a dog cell, a cat cell, a horse cell, a guinea pig cell, a pig cell, a hamster cell, a non-human primate (e.g. monkey) cell, etc. In some embodiments, the cell is in a subject (e.g., the cell is in vivo). In some embodiments, the cell is intact (e.g., the outer membrane of the cell, such as the plasma membrane, is intact or not permeabilized).

The term “intracellular environment,” as used herein, refers to the aqueous biological fluid (e.g., cytosol or cytoplasm) forming the microenvironment contained by the outer membrane of a cell. For example, in a subject, an intracellular environment may include the cytoplasm of a cell or cells of a target organ or tissue (e.g., the nucleoplasm of the nucleus of a cell). In another example, a cellular environment is the cytoplasm of a cell or cells surrounded by cell culture growth media housed in an in vitro culture vessel, such as a cell culture plate or flask.

The “percent identity” of two amino acid sequences may be determined using algorithms or computer programs, for example, the algorithm of Karlin and Altschul, Proc. Natl. Acad. Sci. USA 87:2264-68, 1990, modified as in Karlin and Altschul, Proc. Natl. Acad. Sci. USA 90:5873-77, 1993. Such an algorithm is incorporated into various computer programs, for example NBLAST and XBLAST programs (version 2.0) of Altschul et al., J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25 (17): 3389-3402, 1997. When utilizing BLAST and Gapped BLAST programs, the default parameters of the respective programs (e.g., XBLAST and NBLAST) can be used. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul, S F et al., (1997) Nuc. Acids Res. 25:3389-3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

### DETAILED DESCRIPTION

Aspects of the disclosure relate to compositions, methods, systems, and uses for small-molecule-inducible tagged protein degradation. Using a super degron starting sequence, SD0 (SEQ ID NO: 1), evolved protein degrons were developed to form a strong ternary complex with small molecule (e.g., PT-179, PK-1016, and VS-777) bound cereblon (CRBN). The evolved protein degrons provided herein serve as potent, small molecule (e.g., PT-179, PK-1016, and VS-777) responsive degron tags. In some embodiments, the evolved protein degrons are the basis for new molecular glue ternary interactions. For example, inclusion of an evolved protein degrons described herein on a target protein (e.g., therapeutic protein) is advantageous because it provides an “off” switch by which the level or activity of the recombinant protein may be regulated (e.g., reduced by protein degradation) using a small molecule CRBN substrate.

Some aspects of this disclosure are based on the recognition that certain directed evolution technologies, for example, PACE and PANCE, can be employed to create evolved protein degron variants. The evolution includes positive and negative selection systems that bias evolution of a super degron towards production of evolved degrons variants. Protein degrons may require many successive mutations to remodel complex networks of contacts with polypeptide substrates and are thus not readily manipulated by conventional, iterative evolution methods. Continuous evolution strategies, which require little or no researcher intervention between generations, therefore are well-suited to evolve protein degrons capable of forming new ternary complexes used for small molecule inducible tagged protein degradation, that differs substantially in sequence from the starting super degron sequence.

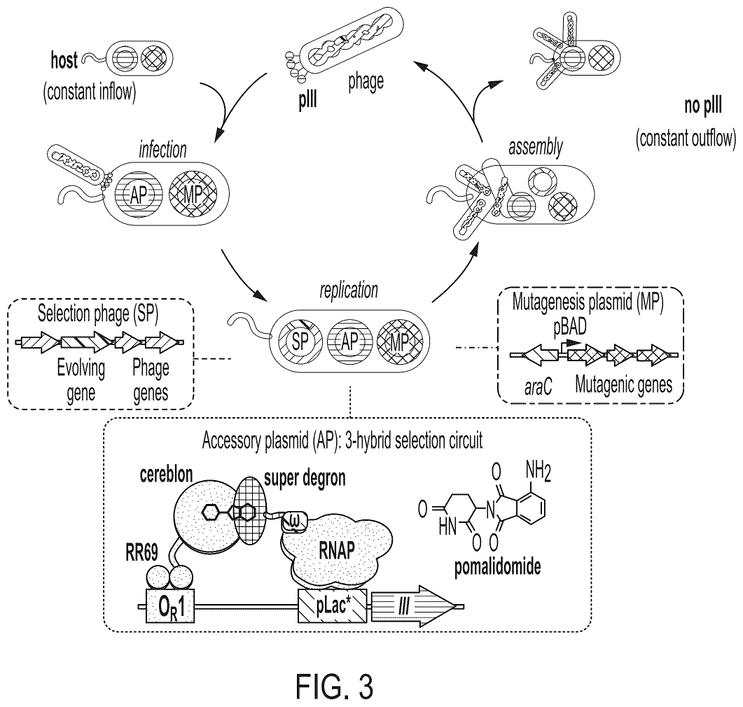

The ability of PACE to perform the equivalent of hundreds of rounds of iterative evolution methods within days enables complex degron evolution experiments, that are impractical with conventional methods. This disclosure provides data demonstrating the use of PACE evolution to evolve super degrons (e.g., SD0, SEQ ID NO: 1) into degrons that form a strong ternary complex with small molecule-bound cereblon. In some embodiments, provided herein are evolved degrons for small-molecule-inducible tagged protein degradation. In some embodiments, the small molecule is VS-777, PT-179, or PK-1016, shown below and in FIG. 3. In some embodiments, the small molecule is PT-179.

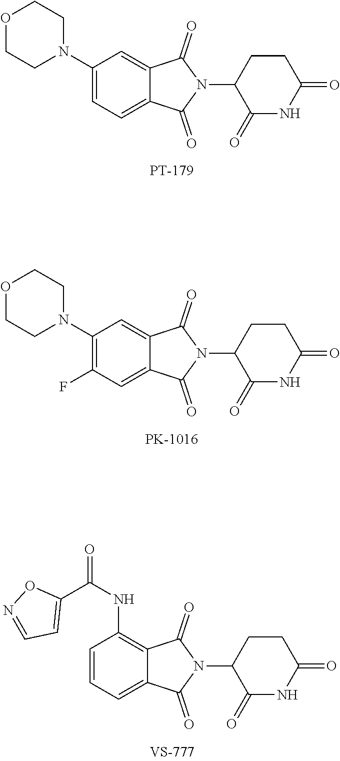 [SMILES: O=C1CCC(N2C(=O)c3ccc(N4CCOCC4)cc3C2=O)C(=O)N1 ; O=C1CCC(N2C(=O)c3cc(F)c(N4CCOCC4)cc3C2=O)C(=O)N1 ; O=C1CCC(N2C(=O)c3cccc(NC(=O)c4ccno4)c3C2=O)C(=O)N1]

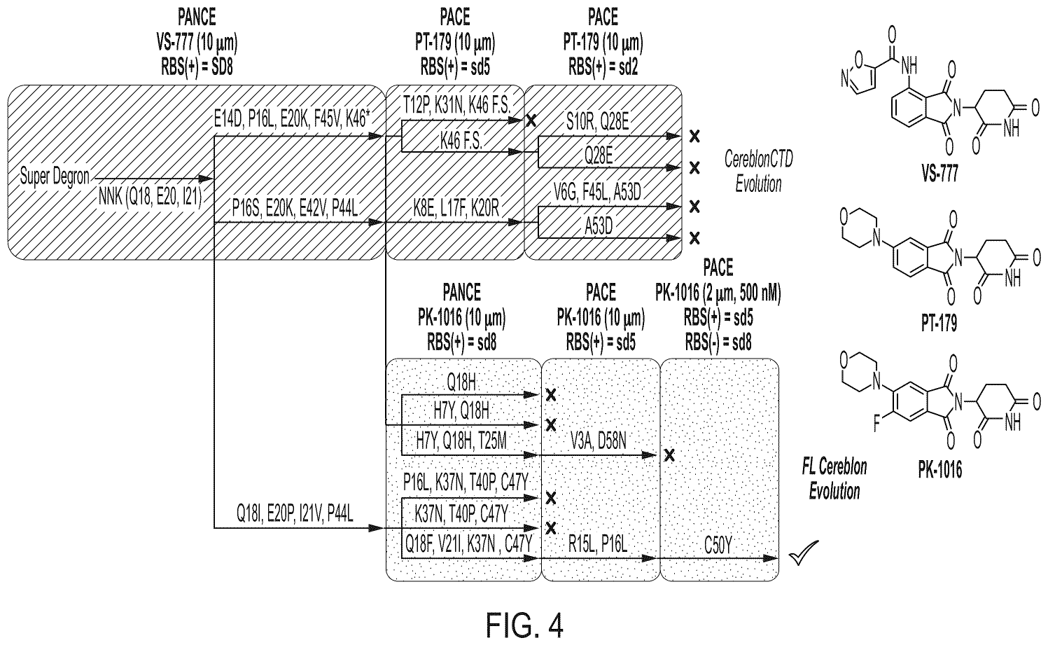

As described in the Examples, super degron, SD0 (SEQ ID NO: 1), which normally forms a ternary complex with IMiD-bound cereblon, was first evolved by PACE to form a ternary complex with VS-777-bound cereblon. Further iterative evolutions using PACE to form ternary complexes with PT-179-bound cereblon and PK-1016-bound cereblon produced a series of evolutionary intermediates (as shown in FIG. 4) and it was observed that the resulting protein degron variants contain up to 13 amino acid substitutions relative to the 60 amino acid long starting super degron sequence, SD0 (SEQ ID NO: 1). The work described herein provides novel protein degrons resulting from directed evolution to engage small molecule (e.g., PT-179, PK-1016, and VS-777) bound cereblon, and novel methods for degrading a target protein in a cell using the evolved protein degrons.

In phage-assisted continuous evolution (PACE), a population of evolving selection phage (SP) is continuously diluted in a fixed-volume vessel by an incoming culture of host cells, e.g., E. coli. The SP is a modified phage genome in which the evolving gene of interest (e.g. gene encoding a protein degron) has replaced gene III (gIII), a gene essential for phage infectivity. If the evolving gene of interest (e.g. gene encoding a protein degron) possesses the desired activity (e.g., ability to bind with small molecule-bound CRBN), it will trigger expression of gene III from an accessory plasmid (AP) in the host cell, thus producing infectious progeny encoding active variants of the evolving gene. The mutation rate of the SP is controlled using an inducible mutagenesis plasmid (MP), such as MP6, which upon induction increases the mutation rate of the SP by >300,000-fold. Because the rate of continuous dilution is slower than phage replication but faster than E. coli replication, mutations only accumulate in the SP.

PACE technology has been described previously, for example, in U.S. Pat. No. 9,023,594, issued May 5, 2015; U.S. Pat. No. 9,771,574, issued Sep. 26, 2017; U.S. patent application Ser. No. 15/713,403, filed Sep. 22, 2017 (now abandoned); International PCT Application PCT/US2009/056194, filed Sep. 8, 2009, published as WO 2010/028347 on Mar. 11, 2010; U.S. Provisional Patent Application Ser. No. 61/426,139, filed Dec. 22, 2010; U.S. Pat. No. 9,394,537, issued Jul. 19, 2016; U.S. Pat. No. 10,336,997, issued Jul. 2, 2019; U.S. Pat. No. 11,214,792, issued Jan. 4, 2022; International PCT Application PCT/US2011/066747, filed Dec. 22, 2011, published as WO 2012/088381 on Jun. 28, 2012; U.S. Provisional Patent Application Ser. No. 61/929,378 filed Jan. 20, 2014; U.S. Pat. No. 10,179,911, issued Jan. 15, 2019; U.S. patent application Ser. No. 16/238,386, filed Jan. 2, 2019; International PCT Application PCT/US2015/012022, filed Jan. 20, 2015; U.S. Provisional Patent Application Ser. No. 62/158,982, filed May 8, 2015; U.S. Provisional Patent Application Ser. No. 62/187,669, filed Jul. 1, 2015; U.S. Provisional Patent Application Ser. No. 62/067,194, filed Oct. 22, 2014; U.S. Pat. No. 10,920,208, issued Feb. 16, 2021; International PCT Application PCT/US2018/048134, filed Aug. 27, 2018, published as WO 2019/040935; U.S. Pat. No. 9,267,127, issued Feb. 23, 2016; International PCT Application PCT Application, PCT/US2015/057012, filed Oct. 22, 2015, published as WO 2016/077052; International PCT Application PCT/US2016/027795, filed Apr. 15, 2016, published as WO 2016/168631; International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Provisional Patent Application Ser. No. 62/067,194, filed Oct. 22, 2014, U.S. Pat. No. 9,023,594, issued May 5, 2015, and International PCT Application, PCT/US2018/051557, published as WO 2019/056002 on Mar. 21, 2019, the entire contents of each of which is incorporated herein by reference.

The PACE system may also be adapted into the format of PANCE (phage-assisted non-continuous evolution), a non-continuous form of PACE in which cultures propagate phage in wells through multiple generations but undergo serial daily passaging in lieu of continuous flow, permitting a less stringent and more sensitive initial selection. PANCE has been described previously, for example, in Miller et al. Nature Protoc 2020 December; 15 (12): 4101-4127, and International PCT Application PCT/US2020/042016, published as WO 2021/011579, the entire contents of each of which are incorporated herein by reference, the entire contents of each of which are incorporated herein by reference.

### Degron Variants

Protein degron variants disclosed herein are evolved from a super degron to form a strong ternary complex with small molecule (e.g., VS-777, PK-1016, or PT-179) bound-CRBN, for small molecule inducible tagged protein degradation. Provided herein are evolved protein degrons with increased sensitivity to small molecule-bound CRBN, wherein the small molecule bound to CRBN is a small molecule other than thalidomide and/or other therapeutic IMiDs, and thus are the basis for new molecular glue ternary interactions. In some embodiments, the evolved degrons provided herein serve as a potent, small molecule (e.g., VS-777, PK-1016, or PT-179) responsive degron tags. In some embodiments, the evolved degrons are more selective than natural degrons because they respond to a small molecule that has much less biological cross-talk than the canonical small-molecule triggers thalidomide and other therapeutic IMiDs. In some embodiments, the small molecule is VS-777. In some embodiments, the small molecule is PK-1016. In some embodiments, the small molecule is PT-179.

In some embodiments, the evolved protein degrons have one or more amino acid variations introduced into the amino acid sequence, e.g., as a result of application of the PACE/PANCE methods or by genetic engineering, as compared to the amino acid sequence of the starting degron (e.g., super degron of SEQ ID NO: 1). Amino acid sequence variations may include one or more (e.g., 2, 3, 4, 5, 6, 7, 8, 9, 10, 11, 12, 13, 14, 15, 16, 17, 18, 19, 20, or more) mutated residues within the amino acid sequence of the degron, e.g., as a result of a substitution of one amino acid for another, the deletion of one or more amino acids (e.g., a truncated protein), the insertion of one or more amino acids, or any combination of the foregoing.

In some embodiments, a protein degron is evolved by phage-assisted continuous evolution (PACE) and/or phage-assisted non-continuous evolution (PANCE). In some embodiments, an evolved protein degron requires many generations (e.g., 2, 3, 4, 5, 6, 7, 8, 9, 10 or more generations) of evolution. In some embodiments, the disclosure provides variants of protein degrons that are derived from a super degron sequence (SEQ ID NO.: 1) and have at least one amino acid variation in at least one of the positions recited in Table 1 or Table 2. In some embodiments, the disclosure provides variants of protein degrons that are derived from a super degron sequence (SEQ ID NO.: 1) and have at least one amino acid variation in at least one of the positions selected from F1, V3, M5, V6, H7, K8, S10, T12, E14, R15, P16, L17, Q18, E20, 121, T25, Q28, K29, G30, N31, K37, T40, G41, E42, P44, F45, K46, C47, C50, N51, A53, C54, R57, D58, A59, and L60 relative to SEQ ID NO: 1.

The variation in amino acid sequence generally results from a mutation, insertion, or deletion in a DNA coding sequence. In some embodiments, mutation of a DNA sequence results in a non-synonymous (i.e., conservative, semi-conservative, or radical) amino acid substitution. In some embodiments an insertion or deletion is an “in-frame” insertion or deletion that does not alter the reading frame the resulting mutant protein.

The amount or level of variation between the super degron (SEQ ID NO: 1) and a protein degron variant provided herein can be expressed as the percent identity of the nucleic acid sequences or amino acid sequences between the two genes or proteins, respectively.

In some embodiments, the amount of variation between the super degron (SEQ ID NO: 1) and a protein degron variant is expressed as the percent identity at the amino acid sequence level. In some embodiments, a protein degron variant is from about 50%, about 60% to about 99.9% identical, about 70% to about 98% identical, about 75% to about 95% identical, about 80% to about 90% identical, about 85% to about 95% identical, or about 95% to about 99% identical to the sequence set forth in SEQ ID NO: 1. In some embodiments, a protein degron variant comprises an amino acid sequence that is at least 50% identical to the sequence set forth in SEQ ID NO: 1. In some embodiments, a protein degron variant comprises an amino acid sequence that is at least 60% identical to the sequence set forth in SEQ ID NO: 1. In some embodiments, a protein degron variant comprises an amino acid sequence that is at least 60%, 65%, 70%, 75%, 80%, 85%, 90%, 95%, 98% or 99% identical to the sequence set forth in SEQ ID NO: 1. In some embodiments, a protein degron variant comprises an amino acid sequence that is at least 80%, 85%, 90%, 95%, 98% or 99% identical to the sequence set forth in SEQ ID NO: 1.

In some embodiments, a protein degron variant comprises an amino acid sequence that is at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.9% identical to the sequence set forth in SEQ ID NO: 1. In some embodiments, a protein degron variant comprises an amino acid sequence that is at least 80%, 95%, 90%, 95%, or 99% identical to the amino acid sequence set forth in SEQ ID NO: 1. In some embodiments, a protein degron variant comprises an amino acid sequence that is at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.9% identical to the sequence set forth in SEQ ID NO: 1 and comprises an amino acid substitution at one or more positions recited in Table 1 or Table 2. In some embodiments, a protein degron variant comprises an amino acid sequence that is at least 80%, 95%, 90%, 95%, or 99% identical to the amino acid sequence set forth in SEQ ID NO: 1 and comprises an amino acid substitution at one or more positions recited in Table 1 or Table 2. In some embodiments, a protein degron variant comprises an amino acid sequence that is at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.9% identical to the sequence set forth in SEQ ID NO: 1 and comprises an amino acid substitution at one or more of the following positions: F1, V3, M5, V6, H7, K8, S10, T12, E14, R15, P16, L17, Q18, E20, 121, T25, Q28, K29, G30, N31, K37, T40, G41, E42, P44, F45, K46, C47, C50, N51, A53, C54, R57, D58, A59, and L60. In some embodiments, a protein degron variant comprises an amino acid sequence that is at least 80%, 95%, 90%, 95%, or 99% identical to the amino acid sequence set forth in SEQ ID NO: 1 and comprises an amino acid substitution at one or more of the following positions: F1, V3, M5, V6, H7, K8, S10, T12, E14, R15, P16, L17, Q18, E20, 121, T25, Q28, K29, G30, N31, K37, T40, G41, E42, P44, F45, K46, C47, C50, N51, A53, C54, R57, D58, A59, and L60.

Some aspects of the disclosure provide protein degron variants comprising an amino acid sequence having between about 80% and about 99.9% (e.g., about 80%, about 80.5%, about 81%, about 81.5%, about 82%, about 82.5%, about 83%, about 83.5%, about 84%, about 84.5%, about 85%, about 85.5%, about 86%, about 86.5%, about 87%, about 87.5%, about 88%, about 88.5%, about 89%, about 89.5%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.2%, about 99.4%, about 99.6%, about 99.8%, or about 99.9%) identity to the sequence set forth in SEQ ID NO: 1. In some embodiments, a protein degron comprises an amino acid sequence having between about 80% and about 99.9% (e.g., about 80%, about 80.5%, about 81%, about 81.5%, about 82%, about 82.5%, about 83%, about 83.5%, about 84%, about 84.5%, about 85%, about 85.5%, about 86%, about 86.5%, about 87%, about 87.5%, about 88%, about 88.5%, about 89%, about 89.5%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.2%, about 99.4%, about 99.6%, about 99.8%, or about 99.9%) identity to the sequence set forth in SEQ ID NO: 1 and comprises an amino acid substitution at one or more of the following positions: F1, V3, M5, V6, H7, K8, S10, T12, E14, R15, P16, L17, Q18, E20, 121, T25, Q28, K29, G30, N31, K37, T40, G41, E42, P44, F45, K46, C47, C50, N51, A53, C54, R57, D58, A59, and L60. In some embodiments, the protein degron is no more than 99.9% identical to the sequence set forth in SEQ ID NO: 1.

Some aspects of the disclosure provide protein degron variants having between 1 and 15 amino acid substitutions (e.g., mutations) relative to SEQ ID NO: 1 (e.g., 1, 2, 3, 4, 5, etc.). Some aspects of the disclosure provide protein degron variants having more than 15 amino acid substitutions (e.g., mutations) relative to SEQ ID NO: 1 (e.g., 20, 25, 30, 40, etc.). In some embodiments, a protein degron variant has 2, 3, 4, 5, 6, 7, 8, 9, 10, 11, 12, 13, 14, or 15 amino acid substitutions relative to a SEQ ID NO: 1. The mutations disclosed herein are not exclusive of other mutations which may occur or be introduced. For example, a protein degron variant may have a mutation as described herein in addition to at least one mutation not described herein (e.g., 1, 2, 3, 4, 5, etc. additional mutations).

In some embodiments, a protein degron variant comprises one or more amino acid substitutions at a position selected from F1, V3, M5, V6, H7, K8, S10, T12, E14, R15, P16, L17, Q18, E20, 121, T25, Q28, K29, G30, N31, K37, T40, G41, E42, P44, F45, K46, C47, C50, N51, A53, C54, R57, D58, A59, and L60 relative to SEQ ID NO: 1. In some embodiments, a protein degron variant comprises one or more amino acid substitutions selected from FIL, V3E, V3A, M5L, V6G, H7Y, K8E, K8R, S10R, T12P, E14D, R15L, P16S, P16L, L17F, Q18M, Q18I, Q18H, Q18F, E20K, E20P, E20R, I21V, T25M, Q28E, Q28K, K29E, G30V, N31K, N31D, N31T, K37N, T40M, T40P, G41D, E42V, P44L, P44T, P44M, F45V, F45L, K46R, K46stop, C47Y, C50Y, C50R, N51K, N51H, A53D, C54Y, R57K, D58R, D58N, A59C, and L60F relative to SEQ ID NO: 1. In some embodiments, a protein degron variant comprises the following amino acid substitutions relative to SEQ ID NO: 1: R15L, P16L, Q18F, E20P, K37N, P44L, C47Y, and C50Y.

In some embodiments, a protein degron variant comprises the following amino acid substitutions relative to SEQ ID NO: 1: E20K and P44L. In some embodiments, a protein degron variant comprises the following amino acid substitutions relative to SEQ ID NO: 1: P16S, E20K, and P44L. In some embodiments, a protein degron variant comprises the following amino acid substitutions relative to SEQ ID NO:1: P16S, E20K, E42V, and P44L. In some embodiments, a protein degron variant comprises the following amino acid substitutions relative to SEQ ID NO: 1: M5L, P16S, E20K, E42V, and P44L. In some embodiments, a protein degron variant comprises the following amino acid substitutions relative to SEQ ID NO: 1: L17F, E20K, N31K, and P44L. In some embodiments, a protein degron variant comprises the following amino acid substitutions relative to SEQ ID NO: 1: Q18M, E20P, I21V, T40M, and P44L. In some embodiments, a protein degron variant comprises the following amino acid substitutions relative to SEQ ID NO: 1: Q18M, E20P, I21V, N31K, T40M, and P44L. In some embodiments, a protein degron variant comprises the following amino acid substitutions relative to SEQ ID NO: 1: Q18I, E20P, 121V, and P44L. In some embodiments, a protein degron variant comprises the following amino acid substitutions relative to SEQ ID NO: 1: Q18I, E20P, I21V, P44L, and N51K. In some embodiments, a protein degron variant comprises the following amino acid substitutions relative to SEQ ID NO: 1: Q18I, E20P, I21V, G30V, and P44L. In some embodiments, a protein degron variant comprises the following amino acid substitutions relative to SEQ ID NO: 1: P44T, F45V, and K46stop. In some embodiments, a protein degron variant comprises the following amino acid substitutions relative to SEQ ID NO: 1: E14D, P16L, E20K, P44T, F45V, and K46stop. In some embodiments, a protein degron variant comprises the following amino acid substitutions relative to SEQ ID NO: 1: P16L and E20K. In some embodiments, a protein degron variant comprises the following amino acid substitutions relative to SEQ ID NO: 1: V3E, P16L, and E20K. In some embodiments, a protein degron variant comprises the following amino acid substitutions relative to SEQ ID NO: 1: P16S, L17F, E20R, E42V, and P44L. In some embodiments, a protein degron variant comprises the following amino acid substitutions relative to SEQ ID NO: 1: T12P, E14D, P16L, E20K, N31D, E42V, P44T, F45V, R57K, D58R, A59C, L60F, and *61VI. In some embodiments, a protein degron variant comprises the following amino acid substitutions relative to SEQ ID NO: 1: E14D, P16L, E20K, E42V, P44T, F45V, R57K, D58R, A59C, L60F, and *61VI. In some embodiments, a protein degron variant comprises the following amino acid substitutions relative to SEQ ID NO: 1: V6G, K8E, P16S, L17F, E20R, G41D, E42V, P44L, F45L, and A53D. In some embodiments, a protein degron variant comprises the following amino acid substitutions relative to SEQ ID NO: 1: K8E, P16S, L17F, E20R, G41D, E42V, and P44L. In some embodiments, a protein degron variant comprises the following amino acid substitutions relative to SEQ ID NO: 1: S10R, E14D, P16L, E20K, Q28E, E42V, P44T, F45V, R57K, D58R, A59C, L60F, and *61VI. In some embodiments, a protein degron variant comprises the following amino acid substitutions relative to SEQ ID NO: 1: E14D, P16L, E20K, Q28E, E42V, P44T, F45V, R57K, D58R, A59C, L60F, and *61VI. In some embodiments, a protein degron variant comprises the following amino acid substitutions relative to SEQ ID NO: 1: K8E, P16S, L17F, E20R, G41D, E42V, P44L, and A53D. In some embodiments, a protein degron variant comprises the following amino acid substitutions relative to SEQ ID NO: 1: P16S, L17F, E20R, G41D, E42V, and P44L. In some embodiments, a protein degron variant comprises the following amino acid substitutions relative to SEQ ID NO: 1: L17F, E20R, G41D, E42V, and P44L. In some embodiments, a protein degron variant comprises the following amino acid substitutions relative to SEQ ID NO: 1: P16L, E20K, Q28E, E42V, P44T, F45V, R57K, D58R, A59C, L60F, and *61VI. In some embodiments, a protein degron variant comprises the following amino acid substitutions relative to SEQ ID NO: 1: S10R, E14D, P16L, E20K, Q28E, E42V, P44T, and F45V. In some embodiments, a protein degron variant comprises the following amino acid substitutions relative to SEQ ID NO: 1: P16L, E20K, Q28E, E42V, P44T, and F45V. In some embodiments, a protein degron variant comprises the following amino acid substitutions relative to SEQ ID NO: 1: P16S, Q18H, E20K, E42V, and P44L. In some embodiments, a protein degron variant comprises the following amino acid substitutions relative to SEQ ID NO: 1: H7Y, P16S, Q18H, E20K, E42V, and P44L. In some embodiments, a protein degron variant comprises the following amino acid substitutions relative to SEQ ID NO: 1: H7Y, P16S, Q18H, E20K, T25M, E42V, and P44L. In some embodiments, a protein degron variant comprises the following amino acid substitutions relative to SEQ ID NO: 1: Q18F, E20P, K37N, P44L, and C47Y. In some embodiments, a protein degron variant comprises the following amino acid substitutions relative to SEQ ID NO: 1: Q18I, E20P, I21V, K37N, T40P, P44L, and C47Y. In some embodiments, a protein degron variant comprises the following amino acid substitutions relative to SEQ ID NO: 1: P16L, Q18I, E20P, I21V, K37N, T40P, P44L, and C47Y. In some embodiments, a protein degron variant comprises the following amino acid substitutions relative to SEQ ID NO: 1: V3A, H7Y, P16S, Q18H, E20K, T25M, E42V, P44L, and D58N. In some embodiments, a protein degron variant comprises the following amino acid substitutions relative to SEQ ID NO: 1: R15L, P16L, Q18F, E20P, K37N, P44L, and C47Y. In some embodiments, a protein degron variant comprises the following amino acid substitutions relative to SEQ ID NO: 1: R15L, P16L, Q18F, E20P, K37N, P44L, C47Y, and C50Y. In some embodiments, a protein degron variant comprises the following amino acid substitutions relative to SEQ ID NO: 1: P16L, Q18F, E20P, K37N, P44L, C47Y, and C50Y. In some embodiments, a protein degron variant comprises the following amino acid substitutions relative to SEQ ID NO: 1: R15L, Q18F, E20P, K37N, P44L, C47Y, and C50Y. In some embodiments, a protein degron variant comprises the following amino acid substitutions relative to SEQ ID NO: 1: R15L, P16L, E20P, K37N, P44L, C47Y, and C50Y. In some embodiments, a protein degron variant comprises the following amino acid substitutions relative to SEQ ID NO: 1: R15L, P16L, Q18F, K37N, P44L, C47Y, and C50Y. In some embodiments, a protein degron variant comprises the following amino acid substitutions relative to SEQ ID NO: 1: R15L, P16L, Q18F, E20P, P44L, C47Y, and C50Y. In some embodiments, a protein degron variant comprises the following amino acid substitutions relative to SEQ ID NO: 1: R15L, P16L, Q18F, E20P, K37N, C47Y, and C50Y. In some embodiments, a protein degron variant comprises the following amino acid substitutions relative to SEQ ID NO: 1: R15L, P16L, Q18F, E20P, K37N, P44L, and C50Y. In some embodiments, a protein degron variant comprises the following amino acid substitutions relative to SEQ ID NO: 1: R15L, P16L, Q18F, E20P, K37N, and P44L. In some embodiments, a protein degron variant comprises the following amino acid substitutions relative to SEQ ID NO: 1: R15L, P16L, Q18F, E20P, K37N, T40P, P44L, C47Y, and C50Y. In some embodiments, a protein degron variant comprises the following amino acid substitutions relative to SEQ ID NO: 1: R15L, P16L, Q18F, E20P, Q28K, K37N, T40P, P44L, C47Y, and C50Y. In some embodiments, a protein degron variant comprises the following amino acid substitutions relative to SEQ ID NO: 1: R15L, P16L, Q18F, E20P, K37N, T40P, P44M, C47Y, C50Y, and C54Y. In some embodiments, a protein degron variant comprises the following amino acid substitutions relative to SEQ ID NO: 1: V6G, E14D, R15L, P16L, Q18F, E20P, K29E, K37N, T40P, P44L, C47Y, and C50R. In some embodiments, a protein degron variant comprises the following amino acid substitutions relative to SEQ ID NO: 1: FIL, K8R, R15L, P16L, Q18F, E20P, K37N, T40P, P44L, C47Y, C50Y, and N51H. In some embodiments, a protein degron variant comprises the following amino acid substitutions relative to SEQ ID NO 1: R15L, P16L, Q18F, E20P, K37N, T40P, P44L, K46R, C47Y, and C50Y. In some embodiments, a protein degron variant comprises the following amino acid substitutions relative to SEQ ID NO: 1: R15L, P16L, Q18F, E20P, N31T, K37N, T40P, P44L, K46R, C47Y, and C50Y.

In some embodiments, a protein degron variant has at least 70% sequence identity to (e.g., at least 70%, at least 80%, at least 85%, at least 90%, at least 95%, at least 96%, at least 97%, at least 98%, at least 99%, at least 99.5%, at least 99.9%, or more identity) a sequence selected from SEQ ID NOs.: 2-45 or 54-58. In some embodiments, a protein degron variant has at least 70% sequence identity to (e.g., at least 70%, at least 80%, at least 85%, at least 90%, at least 95%, at least 96%, at least 97%, at least 98%, at least 99%, at least 99.5%, at least 99.9%, or more identity) a sequence selected from SEQ ID NOs.: 124. In some embodiments, a protein degron variant has at least 70% sequence identity to (e.g., at least 70%, at least 80%, at least 85%, at least 90%, at least 95%, at least 96%, at least 97%, at least 98%, at least 99%, at least 99.5%, at least 99.9%, or more identity) a sequence selected from SEQ ID NOs.: 125. In some embodiments, a protein degron variant comprises or consists of an amino acid sequence set forth in any one of SEQ ID NOs.: 2-45 or 54-58. In some embodiments, a protein degron variant comprises or consists of an amino acid sequence set forth in SEQ ID NO: 37. In some embodiments, a protein degron variant comprises or consists of an amino acid sequence set forth in SEQ ID NO: 124. In some embodiments, a protein degron variant comprises or consists of an amino acid sequence set forth in SEQ ID NO: 125.

In some embodiments, the disclosure provides truncated variants of protein degrons that are derived from a super degron sequence (SEQ ID NO.: 1) and have at least one amino acid variation in at least one of the positions recited in Table 1 or Table 2. In some embodiments, the disclosure provides truncated variants of protein degrons that are derived from a super degron sequence (SEQ ID NO.: 1) and have at least one amino acid variation in at least one of the positions selected from R15, P16, Q18, E20, K37, P44, C47, and C50 relative to SEQ ID NO: 1. The variation in amino acid sequence generally results from a mutation, insertion, or deletion in a DNA coding sequence. In some embodiments, mutation of a DNA sequence results in a non-synonymous (i.e., conservative, semi-conservative, or radical) amino acid substitution. In some embodiments an insertion or deletion is an “in-frame” insertion or deletion that does not alter the reading frame of the resulting mutant protein.

The amount or level of variation between the super degron (SEQ ID NO: 1) and a truncated protein degron variant provided herein can be expressed as the percent identity of the nucleic acid sequences or amino acid sequences between the two genes or proteins, respectively.

In some embodiments, the amount of variation between the super degron (SEQ ID NO: 1) and a protein degron variant is expressed as the percent identity at the amino acid sequence level. In some embodiments, a truncated protein degron variant is from about 50% to about 99.9% identical, about 60% to about 99% identical, about 70% to about 98% identical, about 75% to about 95% identical, about 80% to about 90% identical, about 85% to about 95% identical, or about 95% to about 99% identical to amino acid residues 15-50 of SEQ ID NO: 1. In some embodiments, a truncated protein degron variant is from about 50% to about 99.9% identical, about 60% to about 99% identical, about 70% to about 98% identical, about 75% to about 95% identical, about 80% to about 90% identical, about 85% to about 95% identical, or about 95% to about 99% identical to amino acid residues 15-50 of SEQ ID NO: 1 and comprises one or more amino acid substitutions at one or more positions selected from R15, P16, Q18, E20, K37, P44, C47, and C50 relative to SEQ ID NO: 1. In some embodiments, a truncated protein degron variant comprises an amino acid sequence that is at least 50%, 55%, 60%, 65%, 70%, 75%, 80%, 85%, 90%, 95%, 98%, 99%, or 99.9% identical to amino acid residues 15-50 of SEQ ID NO: 1. In some embodiments, a truncated protein degron variant comprises an amino acid sequence that is at least 50%, 55%, 60%, 65%, 70%, 75%, 80%, 85%, 90%, 95%, 98%, 99%, or 99.9% identical to amino acid residues 15-50 of SEQ ID NO: 1 and comprises one or more amino acid substitutions at one or more positions selected from R15, P16, Q18, E20, K37, P44, C47, and C50 relative to SEQ ID NO: 1. In some embodiments, a truncated protein degron variant comprises an amino acid sequence that is at least 60% identical to amino acid residues 15-50 of SEQ ID NO: 1. In some embodiments, a truncated protein degron variant comprises an amino acid sequence that is at least 60% identical to amino acid residues 15-50 of SEQ ID NO: 1 and comprises one or more amino acid substitutions at one or more positions selected from R15, P16, Q18, E20, K37, P44, C47, and C50 relative to SEQ ID NO: 1. In some embodiments, a truncated protein degron variant comprises an amino acid sequence that is at least 60%, 85%, 90%, 95%, 98% or 99% identical to amino acid residues 15-50 of SEQ ID NO: 1. In some embodiments, a truncated protein degron variant comprises an amino acid sequence that is at least 60%, 85%, 90%, 95%, 98% or 99% identical to amino acid residues 15-50 of SEQ ID NO: 1 and comprises one or more amino acid substitutions at one or more positions selected from R15, P16, Q18, E20, K37, P44, C47, and C50 relative to SEQ ID NO: 1.

Some aspects of the disclosure provide truncated protein degron variants having between 1 and 10 amino acid substitutions (e.g., mutations) relative to SEQ ID NO: 1 (e.g., 1, 2, 3, 4, 5, 6, 7, 8, etc.). Some aspects of the disclosure provide truncated protein degron variants having more than 10 amino acid substitutions (e.g., mutations) relative to SEQ ID NO: 1 (e.g., 15, 20, 25, 30, 40, etc.). In some embodiments, a truncated protein degron variant has 2, 3, 4, 5, 6, 7, 8, 9, or 10 amino acid substitutions relative to a SEQ ID NO: 1. The mutations disclosed herein are not exclusive of other mutations which may occur or be introduced. For example, a truncated protein degron variant may have a mutation as described herein in addition to at least one mutation not described herein (e.g., 1, 2, 3, 4, 5, etc. additional mutations).

In some embodiments, a truncated protein degron variant comprises one or more amino acid substitutions at a position selected from R15, P16, Q18, E20, K37, P44, C47, and C50 relative to SEQ ID NO: 1. In some embodiments, a truncated protein degron variant comprises one or more amino acid substitutions selected from R15L, P16L, Q18F, E20P, K37N, P44L, C47Y, and C50Y relative to SEQ ID NO: 1. In some embodiments, a truncated protein degron variant comprises the following amino acid substitutions relative to SEQ ID NO: 1: R15L, P16L, Q18F, E20P, K37N, P44L, C47Y, and C50Y.

In some embodiments, a truncated protein degron variant comprises the following amino acid substitutions relative to SEQ ID NO: 1: R15L, P16L, Q18F, E20P, K37N, P44L, C47Y, and C50Y. In some embodiments, a truncated protein degron variant comprises the following amino acid substitutions relative to SEQ ID NO: 1: R15L, P16L, Q18F, E20P, K37N, P44L, C47Y, and C50Y. In some embodiments, a truncated protein degron variant comprises the following amino acid substitutions relative to SEQ ID NO: 1: R15L, P16L, Q18F, E20P, K37N, P44L, C47Y, and C50Y. In some embodiments, a truncated protein degron variant comprises the following amino acid substitutions relative to SEQ ID NO: 1: R15L, P16L, Q18F, E20P, K37N, P44L, C47Y, and C50Y. In some embodiments, a truncated protein degron variant comprises the following amino acid substitutions relative to SEQ ID NO: 1: R15L, P16L, Q18F, E20P, K37N, P44L, and C47Y. In some embodiments, a truncated protein degron variant comprises the following amino acid substitutions relative to SEQ ID NO: 1: R15L, P16L, Q18F, E20P, K37N, and P44L. In some embodiments, a truncated protein degron variant comprises the following amino acid substitutions relative to SEQ ID NO: 1: R15L, P16L, Q18F, E20P, and K37N. In some embodiments, a truncated protein degron variant comprises the following amino acid substitutions relative to SEQ ID NO: 1: P16L, Q18F, E20P, and K37N.

In some embodiments, a truncated protein degron variant lacks one or more amino acids (e.g., comprises a deletion or truncation) at one or more of the following ranges of positions: 1-14, 1-15, 40-60, 45-60, 48-60, 51-60, and 53-60. In some embodiments, a truncated protein degron variant lacks amino acids at positions 1-14 relative to SEQ ID NO: 1. In some embodiments, a truncated protein degron variant lacks amino acids at positions 51-60 relative to SEQ ID NO: 1. In some embodiments, a truncated protein degron variant lacks amino acids at positions 1-14 and 53-60 relative to SEQ ID NO: 1. In some embodiments, a truncated protein degron variant lacks amino acids at positions 1-14 and 51-60 relative to SEQ ID NO: 1. In some embodiments, a truncated protein degron variant lacks amino acids at positions 1-14 and 48-60 relative to SEQ ID NO: 1. In some embodiments, a truncated protein degron variant lacks amino acids at positions 1-14 and 45-60 relative to SEQ ID NO: 1. In some embodiments, a truncated protein degron variant lacks amino acids at positions 1-14 and 40-60 relative to SEQ ID NO: 1. In some embodiments, a truncated protein degron variant lacks amino acids at positions 1-15 and 40-60 relative to SEQ ID NO: 1.

In some embodiments, a truncated protein degron variant lacks amino acids at positions 1-14 relative to SEQ ID NO: 1 and comprises the following amino acid substitutions relative to SEQ ID NO: 1: R15L, P16L, Q18F, E20P, K37N, P44L, C47Y, and C50Y.

In some embodiments, a truncated protein degron variant lacks amino acids at positions 51-60 relative to SEQ ID NO: 1 and comprises the following amino acid substitutions relative to SEQ ID NO: 1: R15L, P16L, Q18F, E20P, K37N, P44L, C47Y, and C50Y.

In some embodiments, a truncated protein degron variant lacks amino acids at positions 1-14 and 53-60 relative to SEQ ID NO: 1 and comprises the following amino acid substitutions relative to SEQ ID NO: 1: R15L, P16L, Q18F, E20P, K37N, P44L, C47Y, and C50Y.

In some embodiments, a truncated protein degron variant lacks amino acids at positions 1-14 and 51-60 relative to SEQ ID NO: 1 and comprises the following amino acid substitutions relative to SEQ ID NO: 1: R15L, P16L, Q18F, E20P, K37N, P44L, C47Y, and C50Y.

In some embodiments, a truncated protein degron variant lacks amino acids at positions 1-14 and 48-60 relative to SEQ ID NO: 1 and comprises the following amino acid substitutions relative to SEQ ID NO: 1: R15L, P16L, Q18F, E20P, K37N, P44L, and C47Y.

In some embodiments, a truncated protein degron variant lacks amino acids at positions 1-14 and 45-60 relative to SEQ ID NO: 1 and comprises the following amino acid substitutions relative to SEQ ID NO: 1: R15L, P16L, Q18F, E20P, K37N, and P44L.

In some embodiments, a truncated protein degron variant lacks amino acids at positions 1-14 and 40-60 relative to SEQ ID NO: 1 and comprises the following amino acid substitutions relative to SEQ ID NO: 1: R15L, P16L, Q18F, E20P, and K37N.

In some embodiments, a truncated protein degron variant lacks amino acids at positions 1-15 and 40-60 relative to SEQ ID NO: 1 and comprises the following amino acid substitutions relative to SEQ ID NO: 1: P16L, Q18F, E20P, and K37N.

In some embodiments, a protein degron variant has at least 70% sequence identity to (e.g., at least 70%, at least 80%, at least 85%, at least 90%, at least 95%, at least 96%, at least 97%, at least 98%, at least 99%, at least 99.5%, at least 99.9%, or more identity to a sequence selected from SEQ ID NOs.: 46-53. In some embodiments, a protein degron variant comprises or consists of an amino acid sequence set forth in any one of SEQ ID NOs.: 46-53. In some embodiments, a protein degron variant comprises or consists of an amino acid sequence set forth in SEQ ID NO: 49.

In some embodiments, the protein degron is optimized for degradation by a small molecule. In some embodiments, the small molecule is not thalidomide, lenalidomide, pomalidomide, avadomide, or iberdomide. In some embodiments, the small molecule is VS-777, PT-179, or PK-1016. In some embodiments, the small molecule is PT-179.

### Methods of Protein Degradation

Some aspects of this disclosure provide methods for using a protein degron variant provided herein. In some embodiments, such methods include degrading a target polypeptide in a cell, for example, ex vivo, in vitro, or in vivo (e.g., in a subject), with a protein degron variant provided herein.

Protein degradation may occur by contacting a cell or subject containing a target protein (e.g., a protein that is desired to be degraded, and contains a protein degron tag described herein) with one or more small molecule CRBN substrates. A CRBN substrate may be a small molecule that binds to CRBN, and induces a structural change in CRBN that results in CRBN having the ability to bind to one or more neosubstrates. In some embodiments, the one or more neosubstrates comprises a protein degron tag as described by the disclosure. In some embodiments, the one or more neosubstrates comprise a fusion protein. In some embodiments, the fusion protein comprises a protein degron described herein and the target protein. In some embodiments, a CRBN substrate comprises VS-777, PT-179, or PK-1016. In some embodiments, a CRBN substrate comprises PT-179.

As described in the Example, administration of certain CRBN substrates (e.g., PT-179) to mammalian cells results in fewer differentially expressed genes (DEGs) in the cells relative to other canonical CRBN substrates, such as thalidomide or pomalidomide. Without wishing to be bound by any particular theory, the reduction of DEGs mediated by non-canonical CRBNs is indicative of less off-target protein degradation mediated by inducer-CRBN ternary complexes. In some embodiments, the method mitigates off-target interactions compared to an immunomodulatory drug. In some embodiments, the immunomodulatory drug (IMiD) is selected from the group consisting of thalidomide, lenalidomide, pomalidomide, avadomide, and iberdomide. In some embodiments, the IMiD is the immunomodulatory drug is pomalidomide. In some embodiments, a protein degron described herein does not bind to CRBN in the presence of thalidomide, lenalidomide, pomalidomide, avadomide, or iberdomide.

In some embodiments, a cell is a mammalian cell, for example, a human cell, dog cell, cat cell, horse cell, pig cell, rodent (e.g., mouse, rat, hamster, etc.) cell, or a non-human primate (e.g., monkey) cell. In some embodiments, a cell is in a subject, for example, a human subject, dog subject, cat subject, horse subject, pig subject, rodent subject, or non-human primate subject. In some embodiments, the subject is a human subject. The cell or human subject typically expresses cereblon (CRBN).

Upon contact with the small molecule CRBN substrate, a ternary complex forms between the CRBN, a small molecule CRBN substrate, and a neosubstrate (e.g., a protein degron tag described herein). In some embodiments the small molecule CRBN substrate comprises VS-777, PT-179, or PK-1016. In some embodiments the small molecule CRBN substrate is VS-777, PT-179, or PK-1016. In some embodiments the small molecule CRBN substrate comprises PT-179. In some embodiments the small molecule CRBN substrate is PT-179. In some aspects, provided herein is a complex comprising a CRBN protein simultaneously bound to a small molecule CRBN substrate (e.g., PT-179) and a protein degron described herein. Upon formation of the complex, the CRBN (e.g., via an E3 ubiquitin ligase complex) mediates transfer of one or more ubiquitin proteins to the protein containing the protein degron tag, thus marking the protein for degradation. A target protein may be an endogenous protein (e.g., a protein that is naturally expressed by the cell or subject) or a protein that is heterologous to the subject (e.g., a recombinantly expressed protein). In some embodiments, an endogenous target protein is modified (e.g., modified to contain a protein degron as described herein). In some embodiments, a heterologous protein (e.g., a recombinant protein) is engineered to comprise one or more protein degron tags described herein. For example, a recombinant protein may be engineered to be expressed as a fusion protein comprising the protein of interest and a protein degron described herein. In some embodiments, a protein of interest (e.g., a recombinant protein) is a therapeutic protein. Examples of therapeutic proteins include, but are not limited to, antibodies, antibody fragments (e.g., single chain antibodies, etc.), therapeutic peptides (e.g., gene replacement therapies), toxins, chimeric antigen receptor (CAR) components, etc. In some embodiments, inclusion of a protein degron described herein on a recombinant protein (e.g., therapeutic protein) is advantageous because it provides an “off” switch by which the level or activity of the recombinant protein may be regulated (e.g., reduced by protein degradation) using a small molecule CRBN substrate.

### Nucleic Acids, Expression Vectors, and Fusion Proteins

In some aspects, the disclosure relates to a nucleic acid encoding an evolved protein degron described herein. In some embodiments, the nucleic acid has at least 50% sequence identity to (e.g., at least 60%, at least 70%, at least 80%, at least 85%, at least 90%, at least 95%, at least 96%, at least 97%, at least 98%, at least 99%, at least 99.5%, at least 99.9%, or more identity to a nucleic acid sequence selected from SEQ ID NOs.: 59-95. In some embodiments, the nucleic acid has at least 50% sequence identity to (e.g., at least 60%, at least 70%, at least 80%, at least 85%, at least 90%, at least 95%, at least 96%, at least 97%, at least 98%, at least 99%, at least 99.5%, at least 99.9%, or more identity to a nucleic acid sequence selected from SEQ ID NOs.: 128-129. In some embodiments, the nucleic acid has at least 70% sequence identity to (e.g., at least 70%, at least 80%, at least 85%, at least 90%, at least 95%, at least 96%, at least 97%, at least 98%, at least 99% identity) to a nucleic acid sequence selected from SEQ ID NOs.: 59-95. In some embodiments, the nucleic acid has at least 70% sequence identity to (e.g., at least 70%, at least 80%, at least 85%, at least 90%, at least 95%, at least 96%, at least 97%, at least 98%, at least 99% identity) to a nucleic acid sequence selected from SEQ ID NOs.: 128-129. In some embodiments, the nucleic acid comprises the sequence set forth in any one of SEQ ID NOs: 59-95. In some embodiments, the nucleic acid comprises the sequence set forth in any one of SEQ ID NOs: 128-129. In some embodiments, the nucleic acid sequence is codon-optimized. In some embodiments, the nucleic acid sequence is codon-optimized for enhanced expression in desired cells. In some embodiments, the nucleic acid sequence is codon-optimized for expression in mammalian cells. In some embodiments, the nucleic acid has at least 50% sequence identity to (e.g., at least 60%, at least 70%, at least 80%, at least 85%, at least 90%, at least 95%, at least 96%, at least 97%, at least 98%, at least 99%, at least 99.5%, at least 99.9%, or more identity) to a nucleic acid sequence selected from SEQ ID NOs.: 96-123, 126, and 127. In some embodiments, the nucleic acid has at least 70% sequence identity to (e.g., at least 70%, at least 80%, at least 85%, at least 90%, at least 95%, at least 96%, at least 97%, at least 98%, at least 99% identity) to a nucleic acid sequence selected from SEQ ID NOs.: 96-123, 126, and 127. In some embodiments, the nucleic acid comprises the sequence set forth in any one of SEQ ID NOs.: 96-123, 126, and 127.

In some aspects, provided herein is an expression vector comprising a nucleic acid encoding the evolved protein degron disclosed herein. In some embodiments, the vector is a phage, plasmid, cosmid, bacmid, or viral vector. In some embodiments, the nucleic acid comprises the sequence set forth in any one of SEQ ID NOs: 59-95. In some embodiments, the nucleic acid comprises the sequence set forth in any one of SEQ ID NOs: 128-129. In some embodiments, the nucleic acid sequence is codon-optimized. In some embodiments, the nucleic acid sequence is codon-optimized for enhanced expression in desired cells. In some embodiments, the nucleic acid sequence is codon-optimized for expression in mammalian cells. In some embodiments, the nucleic acid comprises the sequence set forth in any one of SEQ ID NOs.: 96-123. In some embodiments, the nucleic acid comprises the sequence set forth in any one of SEQ ID NOs.: 126-127. In some embodiments, the nucleic acid sequence is codon-optimized for expression in human cells.

In some aspects, provided herein is a host cell comprising the evolved protein degron disclosed herein or the expression vector disclosed herein. In some embodiments, the host cell is a bacterial cell or mammalian cell. In some embodiments, the host cell is a bacterial cell. In some embodiments, the host cell is a mammalian cell. In some embodiments, a mammalian cell is a human cell. In some embodiments, a mammalian cell is a non-human primate cell, dog cell, cat cell, horse cell, guinea pig cell, hamster cell pig cell, or mouse cell. In some embodiments, the host cell is an E. coli cell.

In some aspects, provided herein is a fusion protein comprising the evolved protein degron as described herein and a target protein. In some embodiments, the target protein is an endogenous protein. In some embodiments, the target protein is a recombinant protein. In some embodiments, the target protein is a therapeutic protein. In some embodiments, the therapeutic protein is selected from the group consisting of antibodies, antibody fragments (e.g., single chain antibodies, etc.), therapeutic peptides (e.g., gene replacement therapies), toxins, or chimeric antigen receptor (CAR) components.

### Methods of Use

Some aspects of the disclosure relate to methods of degrading a target protein in a cell. In some embodiments, the method comprises contacting a cell comprising cereblon (CRBN), and a target protein having the protein degron described herein, with a small molecule CRBN substrate.

The target protein may be an endogenous protein (e.g., a protein that is naturally expressed by the cell or subject) or a protein that is heterologous to the subject (e.g., a recombinantly expressed protein). In some embodiments, an endogenous target protein is modified (e.g., modified to contain a protein degron as described herein). In some embodiments, a heterologous protein (e.g., a recombinant protein) is engineered to comprise one or more protein degron tags described herein. For example, a recombinant protein may be engineered to be expressed as a fusion protein comprising the protein of interest and a protein degron described herein. In some embodiments, a protein of interest (e.g., a recombinant protein) is a therapeutic protein. Examples of therapeutic proteins include, but are not limited to, antibodies, antibody fragments (e.g., single chain antibodies, etc.), therapeutic peptides (e.g., gene replacement therapies), toxins, chimeric antigen receptor (CAR) components.

The cell may be a prokaryotic cell or a eukaryotic cell. In some embodiments, the cell is a eukaryotic cell, for example, a human cell, a mouse cell, a dog cell, a cat cell, a horse cell, a guinea pig cell, a pig cell, a hamster cell, a non-human primate (e.g. monkey) cell, etc. In some embodiments, the cell is a human cell. In some embodiments, the cell is in a subject (e.g., the cell is in vivo). In some embodiments, the subject is a non-mammalian subject. In some embodiments, the subject is a mammalian subject. In some embodiments, the subject is a dog, cat, horse, pig, rodent, human, or non-human primate. In some embodiments, the subject is a human. Examples of therapeutic proteins include, but are not limited to, antibodies, antibody fragments (e.g., single chain antibodies, etc.), therapeutic peptides (e.g., gene replacement therapies), toxins, chimeric antigen receptor (CAR) components.

In some embodiments, the small molecule CRBN substrate is not thalidomide, lenalidomide, pomalidomide, avadomide, or iberdomide. In some embodiments, the small molecule CRBN substrate comprises VS-777, PT-179, or PK-1016. In some embodiments, the small molecule CRBN substrate is PT-179.

### Engineering of Protein Degron Variants Using PACE and PANCE

Some aspects of the disclosure relate to methods for evolving a protein degron, the methods comprising contacting a population of bacterial host cells with a population of phages comprising a first nucleic acid encoding a first fusion protein, and deficient in a full-length pIII gene, wherein the first fusion protein comprises a protein degron of interest and an RNA polymerase subunit. In some embodiments, the population of phages allows for expression of the first fusion protein in the host cells. In some embodiments, the host cells are suitable for phage infection, replication, and packaging. In some embodiments the host cells comprise a second nucleic acid encoding full-length pIII protein, and a third nucleic acid sequence encoding a second fusion protein. In some embodiments, the second fusion protein comprises a cereblon (CRBN) and a repressor element. In some embodiments, the protein degron of interest comprises the amino acid sequence set forth in SEQ ID NO: 1. In some embodiments, the expression of the pIII gene is dependent on interaction of the protein degron of interest of the first fusion protein with the CRBN of the second fusion protein. In some embodiments, the methods further comprise incubating the population of host cells and M13 phages under conditions allowing for the modification of the third nucleic acid, the production of infectious M13 phage, and the infection of host cells with M13 phage. In some embodiments, the conditions allowing for the modification of the third nucleic acid, are the presence of a small molecule. In some embodiments, the infected cells are removed from the population of host cells. In some embodiments, the population of host cells is replenished with fresh host cells that are not infected by M13 phage. In some embodiments, the methods further comprise isolating a modified M13 phage replication product encoding an evolved variant of the first fusion protein from the population of host cells. In some embodiments, the RNA polymerase subunit is an RNA polymerase omega (RpoZ) or RNA polymerase alpha (RpoA) subunit. In some embodiments, the promoter is a pLac-derived promoter. In some embodiments, the promoter is a lacZ promoter or a mutant lacZ promoter (e.g., PlacZ-opt). In some embodiments, the RNA polymerase subunit is RNA polymerase omega (RpoZ) or RNA polymerase alpha (RpoA) subunit, and the promoter is a lacZ promoter or a mutant lacZ promoter (e.g., PlacZ-opt). In some embodiments, the repressor element is a phage repressor. Non-limiting examples of phage repressors include lambda, 434, and P22 phage repressors. Phage repressors are known to those in the art (see e.g., M. Ptashne et al. Autoregulation and Function of a Repressor in Bacteriophage Lambda.Science 194,156-161 (1976).DOI: 10.1126/science.959843; Chen J, Pongor S, Simoncsits A. Recognition of DNA by single-chain derivatives of the phage 434 repressor: high affinity binding depends on both the contacted and non-contacted base pairs. Nucleic Acids Res. 1997 Jun. 1; 25 (11): 2047-54. doi: 10.1093/nar/25.11.2047. PMID: 9153301; PMCID: PMC146726; and Sauer R T, Nelson H C, Hehir K, Hecht M H, Gimble F S, DeAnda J, Poteete A R. The lambda and P22 phage repressors. J Biomol Struct Dyn. 1983 December; 1 (4): 1011-22. doi: 10.1080/07391102.1983.10507499. PMID: 6242868, the entire contents of each of which are incorporated by reference).

An exemplary repressor is the lambda repressor protein (c1) that efficiently represses the lambda promoter pR and can be modified to include a desired protease cleavage site (see, e.g., Sices, H. J.; Kristie, T. M., A genetic screen for the isolation and characterization of site-specific proteases. Proc Natl Acad Sci USA 1998, 95 (6), 2828-33; and Sices, H. J.; Leusink, M. D.; Pacheco, A.; Kristie, T. M., Rapid genetic selection of inhibitor-resistant protease mutants: clinically relevant and novel mutants of the HIV protease. AIDS Res Hum Retroviruses 2001, 17 (13), 1249-55, the entire contents of each of which are incorporated herein by reference). In some embodiments, the repressor is a variant of a phage repressor (e.g., a single-chain variant). In some embodiments, a phage repressor comprises a single-chain phage repressor. In some embodiments, a phage repressor comprises a single-chain variant of a 434 phage repressor (e.g., an RR69 phage repressor). In some embodiments, a phage repressor comprises a single-chain variant of a p22 phage repressor (e.g., sc-p22cI repressor).

In some embodiments, the evolved protein degron comprises a sequence that is at least 50% identical (e.g., at least 50%, at least 60%, at least 70%, at least 80%, at least 85%, at least 90%, at least 95%, at least 96%, at least 97%, at least 98%, or at least 99% identical) to a sequence selected from SEQ ID NOs.: 2-58. In some embodiments, the evolved protein degron comprises a sequence that is at least 50% identical (e.g., at least 50%, at least 60%, at least 70%, at least 80%, at least 85%, at least 90%, at least 95%, at least 96%, at least 97%, at least 98%, or at least 99% identical) to a sequence set forth in SEQ ID NO.: 124. In some embodiments, the evolved protein degron comprises a sequence that is at least 50% identical (e.g., at least 50%, at least 60%, at least 70%, at least 80%, at least 85%, at least 90%, at least 95%, at least 96%, at least 97%, at least 98%, or at least 99% identical) to a sequence set forth in SEQ ID NO.: 125. In some embodiments, the evolved protein degron comprises the sequence set forth in any one of SEQ ID NOs.: 2-58. In some embodiments, the evolved protein degron comprises the sequence set forth in SEQ ID NOs.: 124 or 125. In some embodiments, the evolved protein degron comprises the sequence set forth in SEQ ID NO: 37. In some embodiments, the evolved protein degron comprises the sequence set forth in SEQ ID NO: 49. In some embodiments, the evolved protein degron comprises the sequence set forth in SEQ ID NO: 125.

In some embodiments, the incubating of the host cells is for a time sufficient for at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles. In certain embodiments, the viral vector is an M13 phage, and the length of a single viral life cycle is about 10-20 minutes.

In some embodiments, the cells are contacted and/or incubated in suspension culture. For example, in some embodiments, bacterial cells are incubated in suspension culture in liquid culture media. Suitable culture media for bacterial suspension culture will be apparent to those of skill in the art, and the invention is not limited in this regard. See, for example, Molecular Cloning: A Laboratory Manual, 2nd Ed., ed. by Sambrook, Fritsch, and Maniatis (Cold Spring Harbor Laboratory Press: 1989); Elizabeth Kutter and Alexander Sulakvelidze: Bacteriophages: Biology and Applications. CRC Press; 1st edition (December 2004), ISBN: 0849313368; Martha R. J. Clokie and Andrew M. Kropinski: Bacteriophages: Methods and Protocols, Volume 1: Isolation, Characterization, and Interactions (Methods in Molecular Biology) Humana Press; 1st edition (December 2008), ISBN: 1588296822; Martha R. J. Clokie and Andrew M. Kropinski: Bacteriophages: Methods and Protocols, Volume 2: Molecular and Applied Aspects (Methods in Molecular Biology) Humana Press; 1st edition (December 2008), ISBN: 1603275649; all of which are incorporated herein in their entirety by reference for disclosure of suitable culture media for bacterial host cell culture). Suspension culture typically requires the culture media to be agitated, either continuously or intermittently. This is achieved, in some embodiments, by agitating or stirring the vessel comprising the host cell population. In some embodiments, the outflow of host cells and the inflow of fresh host cells is sufficient to maintain the host cells in suspension. This in particular, if the flow rate of cells into and/or out of the culture vessel is high.

Generally, an accessory plasmid is required for selection of viral vectors, for example, the accessory plasmid comprising the gene required for the generation of infectious phage particles that is lacking from the phages being evolved. In some embodiments, an accessory plasmid comprises a fusion protein comprising cereblon and a repressor element. In some embodiments, the host cells are generated by contacting an uninfected host cell with the relevant vectors, for example, the accessory plasmid and, optionally, a mutagenesis plasmid, and growing an amount of host cells sufficient for the replenishment of the host cell population in a continuous evolution experiment. Methods for the introduction of plasmids and other gene constructs into host cells are well known to those of skill in the art and the invention is not limited in this respect. For bacterial host cells, such methods include, but are not limited to electroporation and heat-shock of competent cells. In some embodiments, the accessory plasmid comprises a selection marker, for example, an antibiotic resistance marker, and the fresh host cells are grown in the presence of the respective antibiotic to ensure the presence of the plasmid in the host cells. Where multiple plasmids are present, different markers are typically used. Such selection markers and their use in cell culture are known to those of skill in the art, and the invention is not limited in this respect.

In some embodiments, the host cell population in a continuous evolution experiment is replenished with fresh host cells growing in a parallel, continuous culture. In some embodiments, the cell density of the host cells in the host cell population contacted with the viral vector and the density of the fresh host cell population is substantially the same.

In some embodiments, the host cell population is contacted with a mutagen. In some embodiments, the cell population contacted with the viral vector (e.g., the phage) is continuously exposed to the mutagen at a concentration that allows for an increased mutation rate of the gene of interest, but is not significantly toxic for the host cells during their exposure to the mutagen while in the host cell population. In other embodiments, the host cell population is contacted with the mutagen intermittently, creating phases of increased mutagenesis, and accordingly, of increased viral vector diversification. For example, in some embodiments, the host cells are exposed to a concentration of mutagen sufficient to generate an increased rate of mutagenesis in the gene of interest for about 10%, about 20%, about 50%, or about 75% of the time.

In some embodiments, the host cells comprise a mutagenesis expression construct, for example, in the case of bacterial host cells, a mutagenesis plasmid. In some embodiments, the mutagenesis plasmid comprises a gene expression cassette encoding a mutagenesis-promoting gene product, for example, a proofreading-impaired DNA polymerase. In other embodiments, the mutagenesis plasmid, including a gene involved in the SOS stress response, (e.g., UmuC, UmuD′, and/or RecA). In some embodiments, the mutagenesis-promoting gene is under the control of an inducible promoter. Suitable inducible promoters are well known to those of skill in the art and include, for example, arabinose-inducible promoters, tetracycline or doxycyclineinducible promoters, and tamoxifen-inducible promoters. In some embodiments, the host cell population is contacted with an inducer of the inducible promoter in an amount sufficient to effect an increased rate of mutagenesis. For example, in some embodiments, a bacterial host cell population is provided in which the host cells comprise a mutagenesis plasmid in which a dnaQ926, UmuC, UmuD′, and RecA expression cassette is controlled by an arabinose-inducible promoter. In some such embodiments, the population of host cells is contacted with the inducer, for example, arabinose in an amount sufficient to induce an increased rate of mutation.

The use of an inducible mutagenesis plasmid allows one to generate a population of fresh, uninfected host cells in the absence of the inducer, thus avoiding an increased rate of mutation in the fresh host cells before they are introduced into the population of cells contacted with the viral vector. Once introduced into this population, however, these cells can then be induced to support an increased rate of mutation, which is particularly useful in some embodiments of continuous evolution. For example, in some embodiments, the host cell comprise a mutagenesis plasmid as described herein, comprising an arabinose-inducible promoter driving expression of dnaQ926, UmuC, UmuD′, and RecA730 from a pBAD promoter (see, e.g., Khlebnikov A, Skaug T, Keasling J D. Modulation of gene expression from the arabinose-inducible araBAD promoter. J Ind Microbiol Biotechnol. 2002 July; 29 (1): 34-7; incorporated herein by reference for disclosure of a pBAD promoter). In some embodiments, the mutagenesis plasmid is an MP4 mutagenesis plasmid or an MP6 mutagenesis plasmid. The MP4 and MP6 mutagenesis plasmids are described, for example in PCT Application PCT/US2016/27795, published as WO 2016/168631 on Oct. 20, 2016, the content of which is incorporated herein in its entirety. The MP4 mutagenesis plasmid comprises the following genes: dnaQ926, dam, seqA¹⁷. The MP6 mutagenesis plasmid comprises the following genes: dnaQ926, dam, seqA, emrR, Ugi, and CDA1²².

In some embodiments, the fresh host cells are not exposed to arabinose, which activates expression of the above identified genes and, thus, increases the rate of mutations in the arabinose-exposed cells, until the host cells reach the lagoon in which the population of selection phage replicates. Accordingly, in some embodiments, the mutation rate in the host cells is normal until they become part of the host cell population in the lagoon, where they are exposed to the inducer (e.g., arabinose) and, thus, to increased mutagenesis. In some embodiments, a method of continuous evolution is provided that includes a phase of diversifying the population of viral vectors by mutagenesis, in which the cells are incubated under conditions suitable for mutagenesis of the viral vector in the absence of stringent selection for the mutated replication product of the viral vector encoding the evolved protein. This is particularly useful in embodiments in which a desired function to be evolved is not merely an increase in an already present function, for example, an increase in the transcriptional activation rate of a transcription factor, but the acquisition of a function not present in the gene of interest at the outset of the evolution procedure (for example, altered ligand binding specificity). A step of diversifying the pool of mutated versions of the gene of interest within the population of viral vectors, for example, of phage, allows for an increase in the chance to find a mutation that conveys the desired function.

In addition to altering the rate of mutagenesis, the selective stringency of host cells can be tuned. Such methods involving host cells of varying selective stringency allow for harnessing the power of continuous evolution methods as provided herein for the evolution of functions that are completely absent in the initial version of the gene of interest, for example, for the evolution of a transcription factor recognizing a foreign target sequence that a native transcription factor, used as the initial gene of interest, does not recognize at all. Or, for another example, the recognition of a desired target sequence by a DNA-binding protein, a recombinase, a nuclease, a zinc finger protein, or an RNA-polymerase, that does not bind to or does not exhibit any activity directed towards the desired target sequence.

Other selection schemes for gene products having a desired activity are well known to those of skill in the art or will be apparent from the instant disclosure. Selection strategies that can be used in continuous evolution processes and methods as provided herein include, but are not limited to, selection strategies useful in two-hybrid screens. For example, the variant protein degron selection strategy described in more detail elsewhere herein is an example of a receptor recognition selection strategy.

### Vectors and Reagents

Some aspects of this disclosure provide vectors and reagents for carrying out the inventive continuous protein degron evolution processes.

In some embodiments, a selection phage is provided that comprises a phage genome deficient in at least one gene required for the generation of infectious phage particles and a gene encoding a degron of interest to be evolved (e.g. a super degron comprising the amino acid sequence set forth in SEQ ID NO: 1).

For example, in some embodiments, a selection phage as described in in PCT Application PCT/US2009/056194, published as WO 2010/028347 on Mar. 11, 2010; PCT Application PCT/US2011/066747, published as WO 2012/088381 on Jun. 28, 2012; and U.S. Non-provisional Application, U.S. Ser. No. 13/922,812, filed on Jun. 20, 2013, the entire contents of each of which are incorporated herein by reference, is provided, that comprises a multiple cloning site for insertion of a nucleic acid sequence encoding a degron of interest.

Such selection phage vectors typically comprise an M13 phage genome deficient in a gene required for the generation of infectious M13 phage particles, for example, a full-length gIII. In some embodiments, the selection phage comprises a phage genome providing all other phage functions required for the phage life cycle except the gene required for generation of infectious phage particles. In some such embodiments, an M13 selection phage is provided that comprises a gI, gII, gIV, gV, gVI, gVII, gVIII, gIX, and a gX gene, but not a full-length gIII. In some embodiments, the selection phage comprises a 3′-fragment of gIII, but no full-length gIII. The 3′-end of gIII comprises a promoter and retaining this promoter activity is beneficial, in some embodiments, for an increased expression of gVI, which is immediately downstream of the gIII 3′-promoter, or a more balanced (wild-type phage-like) ratio of expression levels of the phage genes in the host cell, which, in turn, can lead to more efficient phage production. In some embodiments, the 3′-fragment of gIII gene comprises the 3′-gIII promoter sequence. In some embodiments, the 3′-fragment of gIII comprises the last 180 bp, the last 150 bp, the last 125 bp, the last 100 bp, the last 50 bp, or the last 25 bp of gIII. In some embodiments, the 3′-fragment of gIII comprises the last 180 bp of gIII. In some embodiments, the multiple cloning site for insertion of the gene encoding the protein degron of interest is located downstream of the gVIII 3′-terminator and upstream of the gIII-3′-promoter.

Some aspects of this invention provide a vector system for continuous evolution procedures, comprising of a viral vector, for example, a selection phage and a matching accessory plasmid. In some embodiments, a vector system for phage-based continuous directed evolution is provided that comprises (a) a selection phage comprising a gene encoding the protein to be evolved (e.g. protein degron), wherein the phage genome is deficient in a gene required to generate infectious phage; and (b) an accessory plasmid comprising the gene required to generate infectious phage particle under the control of a conditional promoter. In some embodiments, the accessory plasmid comprises a nucleic acid encoding a fusion protein comprising cereblon and a repressor element. In some embodiments, the conditional promoter is activated by the interaction of the protein to be evolved, encoded on the selection phage and the protein encoded on the accessory plasmid.

In some embodiments, the selection phage is an M13 phage as described herein. For example, in some embodiments, the selection phage comprises an M13 genome including all genes required for the generation of phage particles, for example, gI, gII, gIV, gV, gVI, gVII, gVIII, gIX, and gX gene, but not a full-length gIII gene. In some embodiments, the selection phage genome comprises an F1 or an M13 origin of replication. In some embodiments, the selection phage genome comprises a 3′-fragment of gIII gene. In some embodiments, the selection phage comprises a multiple cloning site upstream of the gIII 3′-promoter and downstream of the gVIII 3′-terminator for insertion of a gene encoding a degron of interest.

The vector system may further comprise a helper phage, wherein the selection phage does not comprise all genes for the generation of infectious phage particles, and wherein the helper phage complements the genome of the selection phage, so that the helper phage genome and the selection phage genome together comprise at least one functional copy of all genes for the generation of phage particles, but are deficient in at least one gene required for the generation of infectious phage particles, which is provided by an accessory plasmid.

In some embodiments, the accessory plasmid of the vector system comprises an expression cassette comprising the gene required for the generation of infectious phage under the control of a conditional promoter. In some embodiments, the accessory plasmid of the vector system comprises a gene encoding pIII under the control of a conditional promoter. In some embodiments, the accessory plasmid comprises a nucleic acid encoding a fusion protein comprising cereblon and a repressor element. In some embodiments, the activity of the conditional promoter is dependent on interaction of the protein to be evolved, encoded on the selection phage and the protein encoded on the accessory plasmid. In some embodiments, the protein to be evolved is expressed by the host cells. In some embodiments, the protein to be evolved is a super degron (e.g., SD0, SEQ ID NO:1). In some embodiments, the protein to be evolved is fused to a RNA polymerase that drives expression of the gene encoding pIII by interacting with the conditional promoter. In some embodiments, the RNA polymerase is RNA polymerase omega (RpoZ) or RNA polymerase alpha (RpoA), and the conditional promoter is a lacZ promoter or a mutant lacZ promoter (e.g., PlacZ-opt).

In some embodiments, the vector system further comprises a mutagenesis plasmid, for example, an arabinose-inducible mutagenesis plasmid as described herein (e.g., MP4 or MP6).

In some embodiments, the vector system further comprises a helper plasmid providing expression constructs of any phage gene not comprised in the phage genome of the selection phage or in the accessory plasmid.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

A phage-assisted continuous evolution (PACE) was used to reprogram molecular glue complexes and applied the system to evolve a small (36-residue) zinc finger domain that engages IMiD-bound human CRBN. This small degron was evolved in the presence of IMiD analogs bearing phthalimide-ring substitutions that disrupt binding to native protein neosubstrates and confirmed that these ‘bumped’ phthalimide analogs exhibit greatly reduced off-target activity in human cells compared to the canonical IMiD pomalidomide. Human proteins tagged with the evolved degron are rapidly and potently degraded by an otherwise-inert IMiD analog, with no hook effect. The evolved degron is small enough to be efficiently inserted into a targeted site in the human genome using prime editing for in-frame, endogenous gene tagging. Zinc finger variants that engage mouse CRBN were also evolved, enabling IMiD-analog-induced protein degradation in mouse cells. These findings collectively establish a continuous evolution platform for the rapid remodeling of molecular glue complexes and provide degrons that recognize new, highly specific molecular glues.

### Example 1

This example describes evolution of a super degron to form a strong ternary complex with small molecule-bound cereblon (e.g., PT-179 bound cereblon) and to serve as a potent, small molecule responsive degron tag. The super degron is a 60 amino acid zinc finger-derived protein (SEQ ID NO: 1), previously reported by Jan et al., Sci. Transl. Med. 2021, 13 (575), eabb6295, that forms a ternary complex with cereblon and immunomodulatory drugs (IMiDs), such as pomalidomide.

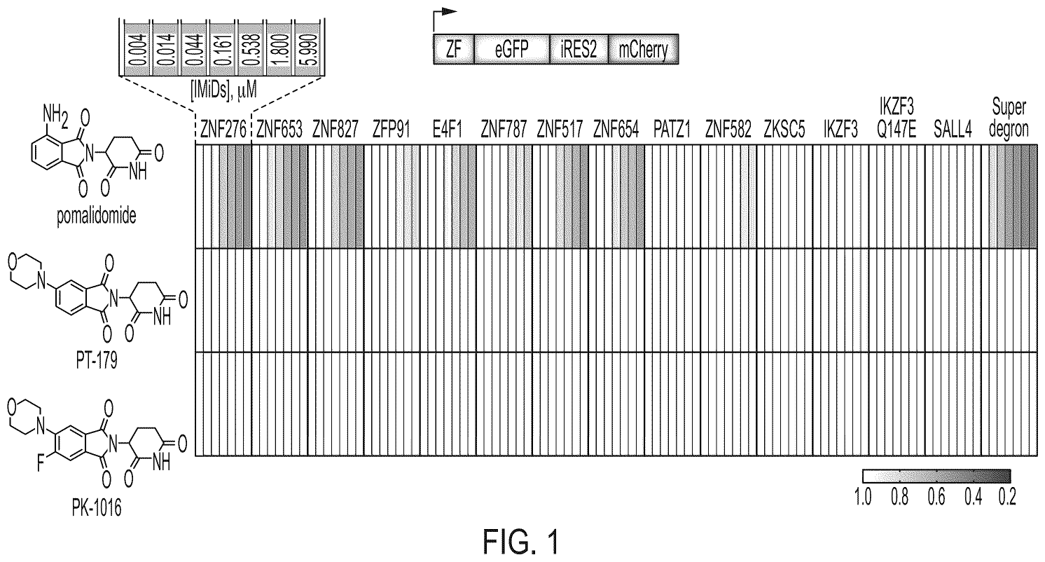

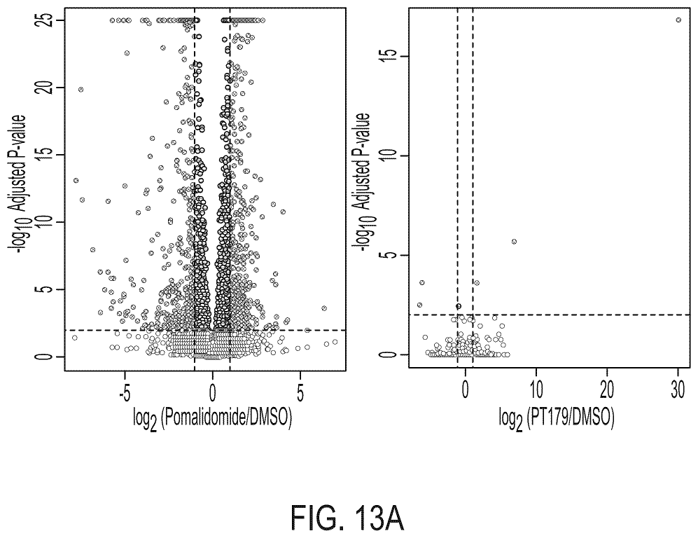

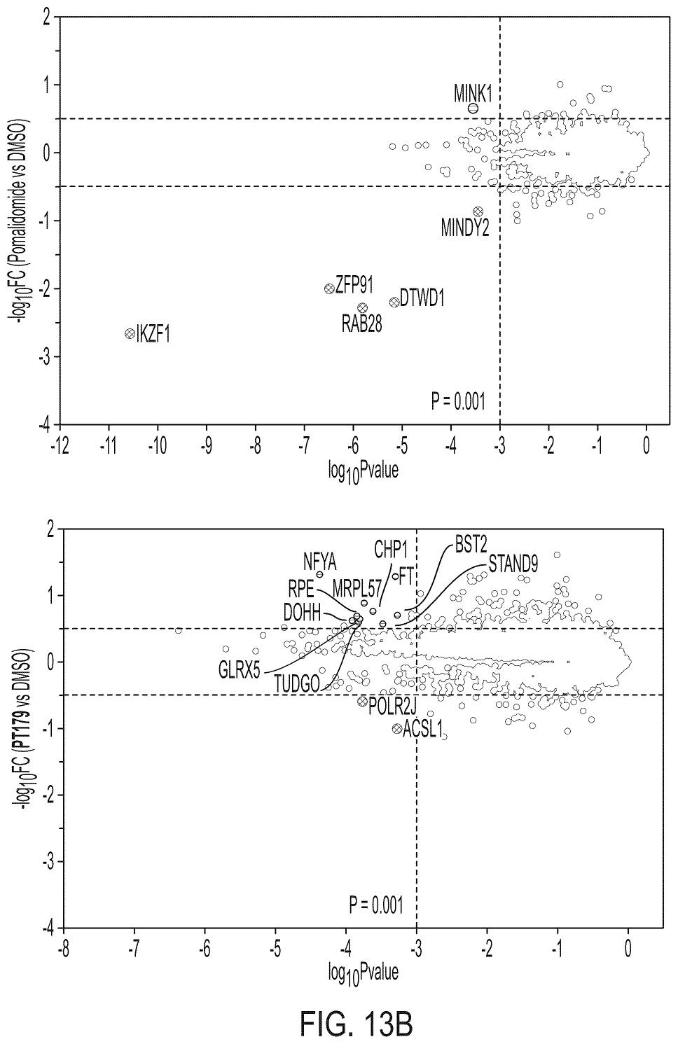

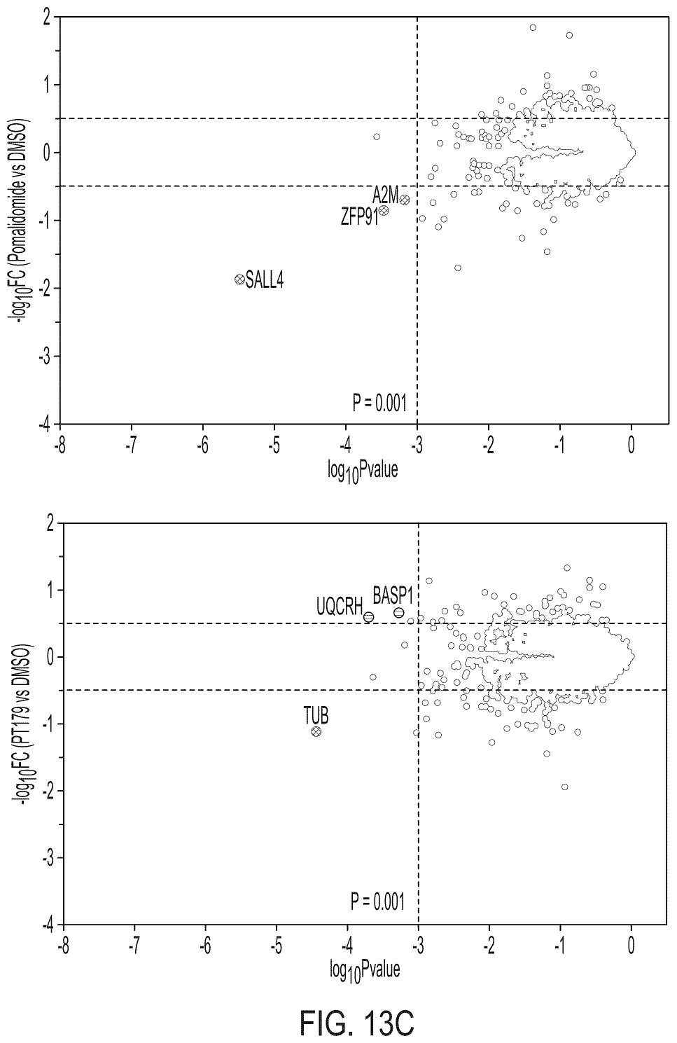

Pomalidomide is a small molecule known to mediate a ternary complex between cereblon and multiple endogenous proteins, several being transcription factors. Other small molecules, VS-777, PT-179, and PK-1016, contain phthalimide substitutions relative to pomalidomide that disrupt the interaction between the super degron and the cereblon-IMiD complex. These small molecules bind cereblon without recruiting endogenous neosubstrates, reducing off-target degradation. For example, it was demonstrated that off-target neosubstrates of pomalidomide are not degraded by molecules like PT-179 and PK-1016 (see FIG. 1, FIG. 13A), which feature a new morpholine substitution at the 5-position of the phthalimide.

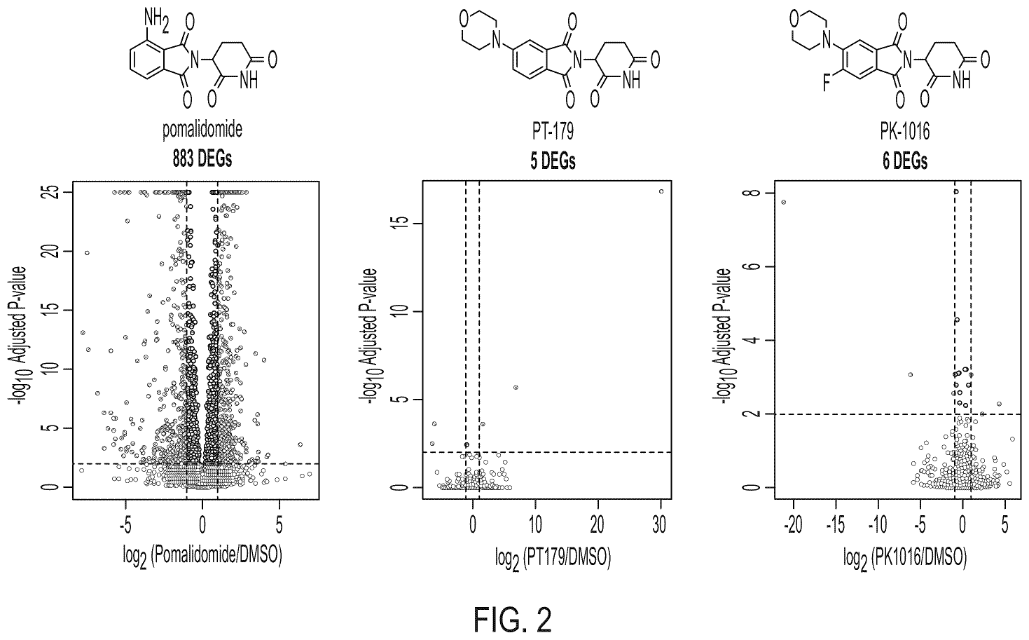

Unbiased off-target profiling of select compounds was utilized to develop the evolved degron tag. An overnight treatment was performed on MM.1S cells with pomalidomide, total RNA was harvested the next day and RNA-seq was run on the samples. Like previously reported findings, hundreds of differentially expressed genes (DEGs) were found relative to a DMSO control (Yamamoto et al., Nature Chemical Biology, 2020) (see FIG. 2). Performing the same experiment and analysis for PT-179 and PK-1016 marked a reduction in the number of DEGs was found (see FIG. 2), which is consistent with the idea that the phthalimide substitutions disrupt the interactions between cereblon and endogenous proteins that are mediated by pomalidomide and IMiDs alike. The RNA-seq data demonstrated that PT-179 and PK-1016 altered expression of very few transcripts in MM.1S cells.

To enable the directed evolution of super degron, SD0, accessory plasmids carrying CRBN fused to a repressor were constructed and assessed for transcriptional activation levels in the presence of the super degron fused to the E. coli RNA polymerase omega (RpoZ). To assess if the transcriptional activation was sufficient for PACE, a selection phage carrying the RpoZ-super degron fusion was constructed and phage enrichment using a strain carrying the cognate CRBN accessory plasmid and in the presence of 10 μM pomalidomide was observed, whereas in a control experiment lacking pomalidomide, the phage was rapidly lost. These results confirm that the super degron-pomalidomide-CRBN interaction is sufficient for phage enrichment and may enable continuous evolution in PACE.

The small molecules, VS-777, PT-179, PK-1016, which contain substitutions that disrupt the interaction between the super degron and the cereblon-IMiD complex, were used with PACE to evolve the super degron into new degron sequences for small-molecule-inducible tagged protein degradation (see FIG. 3). Successive rounds of PACE and/or PANCE selections were performed using the small molecules, to discover mutations in the super degron that rescued the ternary complex formation with high affinity (see FIG. 4).

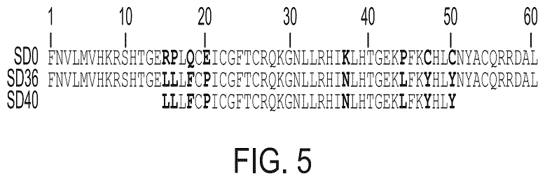

The starting sequence, SD0 (SEQ ID NO: 1) is shown in FIG. 5. Residues that were mutated over the course of successive rounds of PACE are indicated in bold. The final degron variant, SD36 (SEQ ID NO: 37), which came out of the PACE selections, is also shown in FIG. 5 with corresponding mutations bolded. Table 1 shows a summary of amino acid substitutions present in the evolved protein degrons. Truncations of the final degron variant, SD36 were then performed to identify a “minimal” degron sequence and effects of the truncations on SD36 were assessed. The final truncated degron sequence, SD40 (SEQ ID NO: 49), is shown in FIG. 5.

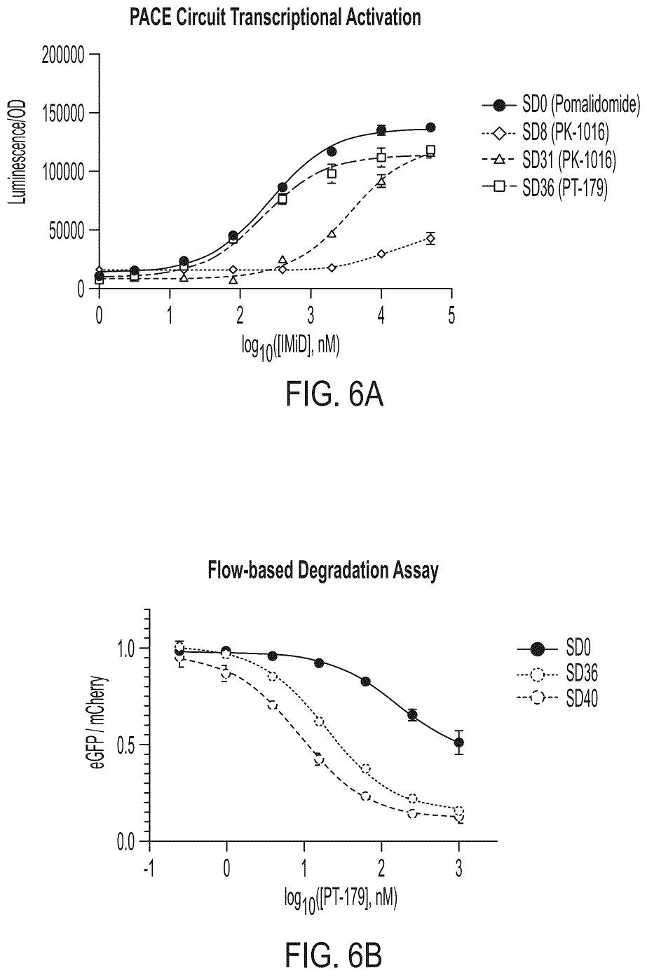

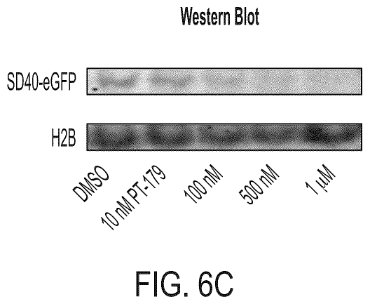

A PACE circuit transcriptional activation assay was performed to assess the activity the evolved protein degrons. Ternary complex formation was measured and the final evolved degron variant, SD36 (SEQ ID NO: 37), showed strong ternary complex formation, as demonstrated in FIG. 6. Increasing SD numbers indicate variants discovered in later stages of selection, with SD0 being the starting degron sequence (SEQ ID NO: 1), from which degron variants were evolved. The final degron variant, SD36 (SEQ ID NO: 37), exhibited a similar dose-response with PT-179 as the starting sequence, SD0 (SEQ ID NO: 1), with pomalidomide. The results show that the evolved degrons respond to a small molecule, PT-179, that has much less biological crosstalk than the canonical small-molecule triggers thalidomide, lenalidomide, or pomalidomide.

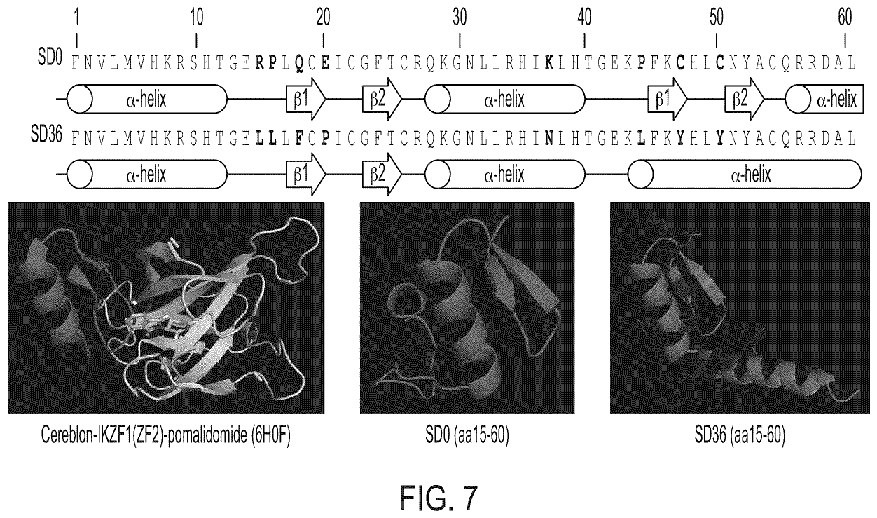

A flow-based degradation assay was then performed using a stably transduced Degron-eGFP-IRES-mCherry construct in HEK 293T cells to determine the ability of the evolved degrons to degrade PT-179 (see FIG. 7). Concentration of PT-179 is shown on the x-axis. Degrons indicated in the key were fused to the N-terminus of eGFP allowing measurement of eGFP:mCherry ratios for assessing degradation. The final degron following truncation, SD40 (SEQ ID NO 49), showed potent degradation in response to overnight PT-179 treatment and marked improvement over the starting sequence, SD0 (SEQ ID NO: 1).

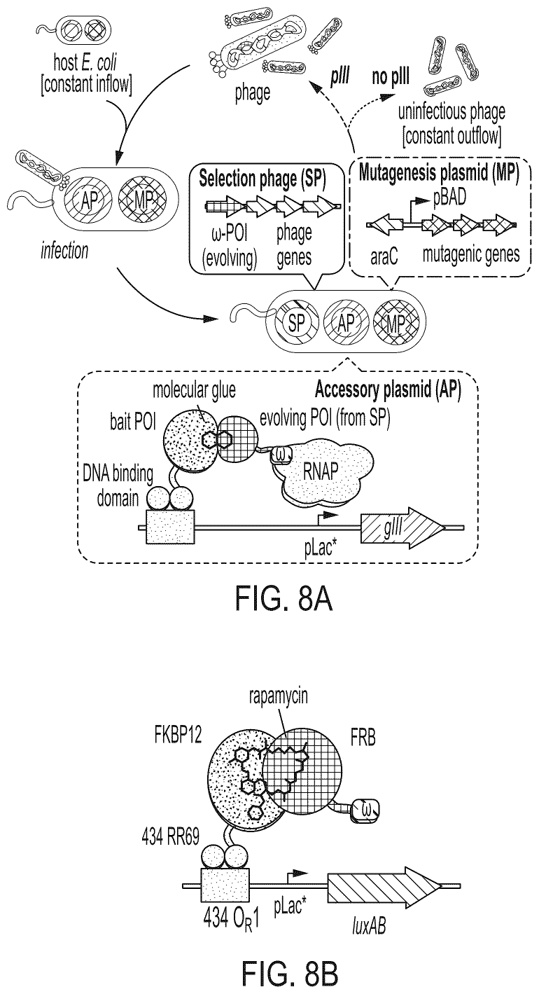

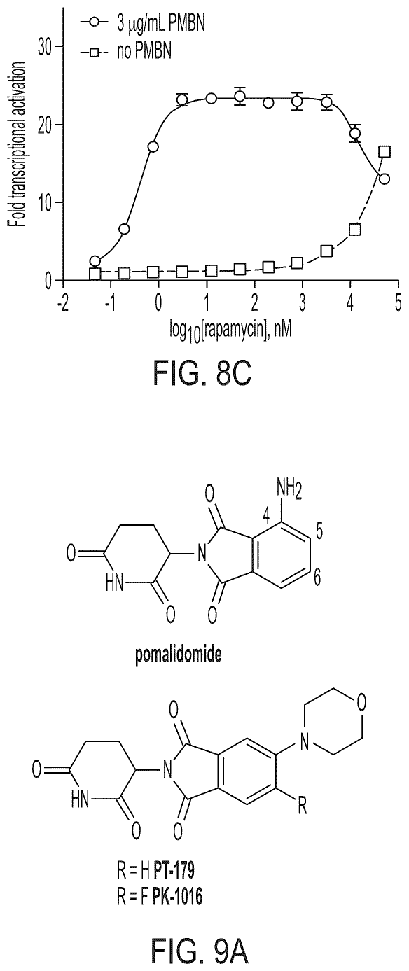

Western blotting was then performed. FIG. 8 shows a western blot visualizing an SD40-eGFP construct (top) and a loading control, H2B (bottom), following overnight treatment with a range of PT-179 concentrations. There is virtually no remaining SD40-eGFP protein when cells are treated with sub-micromolar concentrations of PT-179.

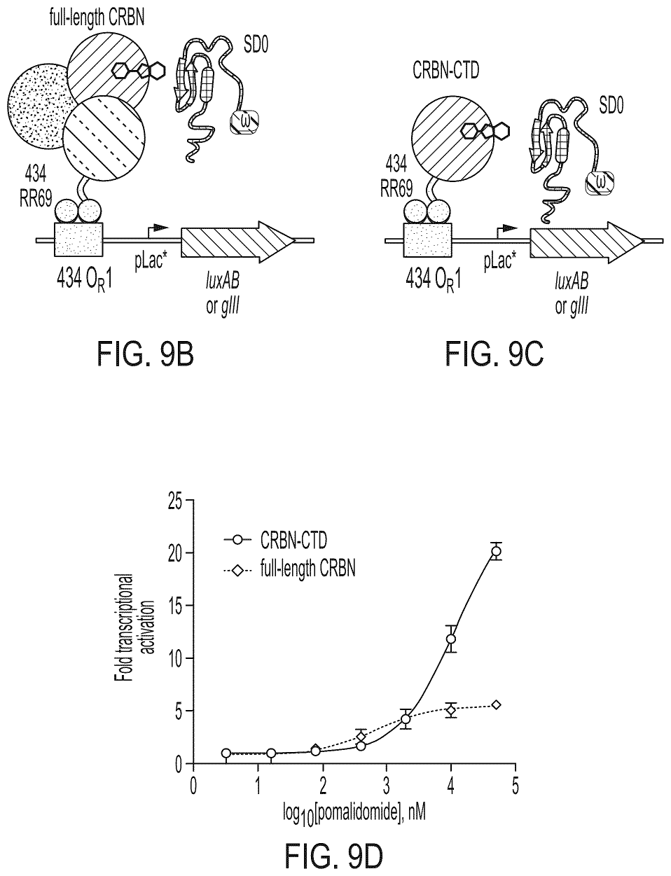

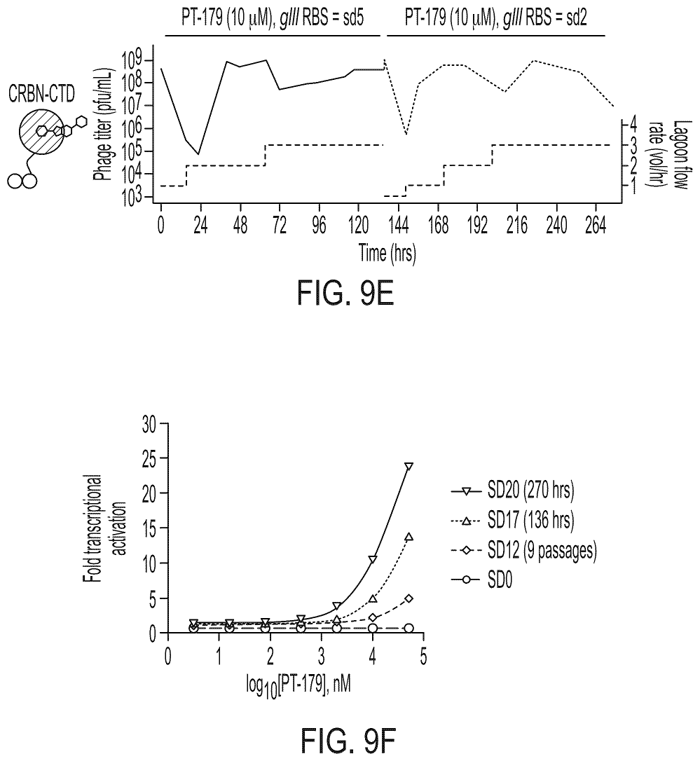

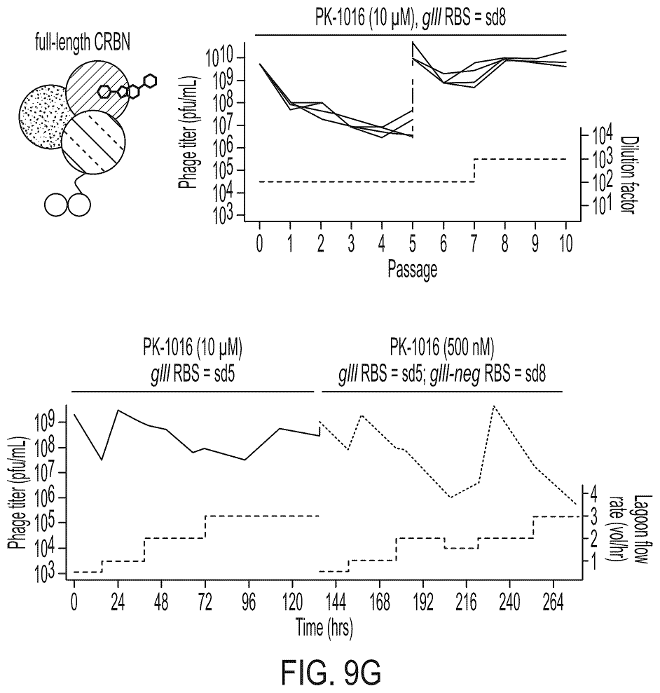

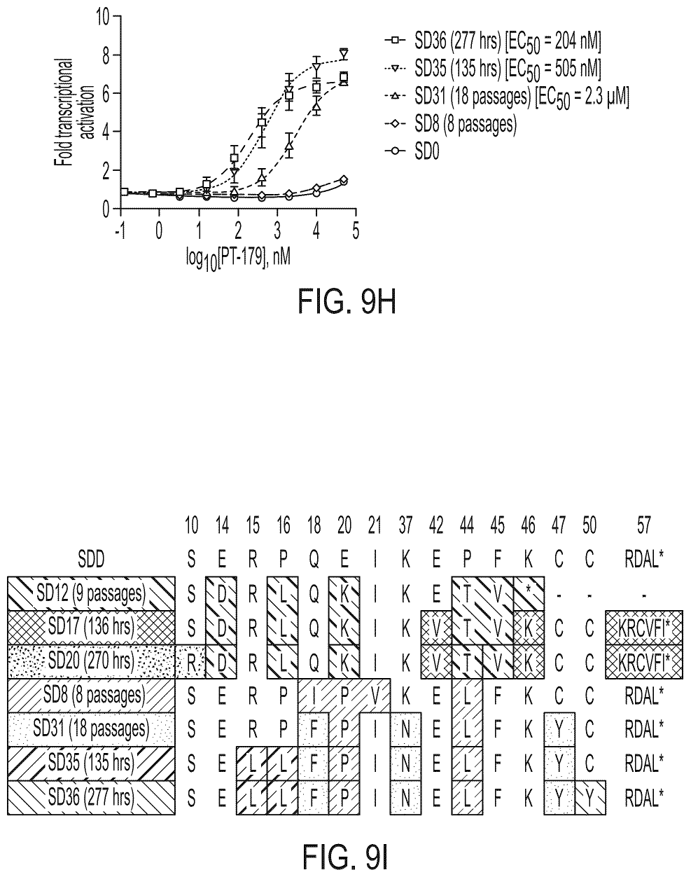

Using the AlphaFold2 platform, the tertiary structure of the evolved degron tag, SD36 was modeled (FIG. 9). There are two clusters of mutations, an N-terminal cluster at residues 15-20 (SD0 numbering) and a C-terminal cluster spanning residues 37-50. The N-terminal cluster is likely accommodating the morpholine bump present on PT-179. The C-terminal cluster has rearranged the whole C-terminal domain of the degron tag to create a new alpha helix. It is expected that this alpha helix is making new contacts with cereblon not present in any native protein degrons, and that these additional contacts are augmenting the overall affinity of the ternary interaction.

TABLE 1
Amino acid substitutions in evolved protein degrons
relative to super degron, SD0 (SEQ ID NO: 1)

| | AA residue | | | | | | | | | | | | | | | | | | |
|---|---|---|---|---|---|---|---|---|---|---|---|---|---|---|---|---|---|---|---|
| | 1 | 3 | 5 | 6 | 7 | 8 | 10 | 12 | 14 | 15 | 16 | 17 | 18 | 20 | 21 | 25 | 28 | 29 | 30 |
| Super | F | V | M | V | H | K | S | T | E | R | P | L | Q | E | I | T | Q | K | G |
| degron | | | | | | | | | | | | | | | | | | | |
| (SD0) | | | | | | | | | | | | | | | | | | | |
| SD1 | | | | | | | | | | | | | | K | | | | | |
| SD2 | | | | | | | | | | | S | | | K | | | | | |
| SD3 | | | | | | | | | | | S | | | K | | | | | |
| SD4 | | | L | | | | | | | | S | | | K | | | | | |
| SD5 | | | | | | | | | | | | F | | K | | | | | |
| SD6 | | | | | | | | | | | | | M | P | V | | | | |
| SD7 | | | | | | | | | | | | | M | P | V | | | | |
| SD8 | | | | | | | | | | | | | I | P | V | | | | |
| SD9 | | | | | | | | | | | | | I | P | V | | | | |
| SD10 | | | | | | | | | | | | | I | P | V | | | | V |
| SD11 | | | | | | | | | | | | | | | | | | | |
| SD12 | | | | | | | | | | | | | | | | | | | |
| SD13 | | | | | | | | | | | L | | | K | | | | | |
| SD14 | | E | | | | | | | | | L | | | K | | | | | |
| SD15 | | | | | | | | | | | S | F | | R | | | | | |
| SD16 | | | | | | | | P | D | | L | | | K | | | | | |
| SD17 | | | | | | | | | D | | L | | | K | | | | | |
| SD18 | | | | G | | E | | | | | S | F | | R | | | | | |
| SD19 | | | | | | E | | | | | S | F | | R | | | | | |
| SD20 | | | | | | | R | | D | | L | | | K | | | E | | |
| SD21 | | | | | | | | | D | | L | | | K | | | E | | |
| SD22 | | | | | | E | | | | | S | F | | R | | | | | |
| SD23 | | | | | | | | | | | S | F | | R | | | | | |
| SD24 | | | | | | | | | | | | F | | R | | | | | |
| SD25 | | | | | | | | | | | L | | | K | | | E | | |
| SD26 | | | | | | | R | | D | | L | | | K | | | E | | |
| SD27 | | | | | | | | | | | L | | | K | | | E | | |
| SD28 | | | | | | | | | | | S | | H | K | | | | | |
| SD29 | | | | | Y | | | | | | S | | H | K | | | | | |
| SD30 | | | | | Y | | | | | | S | | H | K | | M | | | |
| SD31 | | | | | | | | | | | | | F | P | | | | | |
| SD32 | | | | | | | | | | | | | I | P | V | | | | |
| SD33 | | | | | | | | | | | L | | I | P | V | | | | |
| SD34 | | A | | | Y | | | | | | S | | H | K | | M | | | |
| Super | F | V | M | V | H | K | S | T | E | R | P | L | Q | E | I | T | Q | K | G |
| degron | | | | | | | | | | | | | | | | | | | |
| (SD0) | | | | | | | | | | | | | | | | | | | |
| SD35 | | | | | | | | | | L | L | | F | P | | | | | |
| SD36 | | | | | | | | | | L | L | | F | P | | | | | |
| SD36.2 | | | | | | | | | | | L | | F | P | | | | | |
| SD36.3 | | | | | | | | | | L | | | F | P | | | | | |
| SD36.4 | | | | | | | | | | L | L | | | P | | | | | |
| SD36.5 | | | | | | | | | | L | L | | F | | | | | | |
| SD36.6 | | | | | | | | | | L | L | | F | P | | | | | |
| SD36.7 | | | | | | | | | | L | L | | F | P | | | | | |
| SD36.8 | | | | | | | | | | L | L | | F | P | | | | | |
| SD36.9 | | | | | | | | | | L | L | | F | P | | | | | |
| SD37 | | — | — | — | — | — | — | — | — | L | L | | F | P | | | | | |
| SD38 | | | | | | | | | | L | L | | F | P | | | | | |
| SD39 | | — | — | — | — | — | — | — | — | L | L | | F | P | | | | | |
| SD40 | | — | — | — | — | — | — | — | — | L | L | | F | P | | | | | |
| SD41 | | — | — | — | — | — | — | — | — | L | L | | F | P | | | | | |
| SD42 | | — | — | — | — | — | — | — | — | L | L | | F | P | | | | | |
| SD43 | | — | — | — | — | — | — | — | — | L | L | | F | P | | | | | |
| SD44 | | — | — | — | — | — | — | — | — | — | L | | F | P | | | | | |
| SD45 | | | | | | | | | | L | L | | F | P | | | | | |
| SD46 | | | | | | | | | | L | L | | F | P | | | K | | |
| SD47 | | | | | | | | | | L | L | | F | P | | | | | |
| SD48 | | | | G | | | | | D | L | L | | F | P | | | | E | |
| SD49 | L | | | | | R | | | | L | L | | F | P | | | | | |

| | AA residue | | | | | | | | | | | | | | | | | |
|---|---|---|---|---|---|---|---|---|---|---|---|---|---|---|---|---|---|---|
| | 31 | 37 | 40 | 41 | 42 | 44 | 45 | 46 | 47 | 50 | 51 | 53 | 54 | 57 | 58 | 59 | 60 | 61 |
| Super | N | K | T | G | E | P | F | K | C | C | N | A | C | R | D | A | L | — |
| degron | | | | | | | | | | | | | | | | | | |
| (SD0) | | | | | | | | | | | | | | | | | | |
| SD1 | | | | | | L | | | | | | | | | | | | |
| SD2 | | | | | | L | | | | | | | | | | | | |
| SD3 | | | | | V | L | | | | | | | | | | | | |
| SD4 | | | | | V | L | | | | | | | | | | | | |
| SD5 | K | | | | | L | | | | | | | | | | | | |
| SD6 | | | M | | | L | | | | | | | | | | | | |
| SD7 | K | | M | | | L | | | | | | | | | | | | |
| SD8 | | | | | | L | | | | | | | | | | | | |
| SD9 | | | | | | L | | | | | K | | | | | | | |
| SD10 | | | | | | L | | | | | | | | | | | | |
| SD11 | | | | | | T | V | stop | — | — | — | — | — | — | — | — | — | — |
| SD12 | | | | | | | | stop | — | — | — | — | — | — | — | — | — | — |
| SD13 | | | | | | | | | | | | | | | | | | |
| SD14 | | | | | | | | | | | | | | | | | | |
| SD15 | | | | | V | L | | | | | | | | | | | | |
| SD16 | D | | | | V | T | V | | | | | | | K | R | C | F | VI |
| SD17 | | | | | V | T | V | | | | | | | K | R | C | F | VI |
| SD18 | | | | D | V | L | L | | | | | D | | | | | | |
| SD19 | | | | D | V | L | | | | | | | | | | | | |
| SD20 | | | | | V | T | V | | | | | | | K | R | C | F | VI |
| SD21 | | | | | V | T | V | | | | | | | K | R | C | F | VI |
| SD22 | | | | D | V | L | | | | | | D | | | | | | |
| SD23 | | | | D | V | L | | | | | | | | | | | | |
| SD24 | | | | D | V | L | | | | | | | | | | | | |
| SD25 | | | | | V | T | V | | | | | | | K | R | C | F | VI |
| SD26 | | | | | V | T | V | | | | | | | | | | | |
| SD27 | | | | | V | T | V | | | | | | | | | | | |
| SD28 | | | | | V | L | | | | | | | | | | | | |
| SD29 | | | | | V | L | | | | | | | | | | | | |
| SD30 | | | | | V | L | | | | | | | | | | | | |
| SD31 | | N | | | | L | | | Y | | | | | | | | | |
| SD32 | | N | P | | | L | | | Y | | | | | | | | | |
| SD33 | | N | P | | | L | | | Y | | | | | | | | | |
| SD34 | | | | | V | L | | | | | | | | | N | | | |
| Super | N | K | T | G | E | P | F | K | C | C | N | A | C | R | D | A | L | — |
| degron | | | | | | | | | | | | | | | | | | |
| (SD0) | | | | | | | | | | | | | | | | | | |
| SD35 | | N | | | | L | | | Y | | | | | | | | | |
| SD36 | | N | | | | L | | | Y | Y | | | | | | | | |
| SD36.2 | | N | | | | L | | | Y | Y | | | | | | | | |
| SD36.3 | | N | | | | L | | | Y | Y | | | | | | | | |
| SD36.4 | | N | | | | L | | | Y | Y | | | | | | | | |
| SD36.5 | | N | | | | L | | | Y | Y | | | | | | | | |
| SD36.6 | | | | | | L | | | Y | Y | | | | | | | | |
| SD36.7 | | N | | | | | | | Y | Y | | | | | | | | |
| SD36.8 | | N | | | | L | | | | Y | | | | | | | | |
| SD36.9 | | N | | | | L | | | | | | | | | | | | |
| SD37 | | N | | | | L | | | Y | Y | | | | | | | | |
| SD38 | | N | | | | L | | | Y | Y | — | — | — | — | — | — | — | — |
| SD39 | | N | | | | L | | | Y | Y | | — | — | — | — | — | — | — |
| SD40 | | N | | | | L | | | Y | Y | — | — | — | — | — | — | — | — |
| SD41 | | N | | | | L | | | Y | — | — | — | — | — | — | — | — | — |
| SD42 | | N | | | | L | — | — | — | — | — | — | — | — | — | — | — | — |
| SD43 | | N | — | — | — | — | — | — | — | — | — | — | — | — | — | — | — | — |
| SD44 | | N | — | — | — | — | — | — | — | — | — | — | — | — | — | — | — | — |
| SD45 | | N | P | | | L | | | Y | Y | | | | | | | | |
| SD46 | | N | P | | | L | | | Y | Y | | | | | | | | |
| SD47 | | N | P | | | L | | | Y | Y | | | Y | | | | | |
| SD48 | | N | P | | | L | | | Y | R | | | | | | | | |
| SD49 | | N | P | | | L | | | Y | Y | H | | | | | | | |

### Example 2

### A Phage-Assisted Continuous Evolution Circuit for Molecular Glue Complexes

PACE harnesses the short generation time of the M13 E. coli bacteriophage (˜10 minutes) to perform many generations of evolution in a short time period with minimal researcher intervention, speeding laboratory evolution by at least 100-fold (FIG. 8A). Selection phage (SP) harbor all of the genes necessary to produce progeny phage except for gIII, which encodes the phage coat protein pIII, and in its place express the evolving protein of interest (POI). Host cells contain an accessory plasmid (AP) that provides pIII that is conditional on desired activity from the POI. Selection pressure is applied by dilution with fresh host cells either continuously (PACE) or in passages (phage-assisted non-continuous evolution, PANCE) in fixed-volume vessels called lagoons; SP that fail to replicate faster than the rate of dilution wash out of the lagoon. Host cells also express a suite of proteins from a mutagenesis plasmid (MP) that increase the frequency of substitution mutations during SP replication.

A PACE selection was developed that links pIII expression to molecular glue ternary complex formation (MG-PACE), in which a specified protein-protein binding event recruits RNA polymerase to initiate transcription of a reporter gene. A selection system responsive to rapamycin was designed, which induces dimerization of FKBP12 and FRB (FKBP12-rapamycin-binding fragment of mTOR). FKBP12 was fused to the DNA-binding protein RR69, an engineered single-chain variant of the 434 phage repressor (FIG. 8B). The cognate 434 phage operator sequence OR 1 was placed upstream of a pLac-derived promoter that was previously optimized for minimal background transcription in bacterial hybrid circuits. Finally, FRB was fused to the small ω-subunit of the E. coli RNA polymerase. Rapamycin-induced binding of FKBP12 and FRB recruited the full RNA polymerase to pLac, driving expression of gIII or a luciferase reporter luxAB (FIG. 8B).

Luciferase expression was detectable only at high concentrations of rapamycin (FIG. 8C). Coadministration of polymyxin B nonapeptide (PMBN), a polycationic small molecule that permeabilizes the E. coli outer membrane, produced a robust rapamycin dose-response curve. Rapamycin can bind FRB without FKBP12 (K_{D}=26 μM)*, explaining the emergence of a hook effect at high concentrations. Under permeabilizing conditions, the MG-PACE circuit accurately reproduced the entire three-body binding curve predicted for FKBP12·rapamycin·FRB across a 106-fold concentration range (FIG. 8C), suggesting that assembly of the transcription complex was fast enough to reflect equilibrium binding dynamics of the molecular glue ternary complex. These results indicate that the MG-PACE gene circuit accurately links the affinity of a known molecular glue interaction to gene expression, and can serve as the basis of a PACE selection.

### Bumped IMiD Analogs Degrade Fewer Off-Target Neosubstrates

IMiDs bind a hydrophobic pocket in the C-terminal domain of CRBN, placing their glutarimide ring within the pocket and leaving the phthalimide ring partially exposed. CRBN·IMiD neosubstrates engage both the exposed IMiD phthalimide ring and the surrounding CRBN protein surface. A panel of IMiD analogs featuring substituents at the phthalimide 4- and 5-position was synthesized to identify motifs that sterically block native neosubstrate engagement, thereby minimizing the ability of these analogs to induce off-target protein degradation. PT-179 (FIG. 9A) was identified as an IMiD analog that did not induce degradation of known pomalidomide neosubstrates.

Multiple myeloma model MM.1S cells were treated with both compounds (i.e., pomalidomide and PT-179) and performed whole-transcriptome sequencing. Pomalidomide induced differential expression of 883 genes, reflecting degradation of key transcription and epigenetic regulators such as IKZF1/3 and ARID2 (FIG. 13A). Treatment with PT-179 resulted in only five differentially expressed genes, a 177-fold decrease in the frequency of gene expression perturbation. Next, KELLY and MOLT4 cells were treated with both compounds and a global proteomic analysis was performed. In MOLT4 cells pomalidomide induced significant downregulation of several previously identified neosubstrates, such as IKZF1 and ZFP91, while PT-179 did not downregulate a single protein (FIG. 13B). In KELLY cells, pomalidomide induced robust downregulation of the developmental transcription factor SALL4, while PT-179 exhibited no significant SALL4 depletion (FIG. 13C). Taken together, these results demonstrated that PT-179 causes degradation of far fewer off-target neosubstrates than pomalidomide.

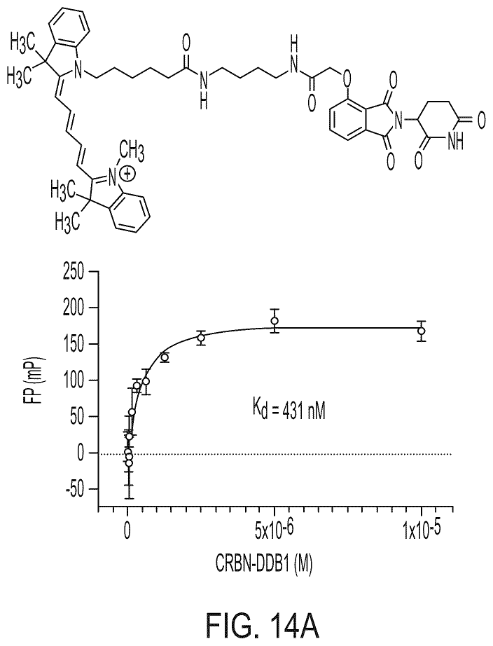

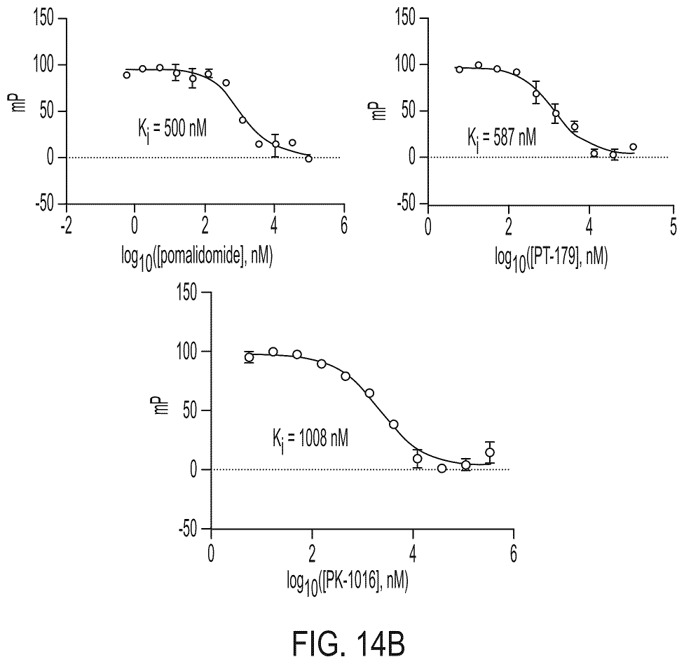

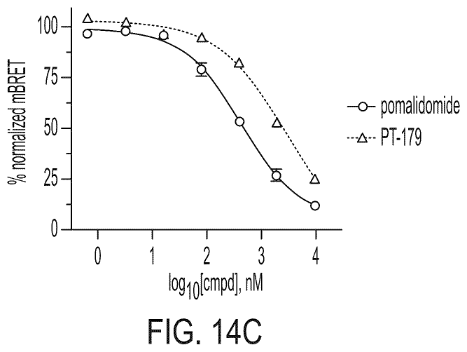

To confirm that PT-179 still binds CRBN, CRBN was purified in complex with its adaptor protein DDB1 and measured its affinity for PT-179 by competitive fluorescence anisotropy. PT-179 binds CRBN with a dissociation constant of 587 nM, similar to the affinity measured for pomalidomide (K_{D}=500 nM, FIGS. 14A-14C). Competitive CRBN engagement assays were performed by bioluminescence resonance energy transfer (BRET) from an ectopically-expressed CRBN-NanoLuc fusion to a fluorescent IMiD conjugate in human cells. These assays revealed that PT-179 engages CRBN in HEK293T and U2OS cells with 7-fold lower potency than pomalidomide (FIG. 14C). Decreased CRBN occupancy could be helpful to reduce any potential interference with CRBN's native function in protein homeostasis, provided it could still evolve potent PT-179-responsive degrons.

Evolution of Zinc Finger Variants that Engage CRBN Bound to Bumped IMiD Analogs

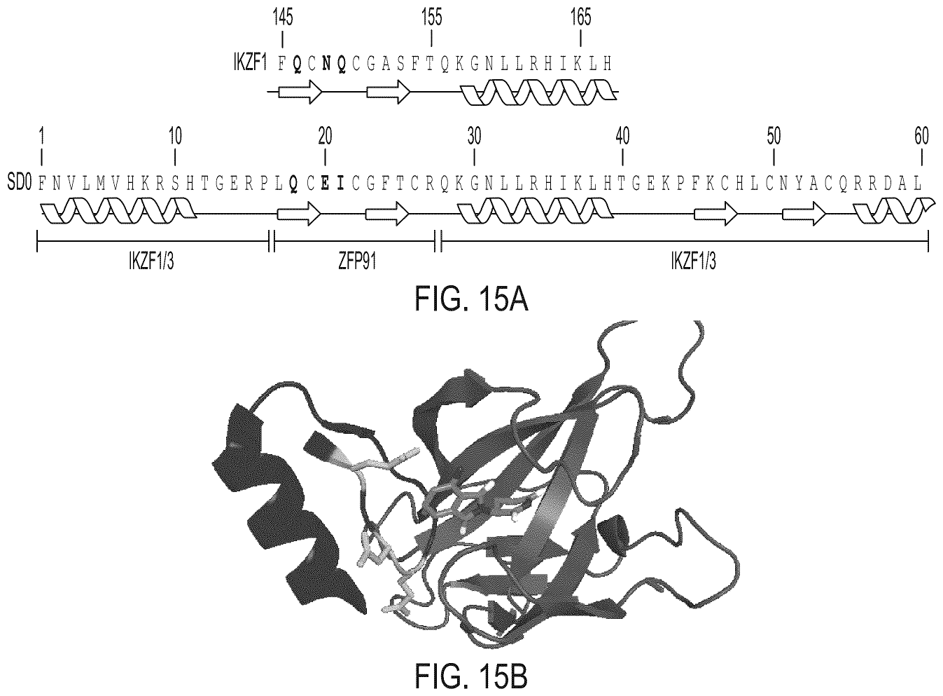

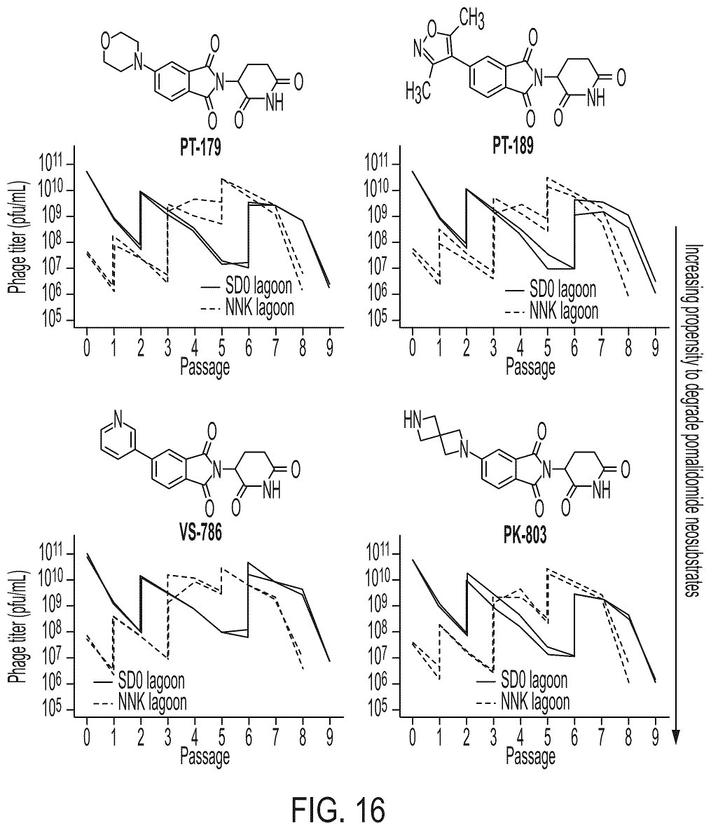

To evolve zinc fingers that engage PT-179-bound CRBN, a MG-PACE circuit was constructed using the CRBN·IMiD·ZF molecular glue complex. As a starting zinc finger, a 60-amino acid chimera comprised of IMiD neosubstrates IKZF1 and ZFP91 was selected that was previously developed as a potent IMID-responsive super degron, hereafter referred to as SD0 (FIG. 15A, Table 1). CRBN was translationally fused to the DNA-binding protein RR69 and fused SD0 to the RNAP ω-subunit, then transcriptional activation was measured by luminescence (FIG. 9B). Pomalidomide elicited a sigmoidal dose-response curve (EC₅₀=686 nM), but with only 6-fold maximal transcriptional activation (FIG. 9D). Anchoring the circuit with the CRBN C-terminal domain (CTD) alone, which contains the IMiD binding pocket, was also explored (FIG. 9C). The CRBN-CTD MG-PACE circuit exhibited 20-fold transcriptional activation at the highest dose, albeit with a rightward shift of the dose-response curve reflecting a decrease in affinity of pomalidomide toward CRBN-CTD, SD0 toward CRBN-CTD·pomalidomide, or both (FIG. 9D). The difference in maximum circuit activation was attributed to poor expression of full-length CRBN in E. coli. The CRBN-CTD MG-PACE circuit was initially used, with the possibility that higher activation would better support weak-binding SD0 variants in the early stages of evolution.

However, attempts to evolve SD0 to bind CRBN-CTD·PT-179 repeatedly failed, suggesting that too many mutations were needed to generate SD0 variants capable of supporting phage propagation. In order to bridge this mutational gap, the CRBN·pomalidomide·IKZF1 co-crystal structure was revisited and three residues were identified as important for accommodating bumped IMiD analogs (FIG. 15B). A library of phage encoding all possible mutations at the three corresponding positions in SD0 (Q18, E20, and I21) was cloned, but variants that conferred PT-179-dependent phage propagation could not be obtained.

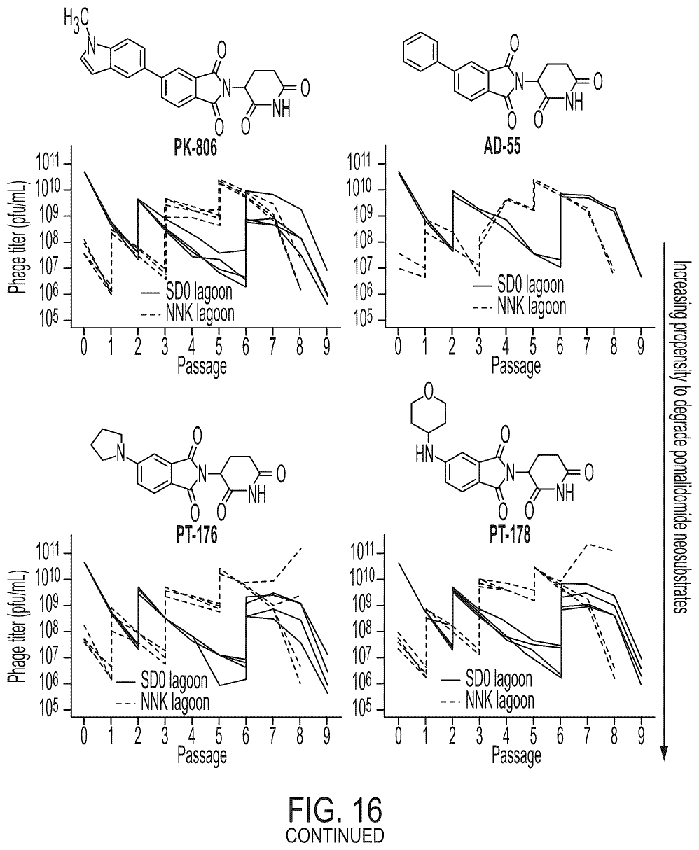

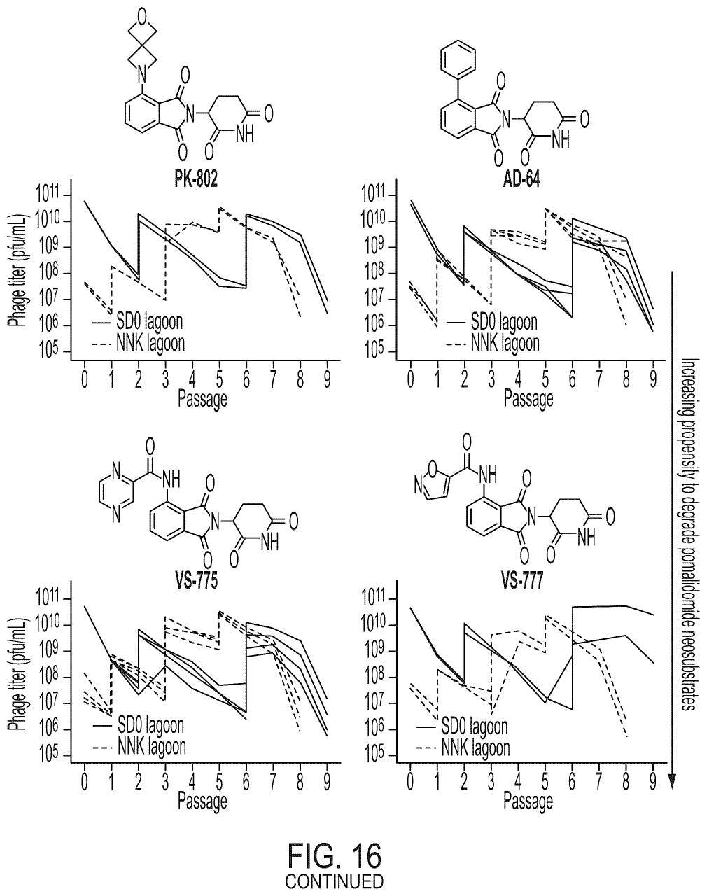

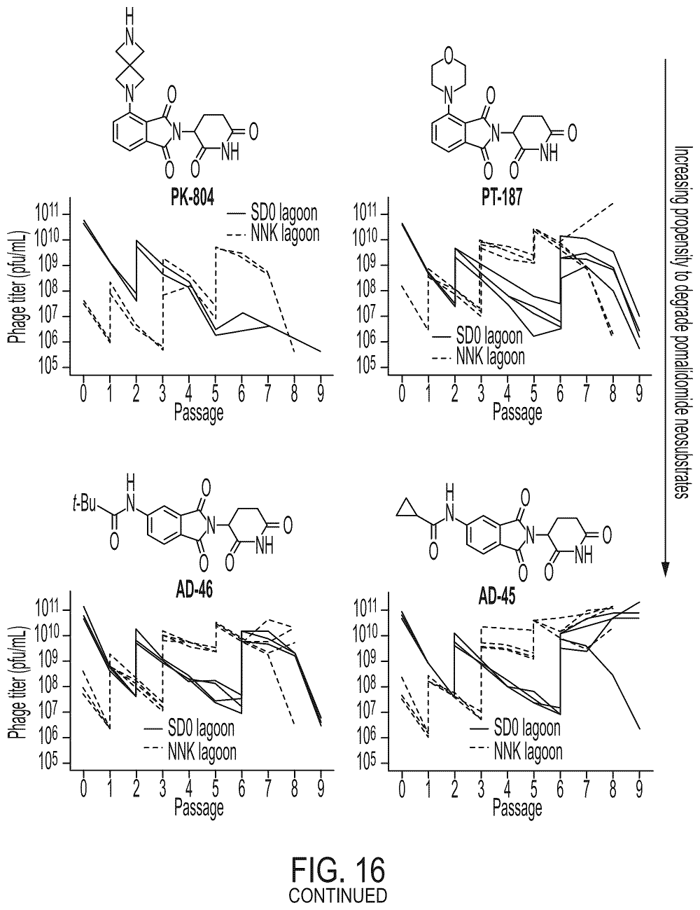

It was possible that IMiD analogs with smaller or less disruptive substituents could serve as evolutionary stepping stones that would challenge SD0 to accommodate IMiD phthalimide-ring substitution but require fewer mutations to reach initial activity. Dozens of substituted IMiD analogs were identified that exhibited only partial disruption of neosubstrate degradation. A panel of 16 IMiD analogs encompassing a range of neosubstrate degradation propensities selected and 96 simultaneous PANCE experiments were conducted (FIG. 16). Though slower than PACE, PANCE is easily parallelized and prevents washout of weakly propagating phage. Each lagoon was seeded with phage encoding either SD0 or the SD0 library and PANCE passages were conducted in media supplemented with a target IMiD analog.

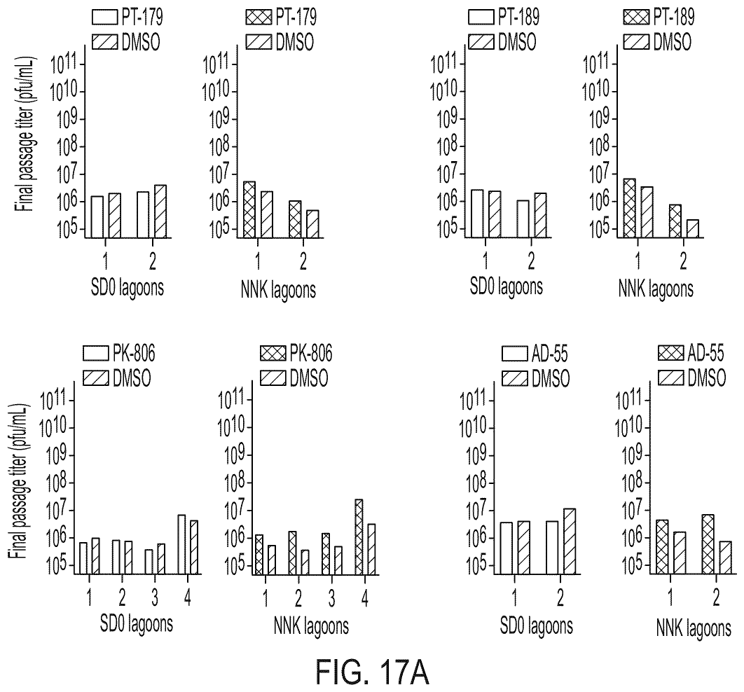

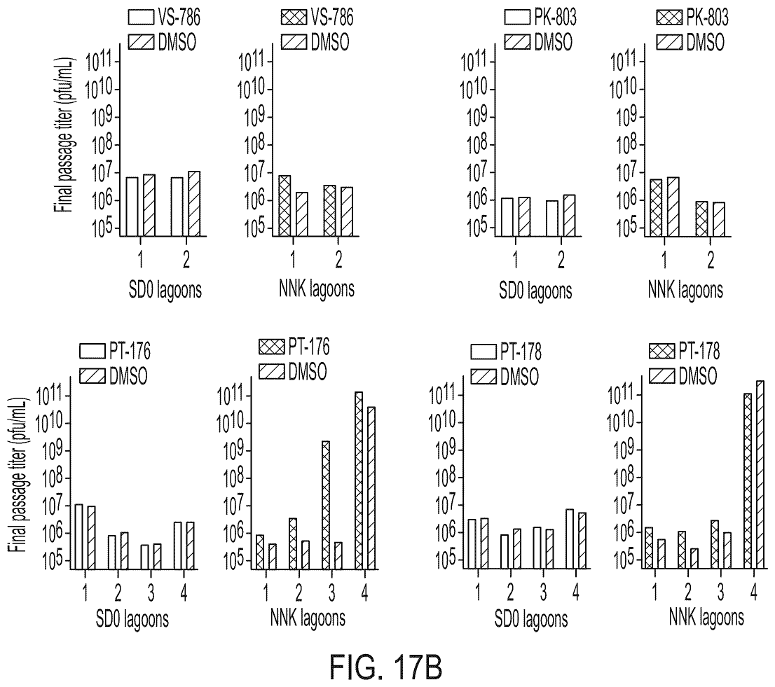

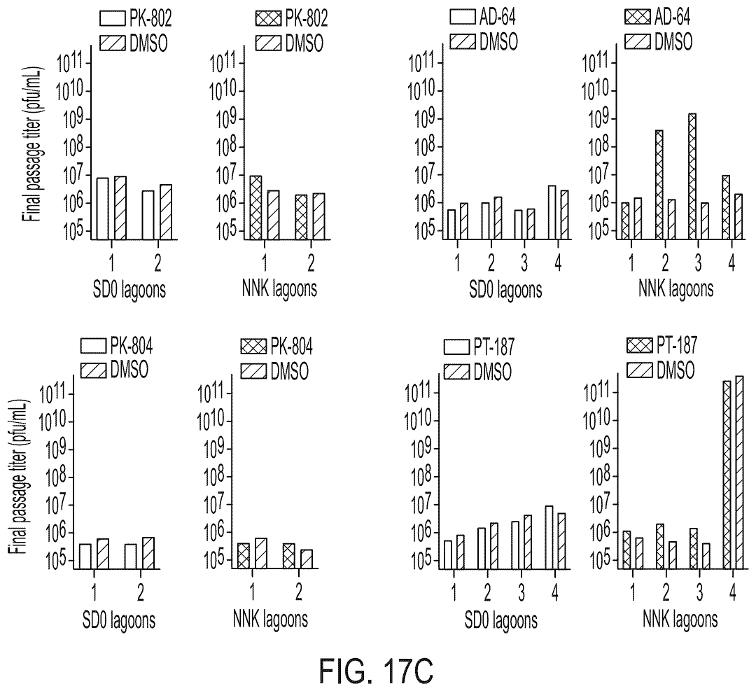

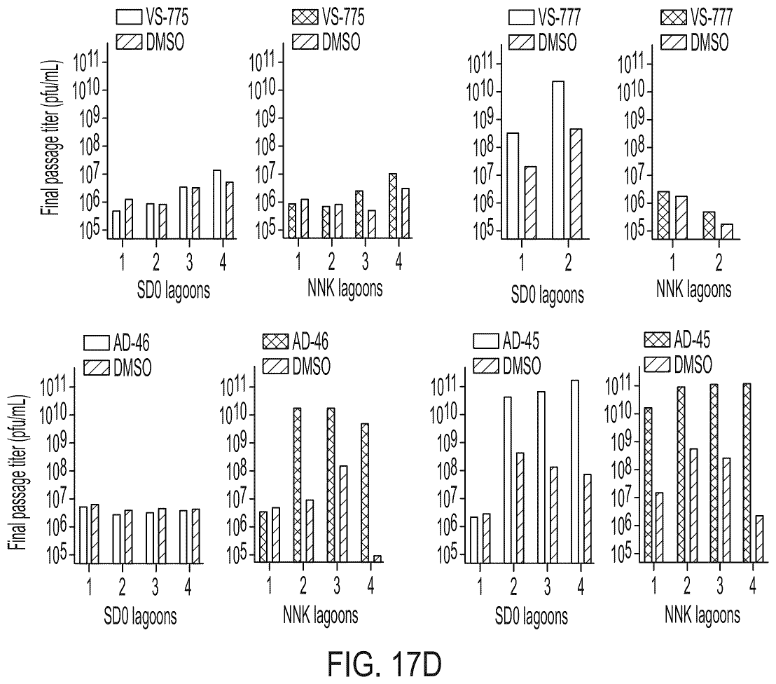

After 8-9 passages at an average overall dilution of 10¹⁵-10¹⁷-fold, 15 pools of phage (10 originating from SD0 library phage, 5 from SD0 phage) that propagated strongly in the presence of their target IMiD analog were obtained (FIGS. 17A-17D). Successful evolutions occurred in lagoons supplemented with IMiD analogs that have the highest propensity to degrade pomalidomide neosubstrates. SD0 variants encoded in surviving phage elicit dose-dependent luciferase expression from the CRBN-CTD MG-PACE circuit in response to their respective stepping stone IMiD analogs and to a lesser extent in response to PT-179 (FIGS. 17A-17D). These results indicate that evolution with IMiD analog stepping stones succeeded in producing SD0 variants that bind PT-179-bound CRBN-CTD.

From these hits, two evolutionary trajectories were explored. First, phage encoding variant SD12 (see Table 1) was subjected to 270 hours of PACE in media supplemented with 10 μM PT-179 (FIG. 9E). Selection stringency was increased by decreasing the strength of the ribosome binding site governing gIll translation, requiring more transcription events from the MG-PACE hybrid promoter to sustain adequate pIII production. Surviving phage converged on variant SD20 which featured five substitutions and three frameshifts resulting in mutations to 12 of the original 60 residues in SD0 and the addition of two C-terminal residues (FIG. 9I, Table 1). SD20 induced robust luciferase expression from the CRBN-CTD MG-PACE circuit in response to PT-179 (24-fold activation at 50 μM PT-179), producing a dose-response curve that overlaps with the SD0/pomalidomide dose-response curve (FIG. 9F). These results suggest that SD20 evolved to bind CRBN-CTD·PT-179 with comparable affinity to CRBN-CTD·pomalidomide·SD0.

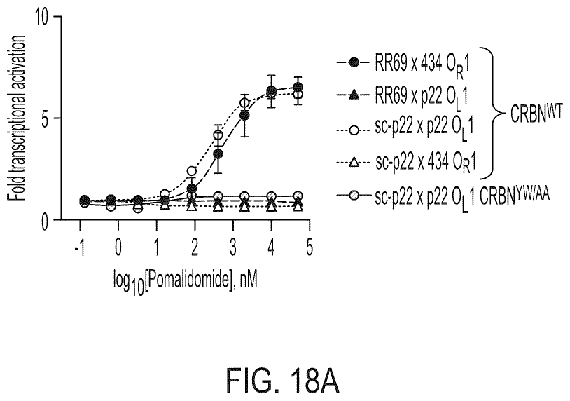

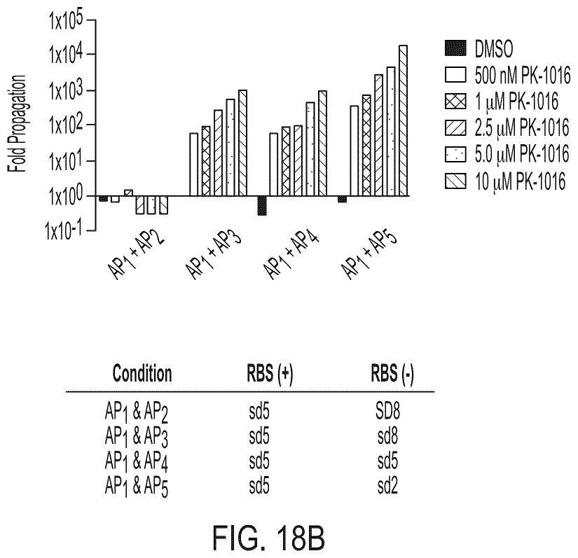

Second, phage encoding variant SD8 (see Table 1) was moved to the full-length CRBN circuit. Ten PANCE passages were conducted (overall 1024-fold dilution) followed by 277 hours of PACE (FIG. 9G). To increase selection stringency, the amount of IMID-analog-bound CRBN was decreased by using PK-1016 (FIG. 9A), a close analog of PT-179 bearing a fluorine at the phthalimide 6-position that binds CRBN 2-fold worse than PT-179 (KD=1008 nM, FIG. 14B), and by decreasing the lagoon concentration of PK-1016 from 10 μM to 500 nM. To discourage evolution of SD8 variants capable of IMID-analog-independent CRBN binding, simultaneous negative selection was performed with a mutant CRBN (Y384A and W386A, CRBN^{YW/AA}) that was deficient in IMiD binding. A new single-chain version of the DNA-binding p22 phage repressor (sc-p22cI), translationally fused sc-p22cI to CRBN^{YW/AA}, was constructed, and placed the p22 OL1 operator upstream of gIII-neg, a truncated form of gIII (FIGS. 18A-18B). SD8 variants that bind CRBN without an IMiD analog would also bind CRBN^{YW/AA}, triggering expression of pIII-neg and production of uninfective progeny phage. The final evolved variant, SD36, has 8 mutated residues (FIG. 9I, Tables 1 and 2) and elicited a potent sigmoidal PT-179 dose-response curve in the full-length CRBN MG-PACE circuit (EC50=204 nM, FIG. 9H).

### Identification and Characterization of a Minimal Degron

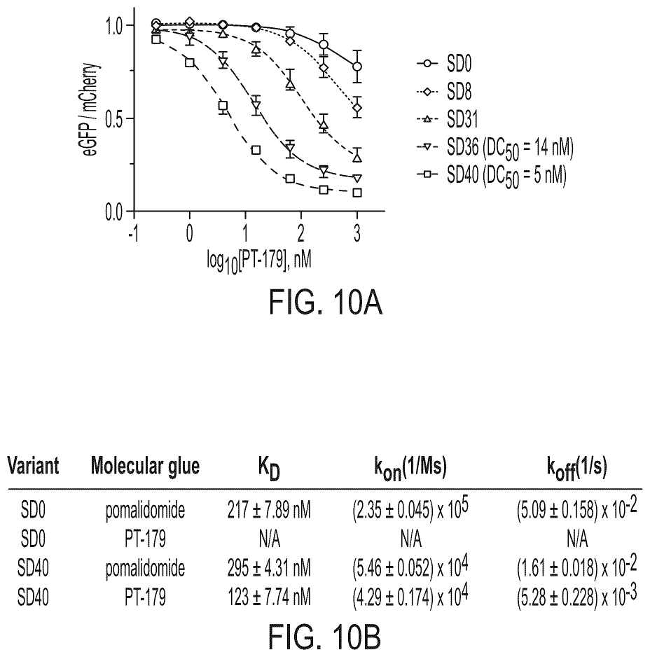

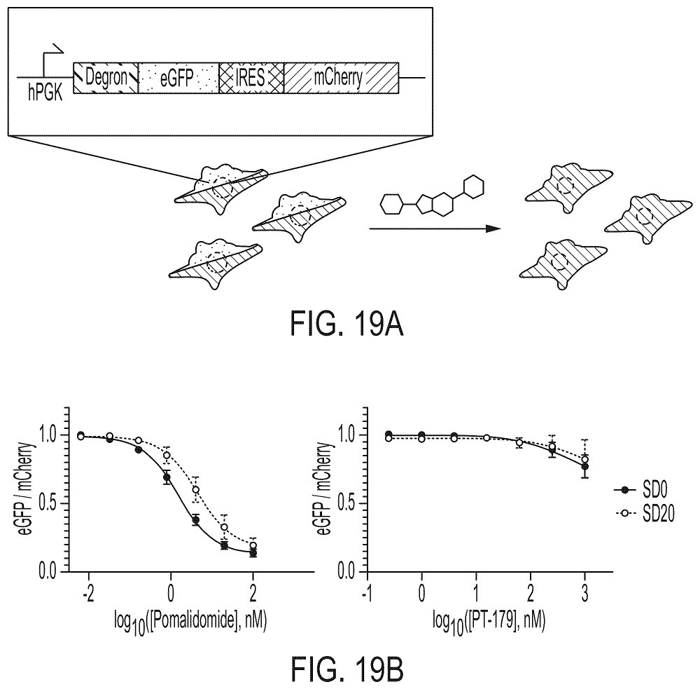

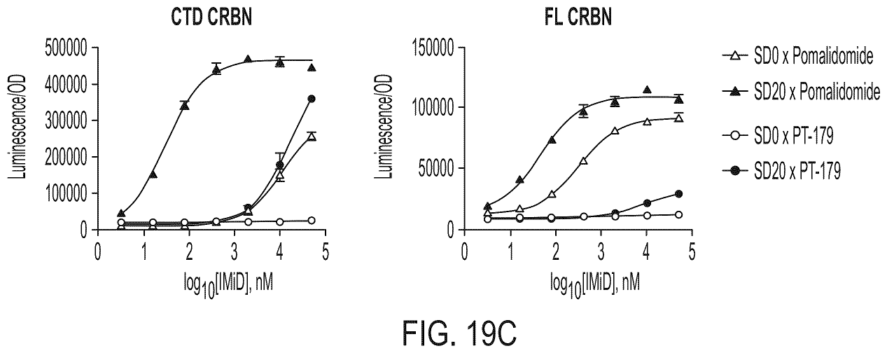

To characterize the evolved degrons, stable HEK293T cell lines expressing degron-tagged eGFP were generated (FIG. 19A). SD20, which evolved on the CRBN-CTD circuit, did not trigger GFP degradation in response to PT-179 (FIG. 19B). In contrast, variants evolved in the full-length CRBN circuit induced PT-179-dependent GFP degradation, with more evolved variants exhibiting greater potency (FIG. 10A). The final variant, SD36, potently degraded GFP in response to PT-179 (DC₅₀=14 nM). These results demonstrate that the full-length CRBN MG-PACE circuit successfully evolved zinc finger degrons that degrade tagged proteins in human cells in response to bumped IMiD analog PT-179.

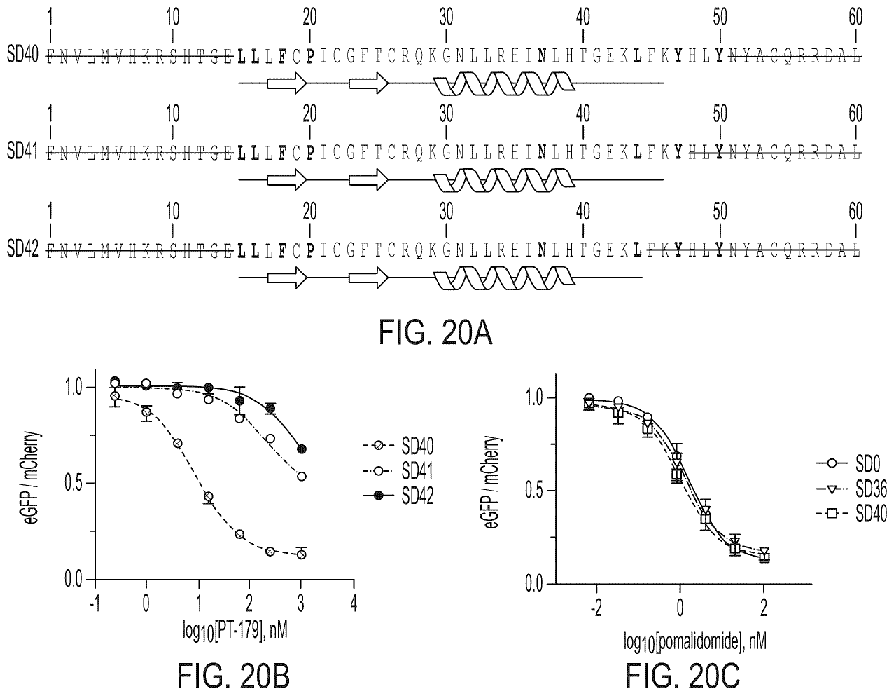

The 8 mutations in SD36 are located in the center of the tag (between residues 15 and 50). Possibly, portions of the N- and C-termini of the degron that went unmutated might be dispensable for its engagement with CRBN·PT-179. The N- and C-terminal ends of SD36 were removed and a 36 amino-acid ‘minimal’ degron, SD40 was identified, that exhibited 2.8-fold enhanced potency for PT-179-induced GFP degradation (DC50=5 nM, FIG. 10A, Table 1). Trimming the degron beyond this point led to a marked reduction in degradation potency (FIGS. 20A-20C).

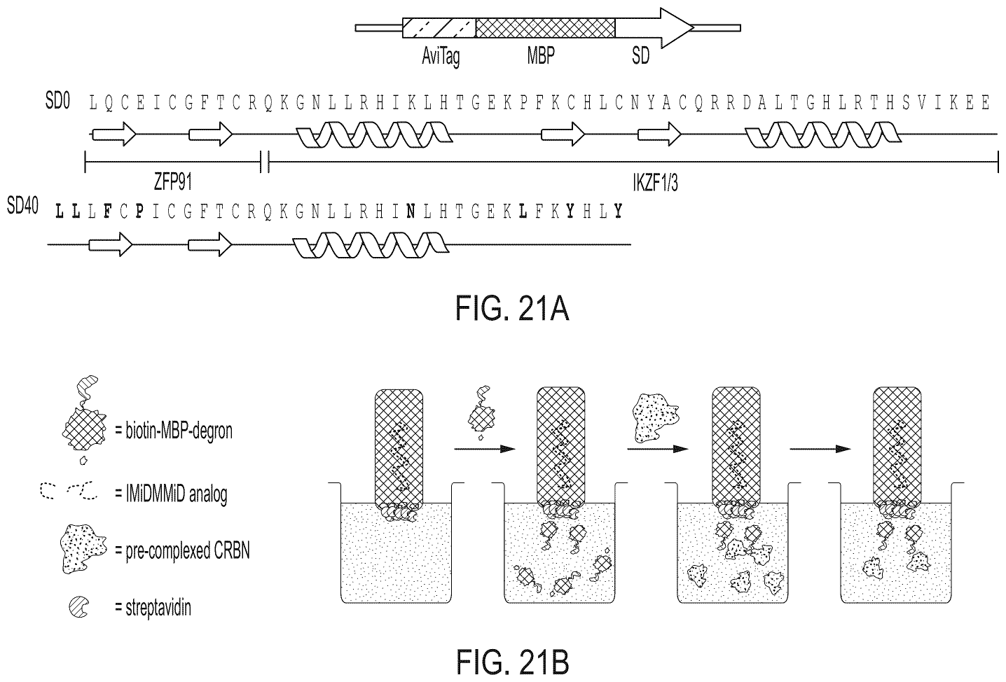

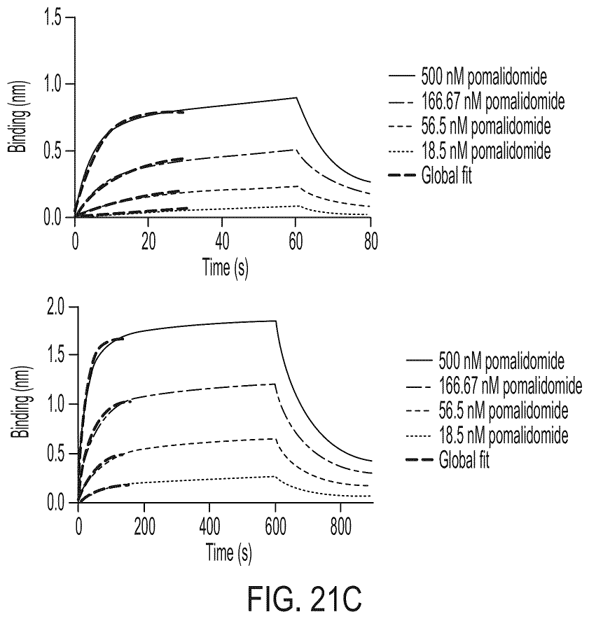

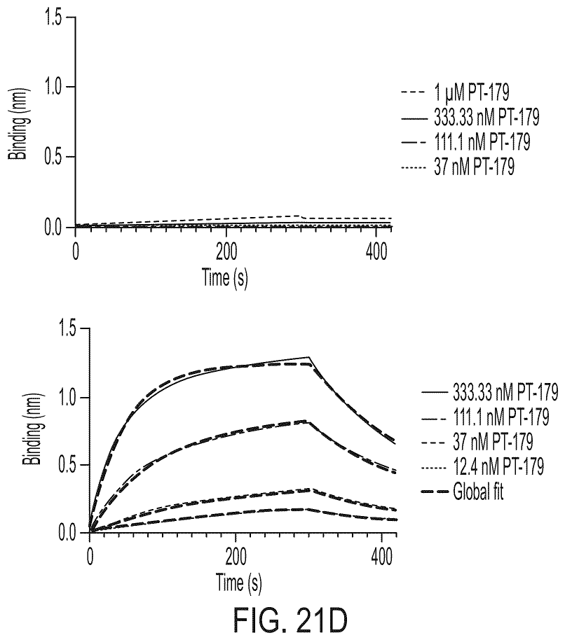

To compare the affinity of the evolved ternary complex CRBN·PT-179·SD40 to that of the original complex CRBN·pomalidomide·SD0, SD40 and SD0 were expressed and purified from E. coli as fusions to maltose binding protein (MBP). A bio-layer interferometry (BLI) with immobilized MBP-degron was conducted to measure association and dissociation rates of DDB1·CRBN precomplexed with either PT-179 or pomalidomide (FIGS. 21A-21D). SD0 bound CRBN·pomalidomide (K_{D}=217 nM) but not CRBN·PT-179 (FIG. 10B). SD40 bound CRBN precomplexed with both molecules with a 2.4-fold preference for PT-179 (CRBN·pomalidomide·SD40 K_{D}=295 nM, CRBN·PT-179·SD40 K_{D}=123 nM). SD40 exhibited slower on- and off-rates relative to SD0 for binding pre-complexed CRBN. Overall, the characterization indicates that MG-PACE successfully evolved a high affinity molecular glue interaction with PT-179-bound CRBN.

### SD40 Induces PT-179-Dependent Degradation of Tagged Proteins in Tissue Culture

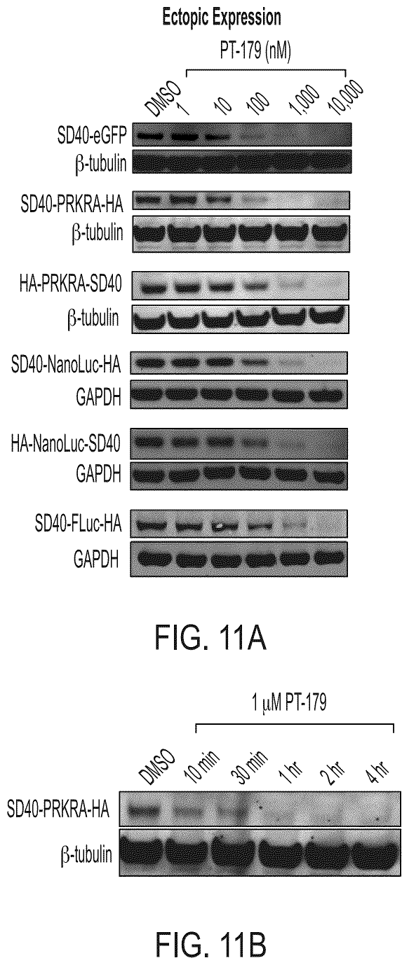

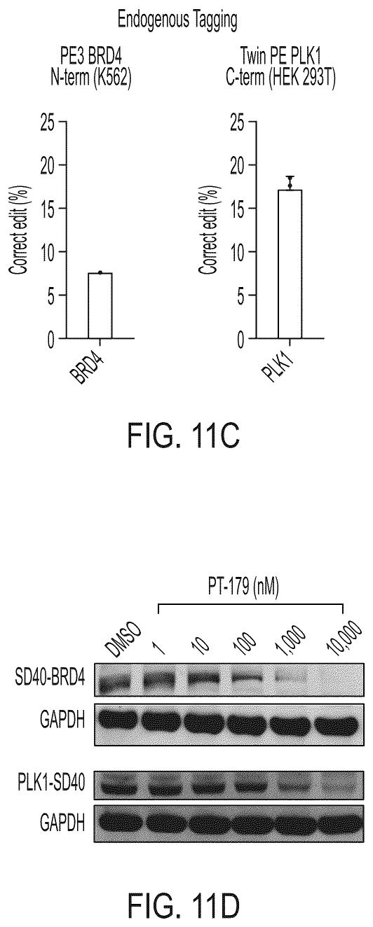

Satisfied with the affinity of the evolved ternary complex and the efficient degradation observed with SD40-eGFP in response to PT-179, the versatility of the evolved degron SD40 was assessed. Five additional stable HEK293T cell lines that ectopically express N- or C-terminal SD40 fusions to Nluc, Fluc, and the kinase activator PRKRA were generated. Robust degradation of each fusion construct in response to PT-179 was observed (FIG. 11A). In a time course experiment, PRKRA degradation onset was observed within 10 minutes of PT-179 administration and near complete degradation within one hour (FIG. 11B). The results demonstrate that SD40 mediates rapid PT-179-triggered degradation of tagged proteins as N- or C-terminal fusions.

### SD40 is Small Enough to be Installed in Target Genes Via Prime Editing

Next, SD40 was installed in target genes in mammalian cells using genome editing methods including nuclease-mediated HDR and prime editing.* Given the small size of SD40, a strategy utilizing SpCas9 RNP with a 200-bp ssODN was tested. Three distinct sites in HEK293T cells were assessed, identifying some sites with up to ˜20% knock-in efficiency. It was assessed whether the degron could be knocked in using prime editing, obviating the need for double-stranded breaks (DSBs) and establishing a less toxic, cleaner, and more broadly applicable method of tagging genes. An 8% knock-in of SD40 at the N-terminus of BRD4 in K562 cells was observed (FIG. 11C). From the pool of edited cells a homozygous line expressing SD40-BRD4 was isolated and it was observed that PT-179 effected robust degradation (FIG. 11D). A twinPE strategy for SD40 knock-in was assessed due to its improved efficiency in performing large insertions. The C-terminus of PLK1 was tagged (17% editing efficiency, FIG. 11C) and a PLK1-SD40 homozygous line was isolated. PLK1 is an essential cell-state regulator, and complete knockdown for 24 hours is cytotoxic. After 2.5 hours of treatment with PT-179, robust PLK1 knockdown and no loss of cell viability was observed (FIG. 11D). Overall, the findings indicate that SD40 is amenable to installation into target genes in human cells by prime editing, enabling facile evaluation of conditional knockdown in an endogenous context.

Evolution of a mCRBN-Compatible Degron Tag

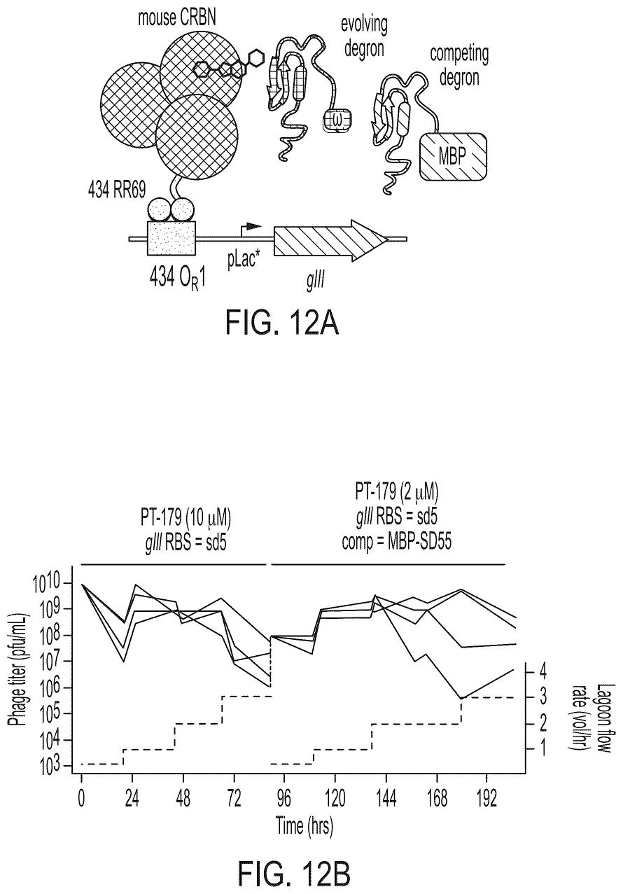

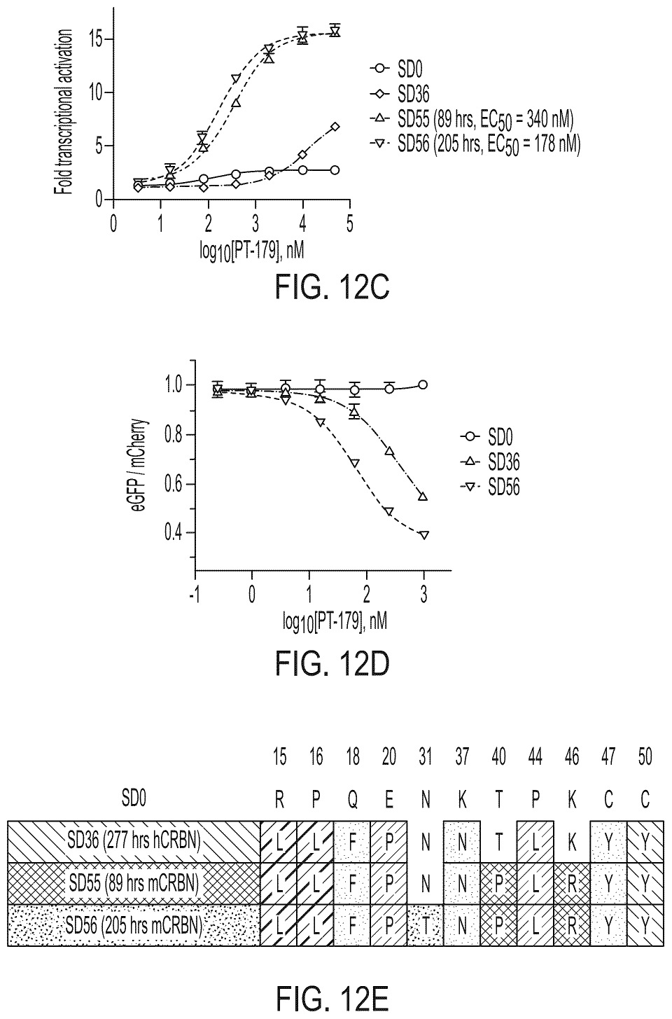

A degron was evolved that is compatible with mouse CRBN (mCRBN) and PT-179. mCRBN contains a V388I mutation at the corresponding interface of human CRBN and its neosubstrates. As a result, mCRBN failed to target these endogenous proteins for effective degradation, potentially explaining the lack of embryopathy in murine models. To determine if MG-PACE could evolve degrons that overcome this bump present on mCRBN and respond to PT-179, a mCRBN MG-PACE circuit was constructed (FIG. 12A) and 205 hours of PACE seeded with SD36-encoding phage was conducted (FIG. 12B). SD0 did not activate the mCRBN circuit, but SD36 showed some affinity for mCRBN·PT-179 (FIG. 12C). It was possible that SD36 engaged mCRBN in a distinct mode that was less susceptible to steric occlusion by the V388I mutation, perhaps via stronger contacts with the N-terminal domain of mCRBN.

After 89 hours of PACE, variant SD55 was identified, with improved activation of the mCRBN MG-PACE circuit (EC₅₀=340 nM, FIG. 12C, Table 2). To increase selection stringency, approaches in antibody engineering were applied and to determine if the expression of a competing degron variant in PACE would demand the evolving degron to develop higher affinity (FIG. 12A). An additional 116 hours in host cells expressing a fusion of MBP to SD55 was conducted and variant SD56 was identified with a 1.9-fold improvement to the EC₅₀ of circuit activation (EC₅₀=178 nM, FIG. 12C, Table 2).

To confirm that MG-PACE produced a PT-179 responsive degron tag that was functional in mouse cells, mouse 3T3 cells were transduced with SD-eGFP-IRES-mCherry constructs for SD0, SD36, and SD56. Following overnight treatment with PT-179, no signs of degradation with SD0-eGFP were observed but an increase in potency from SD36 to SD56 was seen (FIG. 12D). SD56 is a potent PT-179 responsive degron tag in mouse cells (DC₅₀=60 nM).

TABLE 2
Amino acid substitutions in evolved protein degrons
relative to super degron, SD0 (SEQ ID NO: 1)

| | AA residue | | | | | | | | | | |
|---|---|---|---|---|---|---|---|---|---|---|---|
| | 15 | 16 | 18 | 20 | 31 | 37 | 40 | 44 | 46 | 47 | 50 |
| Super | R | P | Q | E | N | K | T | P | K | C | C |
| degron | | | | | | | | | | | |
| (SD0) | | | | | | | | | | | |
| SD36 | L | L | F | P | N | N | T | L | K | Y | Y |
| SD55 | L | L | F | P | N | N | P | L | R | Y | Y |
| SD56 | l | L | F | P | T | N | P | L | R | Y | Y |

### DISCUSSION

Using off-target profiling, IMiD analogs were identified that no longer mediate engagement and degradation of known endogenous neosubstrates of CRBN. A PACE system was developed to enable rapid and continuous evolution of a high affinity molecular glue interaction between CRBN, the aforementioned IMiD analogs, and a ZF-motif. A chemically inducible degron was developed that is 36 amino acids, efficiently inserted at genomic loci, and responsive to a relatively silent small molecule. Moreover, through the mCRBN evolution, it was determined that MG-PACE can overcome unfavorable residue-neosubstrate contacts, allowing for the development of a PT-179 responsive degron tag in mouse cells.

Collectively, these findings indicate that CRBN neosubstrates are highly plastic and can be engineered through PACE to recognize otherwise unfavorable molecular surfaces of CRBN. Given the highly epistatic nature of these ZF-based motifs, individual mutations could contribute little or even be detrimental without additional mutations. Relative to conventional library construction and screening strategies using saturation mutagenesis, PACE offers a high frequency of scattered mutations enabling the identification of beneficial combinations. Multiple mutations spanning the ZF-motif were often identified following PACE/PANCE reflecting these epistatic properties. Additionally, the N-terminal domain of CRBN is critical for evolution of a productive ZF-based degron, most likely due to the role it plays in augmenting the affinity of the interaction.

The MG-PACE platform enabled rapid evolution of molecular glue interactions with IMiD-bound CRBN. The system can be used to assist in engineering of a variety of CRBN-based OFF or ON switches with ligand properties suitable for a given application. Given these developments of counter selection and the added levers of stringency of small molecule concentration and competitive selection, MG-PACE offers the user with various controls on stringency and selectivity profiles. Lastly, the versatility of this system in its ability to recapitulate non-CRBN based molecular glues was displayed, showing that MG-PACE is useful for evolving molecular glue interactions with diverse proteins and ligands.

### Equivalents and Scope

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

### Representative Sequences

(SEQ ID NO: 5)
Amino Acid Sequences:
Super degron (SD0): WT
(SEQ ID NO: 1)
FNVLMVHKRSHTGERPLQCEICGFTCRQKGNLLRHIKLHTGEKPFKCHLCNYACQRRDAL
SD1:
Mutations (relative to SD0): E20K P44L
(SEQ ID NO: 2)
FNVLMVHKRSHTGERPLQCKICGFTCRQKGNLLRHIKLHTGEKLFKCHLCNYACQRRDAL
SD2:
Mutations (relative to SD0): P16S E20K P44L
(SEQ ID NO: 3)
FNVLMVHKRSHTGERSLQCKICGFTCRQKGNLLRHIKLHTGEKLFKCHLCNYACQRRDAL
SD3:
Mutations (relative to SD0): P16S E20K E42V P44L
(SEQ ID NO: 4)
FNVLMVHKRSHTGERSLQCKICGFTCRQKGNLLRHIKLHTGVKLFKCHLCNYACQRRDAL
SD4:
Mutations (relative to SD0): M5L P16S E20K E42V P44L
(SEQ ID NO: 5)
FNVLLVHKRSHTGERSLQCKICGFTCRQKGNLLRHIKLHTGVKLFKCHLCNYACQRRDAL
SD5:
Mutations (relative to SD0): L17F E20K N31K P44L
(SEQ ID NO: 6)
FNVLMVHKRSHTGERPFQCKICGFTCRQKGKLLRHIKLHTGEKLFKCHLCNYACQRRDAL
SD6:
Mutations (relative to SD0): Q18M E20P 121V T40M P44L
(SEQ ID NO: 7)
FNVLMVHKRSHTGERPLMCPVCGFTCRQKGNLLRHIKLHMGEKLFKCHLCNYACQRRDAL
SD7:
Mutations (relative to SD0): Q18M E20P I21V N31K T40M P44L
(SEQ ID NO: 8)
FNVLMVHKRSHTGERPLMCPVCGFTCRQKGKLLRHIKLHMGEKLFKCHLCNYACQRRDAL
SD8:
Mutations (relative to SD0): Q181 E20P I21V P44L
(SEQ ID NO: 9)
FNVLMVHKRSHTGERPLICPVCGFTCRQKGNLLRHIKLHTGEKLFKCHLCNYACQRRDAL
SD9:
Mutations (relative to SD0): Q18I E20P I21V P44L N51K
(SEQ ID NO: 10)
FNVLMVHKRSHTGERPLICPVCGFTCRQKGNLLRHIKLHTGEKLFKCHLCKYACQRRDAL
SD10:
Mutations (relative to SD0): Q181 E20P I21V G30V P44L
(SEQ ID NO: 11)
FNVLMVHKRSHTGERPLICPVCGFTCRQKVNLLRHIKLHTGEKLFKCHLCNYACQRRDAL
SD11:
Mutations (relative to SD0): P44T F45V K46stop
(SEQ ID NO: 12)
FNVLMVHKRSHTGERPLQCEICGFTCRQKGNLLRHIKLHTGEKTV*
SD12:
Mutations (relative to SD0): E14D P16L E20K P44T F45V K46stop
(SEQ ID NO: 13)
FNVLMVHKRSHTGDRLLQCKICGFTCRQKGNLLRHIKLHTGEKTV*
SD13:
Mutations (relative to SD0): P16L E20K
(SEQ ID NO: 14)
FNVLMVHKRSHTGERLLQCKICGFTCRQKGNLLRHIKLHTGEKPFKCHLCNYACQRRDAL
SD14:
Mutations (relative to SD0): V3E P16L E20K
(SEQ ID NO: 15)
FNELMVHKRSHTGERLLQCKICGFTCRQKGNLLRHIKLHTGEKPFKCHLCNYACQRRDAL
SD15:
Mutations (relative to SD0): P16S L17F E20R E42V P44L
(SEQ ID NO: 16)
FNVLMVHKRSHTGERSFQCRICGFTCRQKGNLLRHIKLHTGVKLFKCHLCNYACQRRDAL
SD16:
Mutations (relative to SD0): T12P E14D P16L E20K N31D E42V P44T F45V R57K D58R
A59C L60F *61VI
(SEQ ID NO: 17)
FNVLMVHKRSHPGDRLLQCKICGFTCRQKGDLLRHIKLHTGVKTVKCHLCNYACQRKRCFVI
SD17:
Mutations (relative to SD0): E14D P16L E20K E42V P44T F45V R57K D58R A59C L60F
*61VI
(SEQ ID NO: 18)
FNVLMVHKRSHTGDRLLQCKICGFTCRQKGNLLRHIKLHTGVKTVKCHLCNYACQRKRCFVI
SD18:
Mutations (relative to SD0): V6G K8E P16S L17F E20R G41D E42V P44L F45L A53D
(SEQ ID NO: 19)
FNVLMGHERSHTGERSFQCRICGFTCRQKGNLLRHIKLHTDVKLLKCHLCNYDCQRRDAL
SD19:
Mutations (relative to SD0): K8E P16S L17F E20R G41D E42V P44L
(SEQ ID NO: 20)
FNVLMVHERSHTGERSFQCRICGFTCRQKGNLLRHIKLHTDVKLFKCHLCNYACQRRDAL
SD20:
Mutations (relative to SD0): S10R E14D P16L E20K Q28E E42V P44T F45V R57K D58R
A59C L60F *61VI
(SEQ ID NO: 21)
FNVLMVHKRRHTGDRLLQCKICGFTCREKGNLLRHIKLHTGVKTVKCHLCNYACQRKRCFVI
SD21:
Mutations (relative to SD0): E14D P16L E20K Q28E E42V P44T F45V R57K D58R A59C
L60F *61VI
(SEQ ID NO: 22)
FNVLMVHKRSHTGDRLLQCKICGFTCREKGNLLRHIKLHTGVKTVKCHLCNYACQRKRCFVI
SD22:
Mutations (relative to SD0): K8E P16S L17F E20R G41D E42V P44L A53D
(SEQ ID NO: 23)
FNVLMVHERSHTGERSFQCRICGFTCRQKGNLLRHIKLHTDVKLFKCHLCNYDCQRRDAL
SD23:
Mutations (relative to SD0): P16S L17F E20R G41D E42V P44L
(SEQ ID NO: 24)
FNVLMVHKRSHTGERSFQCRICGFTCRQKGNLLRHIKLHTDVKLFKCHLCNYACQRRDAL
SD24:
Mutations (relative to SD0): L17F E20R G41D E42V P44L
(SEQ ID NO: 25)
FNVLMVHKRSHTGERPFQCRICGFTCRQKGNLLRHIKLHTDVKLFKCHLCNYACQRRDAL
SD25:
Mutations (relative to SD0): P16L E20K Q28E E42V P44T F45V R57K D58R A59C L60F
*61VI
(SEQ ID NO: 26)
FNVLMVHKRSHTGERLLQCKICGFTCREKGNLLRHIKLHTGVKTVKCHLCNYACQRKRCFVI
SD26:
Mutations (relative to SD0): S10R E14D P16L E20K Q28E E42V P44T F45V
(SEQ ID NO: 27)
FNVLMVHKRRHTGDRLLQCKICGFTCREKGNLLRHIKLHTGVKTVKCHLCNYACQRRDAL
SD27:
Mutations (relative to SD0): P16L E20K Q28E E42V P44T F45V
(SEQ ID NO: 28)
FNVLMVHKRSHTGERLLQCKICGFTCREKGNLLRHIKLHTGVKTVKCHLCNYACQRRDAL
SD28:
Mutations (relative to SD0): P16S Q18H E20K E42V P44L
(SEQ ID NO: 29)
FNVLMVHKRSHTGERSLHCKICGFTCRQKGNLLRHIKLHTGVKLFKCHLCNYACQRRDAL
SD29:
Mutations (relative to SD0): H7Y P16S Q18H E20K E42V P44L
(SEQ ID NO: 30)
FNVLMVYKRSHTGERSLHCKICGFTCRQKGNLLRHIKLHTGVKLFKCHLCNYACQRRDAL
SD30:
Mutations (relative to SD0): H7Y P16S Q18H E20K T25M E42V P44L
(SEQ ID NO: 31)
FNVLMVYKRSHTGERSLHCKICGFMCRQKGNLLRHIKLHTGVKLFKCHLCNYACQRRDAL
SD31:
Mutations (relative to SD0): Q18F E20P K37N P44L C47Y
(SEQ ID NO: 32)
FNVLMVHKRSHTGERPLFCPICGFTCRQKGNLLRHINLHTGEKLFKYHLCNYACQRRDAL
SD32:
Mutations (relative to SD0): Q181 E20P I21V K37N T40P P44L C47Y
(SEQ ID NO: 33)
FNVLMVHKRSHTGERPLICPVCGFTCRQKGNLLRHINLHPGEKLFKYHLCNYACQRRDAL
SD33:
Mutations (relative to SD0): P16L Q18I E20P I21V K37N T40P P44L C47Y
(SEQ ID NO: 34)
FNVLMVHKRSHTGERLLICPVCGFTCRQKGNLLRHINLHPGEKLFKYHLCNYACQRRDAL
SD34:
Mutations (relative to SD0): V3A H7Y P16S Q18H E20K T25M E42V P44L D58N
(SEQ ID NO: 35)
FNALMVYKRSHTGERSLHCKICGFMCRQKGNLLRHIKLHTGVKLFKCHLCNYACQRRNAL
SD35:
Mutations (relative to SD0): R15L P16L Q18F E20P K37N P44L C47Y
(SEQ ID NO: 36)
FNVLMVHKRSHTGELLLFCPICGFTCRQKGNLLRHINLHTGEKLFKYHLCNYACQRRDAL
SD36:
Mutations (relative to SD0): R15L P16L Q18F E20P K37N P44L C47Y C50Y
(SEQ ID NO: 37)
FNVLMVHKRSHTGELLLFCPICGFTCRQKGNLLRHINLHTGEKLFKYHLYNYACQRRDAL
SD36.2:
Mutations (relative to SD0): P16L Q18F E20P K37N P44L C47Y C50Y
(SEQ ID NO: 38)
FNVLMVHKRSHTGERLLFCPICGFTCRQKGNLLRHINLHTGEKLFKYHLYNYACQRRDAL
SD36.3:
Mutations (relative to SD0): R15L Q18F E20P K37N P44L C47Y C50Y
(SEQ ID NO: 39)
FNVLMVHKRSHTGELPLFCPICGFTCRQKGNLLRHINLHTGEKLFKYHLYNYACQRRDAL
SD36.4:
Mutations (relative to SD0): R15L P16L E20P K37N P44L C47Y C50Y
(SEQ ID NO: 40)
FNVLMVHKRSHTGELLLQCPICGFTCRQKGNLLRHINLHTGEKLFKYHLYNYACQRRDAL
SD36.5:
Mutations (relative to SD0): R15L P16L Q18F K37N P44L C47Y C50Y
(SEQ ID NO: 41)
FNVLMVHKRSHTGELLLFCEICGFTCRQKGNLLRHINLHTGEKLFKYHLYNYACQRRDAL
SD36.6:
Mutations (relative to SD0): R15L P16L Q18F E20P P44L C47Y C50Y
(SEQ ID NO: 42)
FNVLMVHKRSHTGELLLFCPICGFTCRQKGNLLRHIKLHTGEKLFKYHLYNYACQRRDAL
SD36.7:
Mutations (relative to SD0): R15L P16L Q18F E20P K37N C47Y C50Y
(SEQ ID NO: 43)
FNVLMVHKRSHTGELLLFCPICGFTCRQKGNLLRHINLHTGEKPFKYHLYNYACQRRDAL
SD36.8:
Mutations (relative to SD0): R15L P16L Q18F E20P K37N P44L C50Y
(SEQ ID NO: 44)
FNVLMVHKRSHTGELLLFCPICGFTCRQKGNLLRHINLHTGEKLFKCHLYNYACQRRDAL
SD36.9:
Mutations (relative to SD0): R15L P16L Q18F E20P K37N P44L
(SEQ ID NO: 45)
FNVLMVHKRSHTGELLLFCPICGFTCRQKGNLLRHINLHTGEKLFKYHLCNYACQRRDAL
SD37:
Mutations (relative to SD0): A1-14 R15L P16L Q18F E20P K37N P44L C47Y C50Y
(SEQ ID NO: 46)
LLLFCPICGFTCRQKGNLLRHINLHTGEKLFKYHLYNYACQRRDAL
SD38:
Mutations (relative to SD0): A51-60 R15L P16L Q18F E20P K37N P44L C47Y C50Y
(SEQ ID NO: 47)
FNVLMVHKRSHTGELLLFCPICGFTCRQKGNLLRHINLHTGEKLFKYHLY
SD39:
Mutations (relative to SD0): A1-14, A53-60 R15L P16L Q18F E20P K37N P44L C47Y
C50Y
(SEQ ID NO: 48)
LLLFCPICGFTCRQKGNLLRHINLHTGEKLFKYHLYNY
SD40:
Mutations (relative to SD0): 41-14 A51-60 R15L P16L Q18F E20P K37N P44L C47Y C50Y
(SEQ ID NO: 49)
LLLFCPICGFTCRQKGNLLRHINLHTGEKLFKYHLY
SD41:
Mutations (relative to SD0): A1-14 A48-60 R15L P16L Q18F E20P K37N P44L C47Y
(SEQ ID NO: 50)
LLLFCPICGFTCRQKGNLLRHINLHTGEKLFKY
SD42:
Mutations (relative to SD0): 41-14 A45-60 R15L P16L Q18F E20P K37N P44L
(SEQ ID NO: 51)
LLLFCPICGFTCRQKGNLLRHINLHTGEKL
SD43:
Mutations (relative to SD0): A1-14 A40-60 R15L P16L Q18F E20P K37N
(SEQ ID NO: 52)
LLLFCPICGFTCRQKGNLLRHINLH
SD44:
Mutations (relative to SD0): 41-15 A40-60 P16L Q18F E20P K37N
(SEQ ID NO: 53)
LLFCPICGFTCRQKGNLLRHINLH
SD45:
Mutations (relative to SD0): R15L, P16L, Q18F, E20P, K37N, T40P, P44L, C47Y, C50Y
(SEQ ID NO: 54)
FNVLMVHKRSHTGELLLFCPICGFTCRQKGNLLRHINLHPGEKLFKYHLYNYACQRRDAL
SD46:
Mutations (relative to SD0): R15L, P16L, Q18F, E20P, Q28K, K37N, T40P, P44L, C47Y,
C50Y
(SEQ ID NO: 55)
FNVLMVHKRSHTGELLLFCPICGFTCRKKGNLLRHINLHPGEKLFKYHLYNYACQRRDAL
SD47:
Mutations (relative to SD0): R15L, P16L, Q18F, E20P, K37N, T40P, P44M, C47Y, C50Y,
C54Y
(SEQ ID NO: 56)
FNVLMVHKRSHTGELLLFCPICGFTCRQKGNLLRHINLHPGEKMFKYHLYNYAYQRRDAL
SD48:
Mutations (relative to SD0): V6G, E14D, R15L, P16L, Q18F, E20P, K29E, K37N, T40P,
P44L, C47Y, C50R
(SEQ ID NO: 57)
FNVLMGHKRSHTGDLLLFCPICGFTCRQEGNLLRHINLHPGEKLFKYHLRNYACQRRDAL
SD49:
Mutations (relative to SD0): FIL, K8R, R15L, P16L, Q18F, E20P, K37N, T40P, P44L,
C47Y, C50Y, N51H
(SEQ ID NO: 58)
LNVLMVHRRSHTGELLLFCPICGFTCRQKGNLLRHINLHPGEKLFKYHLYHYACQRRDAL
SD55:
Mutations (relative to SD0): R15L P16L Q18F E20P K37N T40P P44L K46R C47Y C50Y
(SEQ ID NO: 124)
FNVLMVHKRSHTGELLLFCPICGFTCRQKGNLLRHINLHPGEKLFRYHLYNYACQRRDAL
SD56:
Mutations (relative to SD0): R15L P16L Q18F E20P N31T K37N T40P P44L K46R C47Y
C50Y
(SEQ ID NO: 125)
FNVLMVHKRSHTGELLLFCPICGFTCRQKGTLLRHINLHPGEKLFRYHLYNYACQRRDAL
Nucleic Acid Sequences
SD0
Codon-optimized for mammalian expression:
(SEQ ID NO: 96)
TTCAATGTTCTGATGGTTCATAAACGGTCCCACACCGGCGAAAGGCCCTTGCAAT
GTGAGATATGCGGCTTCACTTGTCGCCAAAAAGGTAACCTGCTTAGACACATCAA
ACTTCATACTGGCGAGAAACCTTTCAAATGTCATTTGTGCAATTATGCCTGTCAA
AGACGCGACGCACTG
Codon-optimized for bacterial expression:
(SEQ ID NO: 59)
TTCAATGTACTGATGGTCCATAAACGGAGTCACACTGGCGAGCGCCCGCTCCAAT
GTGAAATCTGCGGGTTCACGTGTCGGCAGAAGGGCAACCTCCTCCGGCATATCA
AGCTGCACACGGGTGAAAAACCGTTTAAGTGCCATCTCTGCAATTACGCCTGTCA
GAGAAGAGATGCTTTG
SD1
Codon-optimized for bacterial expression:
(SEQ ID NO: 60)
TTCAATGTACTGATGGTCCATAAACGGAGTCACACTGGCGAGCGCCCACTCCAAT
GTAAAATCTGCGGGTTCACGTGTCGGCAGAAGGGCAATCTCCTCCGGCATATCA
AGCTGCACACGGGTGAAAAACTGTTTAAGTGCCATCTCTGCAATTACGCCTGTCA
GAGAAGAGATGCTTTG
SD2
Codon-optimized for bacterial expression:
(SEQ ID NO: 61)
TTCAATGTACTGATGGTCCATAAACGGAGTCACACTGGCGAGCGCTCGCTCCAAT
GTAAAATCTGCGGGTTCACGTGTCGGCAGAAGGGCAACCTCCTCCGGCATATCA
AGCTGCACACGGGTGAAAAACTGTTTAAGTGCCATCTCTGCAATTACGCCTGTCA
GAGAAGAGATGCTTTG
SD3
Codon-optimized for bacterial expression:
(SEQ ID NO: 62)
TTCAATGTACTGATGGTCCATAAACGGAGTCACACTGGCGAGCGCTCGCTCCAAT
GTAAAATCTGCGGGTTCACGTGTCGGCAGAAGGGCAACCTCCTCCGGCATATCA
AGCTGCACACGGGTGTAAAACTGTTTAAGTGCCATCTCTGCAATTACGCCTGTCA
GAGAAGAGATGCTTTG
SD4
Codon-optimized for bacterial expression:
(SEQ ID NO: 63)
TTCAATGTACTGCTGGTCCATAAACGGAGTCACACTGGCGAGCGCTCGCTCCAAT
GTAAAATCTGCGGGTTCACGTGTCGGCAGAAGGGCAACCTCCTCCGGCATATCA
AGCTGCACACGGGTGTAAAACTGTTTAAGTGCCATCTCTGCAATTACGCCTGTCA
GAGAAGAGATGCTTTG
SD5
Codon-optimized for bacterial expression:
(SEQ ID NO: 64)
TTCAATGTACTGATGGTCCATAAACGGAGTCACACTGGCGAGCGCCCGTTCCAAT
GTAAAATCTGCGGGTTCACGTGTCGGCAGAAGGGCAAACTCCTCCGGCATATCA
AGCTGCACACGGGTGAAAAACTGTTTAAGTGCCATCTCTGCAATTACGCCTGTCA
GAGAAGAGATGCTTTG
SD6
Codon-optimized for bacterial expression:
(SEQ ID NO: 65)
TTCAATGTACTGATGGTCCATAAACGGAGTCACACTGGCGAGCGCCCGCTCATGT
GTCCTGTGTGCGGGTTCACGTGTCGGCAGAAGGGCAACCTCCTCCGGCATATCAA
GCTGCACATGGGTGAAAAACTGTTTAAGTGCCATCTCTGCAATTACGCCTGTCAG
AGAAGAGATGCTTTG
SD7
Codon-optimized for bacterial expression:
(SEQ ID NO: 66)
TTCAATGTACTGATGGTCCATAAACGGAGTCACACTGGCGAGCGCCCGCTCATGT
GTCCTGTGTGCGGGTTCACGTGTCGGCAGAAGGGCAACCTCCTCCGGCATATCAA
TCTGCACATGGGTGAAAAACTGTTTAAGTGCCATCTCTGCAATTACGCCTGTCAG
AGAAGAGATGCTTTG
SD8
Codon-optimized for mammalian expression:
(SEQ ID NO: 97)
TTCAATGTCCTGATGGTGCATAAAAGATCACATACAGGAGAACGACCGCTGATC
TGTCCCGTATGTGGGTTCACATGCAGACAGAAGGGAAACCTGTTGAGGCATATT
AAATTGCATACCGGGGAAAAACTTTTCAAGTGTCATCTCTGCAATTATGCCTGTC
AGCGGAGGGACGCATTG
Codon-optimized for bacterial expression:
(SEQ ID NO: 67)
TTCAATGTACTGATGGTCCATAAACGGAGTCACACTGGCGAGCGCCCGCTCATTT
GTCCTGTTTGCGGGTTCACGTGTCGGCAGAAGGGCAACCTCCTCCGGCATATCAA
GCTGCACACGGGTGAAAAACTGTTTAAGTGCCATCTCTGCAATTACGCCTGTCAG
AGAAGAGATGCTTTG
SD9
Codon-optimized for bacterial expression:
(SEQ ID NO: 68)
TTCAATGTACTGATGGTCCATAAACGGAGTCACACTGGCGAGCGCCCGCTCATTT
GTCCTGTTTGCGGGTTCACGTGTCGGCAGAAGGGCAACCTCCTCCGGCATATCAA
GCTGCACACGGGTGAAAAACTGTTTAAGTGCCATCTCTGCAAATACGCCTGTCAG
AGAAGAGATGCTTTG
SD10
Codon-optimized for bacterial expression:
(SEQ ID NO: 69)
TTCAATGTACTGATGGTCCATAAACGGAGTCACACTGGCGAGCGCCCGCTCATTT
GTCCTGTTTGCGGGTTCACGTGTCGGCAGAAGGTCAACCTCCTCCGGCATATCAA
GCTGCACACGGGTGAAAAACTGTTTAAGTGCCATCTCTGCAATTACGCCTGTCAG
AGAAGAGATGCTTTG
SD11
Codon-optimized for bacterial expression:
(SEQ ID NO: 70)
TTCAATGTACTGATGGTCCATAAACGGAGTCACACTGGCGAGCGCCCGCTCCAAT
GTGAAATCTGCGGGTTCACGTGTCGGCAGAAGGGCAACCTCCTCCGGCATATCA
AGCTGCACACGGGTGAAAAAACCGTT
SD12
Codon-optimized for bacterial expression:
(SEQ ID NO: 71)
TTCAATGTACTGATGGTCCATAAACGGAGTCACACTGGCGATCGCCTGCTCCAAT
GTAAAATCTGCGGGTTCACGTGTCGGCAGAAGGGCAACCTCCTCCGGCATATCA
AGCTGCACACGGGTGAAAAAACCGTT
SD13
Codon-optimized for bacterial expression:
(SEQ ID NO: 72)
TTCAATGTACTGATGGTCCATAAACGGAGTCACACTGGCGAGCGCCTGCTCCAAT
GTAAAATCTGCGGGTTCACATGTCGGCAGAAGGGCAACCTCCTCCGGCATATCA
AGCTGCACACGGGTGAAAAACCGTTTAAGTGCCATCTCTGCAATTACGCCTGTCA
GAGAAGAGATGCTTTG
SD14
Codon-optimized for bacterial expression:
(SEQ ID NO: 73)
TTCAATGAACTGATGGTCCATAAACGGAGTCACACTGGCGAGCGCCTGCTCCAAT
GTAAAATCTGCGGGTTCACGTGTCGGCAGAAGGGCAACCTCCTCCGGCATATCA
AGCTGCACACGGGTGAAAAACCGTTTAAGTGCCATCTCTGCAATTACGCCTGTCA
GAGAAGAGATGCTTTG
SD15
Codon-optimized for bacterial expression:
(SEQ ID NO: 74)
TTCAATGTACTGATGGTCCATAAACGGAGTCACACTGGCGAGCGCTCGTTCCAAT
GTAGAATCTGCGGGTTCACGTGTCGGCAGAAGGGCAACCTCCTCCGGCACATCA
AGCTGCACACGGGTGTAAAACTGTTTAAGTGCCATCTCTGCAATTACGCCTGTCA
GAGAAGAGATGCTTTG
SD16
Codon-optimized for bacterial expression:
(SEQ ID NO: 75)
TTCAATGTACTGATGGTCCATAAACGGAGTCACCCTGGCGATCGCCTGCTCCAAT
GTAAAATCTGCGGGTTCACGTGTCGGCAGAAGGGCGACCTCCTCCGGCATATCA
AGCTGCACACGGGTGTAAAAACCGTTAAGTGCCATCTCTGCAATTACGCCTGTCA
GAGAAAGAGATGCTTTGTAATC
SD17
Codon-optimized for bacterial expression:
(SEQ ID NO: 76)
TTCAATGTACTGATGGTCCATAAACGGAGTCACACTGGCGATCGCCTGCTCCAAT
GTAAAATCTGCGGGTTCACGTGTCGGCAGAAGGGCAACCTCCTCCGGCATATCA
AGCTGCACACGGGTGTAAAAACCGTTAAGTGCCATCTCTGCAATTACGCCTGTCA
GAGAAAGAGATGCTTTGTAATC
SD18
Codon-optimized for bacterial expression:
(SEQ ID NO: 77)
TTCAATGTACTGATGGGCCATGAACGGAGTCACACTGGCGAGCGCTCGTTCCAAT
GTAGAATCTGCGGGTTCACGTGTCGGCAGAAGGGCAACCTCCTCCGGCACATCA
AGCTGCACACGGATGTAAAACTGCTTAAGTGCCATCTCTGCAATTACGACTGTCA
GAGAAGAGATGCTTTG
SD19
Codon-optimized for bacterial expression:
(SEQ ID NO: 78)
TTCAATGTACTGATGGTCCATGAACGGAGTCACACTGGCGAGCGCTCGTTCCAAT
GTAGAATCTGCGGGTTCACGTGTCGGCAGAAGGGCAACCTCCTCCGGCACATCA
AGCTGCACACGGATGTAAAACTGTTTAAGTGCCATCTCTGCAATTACGCCTGTCA
GAGAAGAGATGCTTTG
SD20
Codon-optimized for bacterial expression:
(SEQ ID NO: 79)
TTCAATGTACTGATGGTCCATAAACGGAGGCACACTGGCGATCGCCTGCTCCAAT
GTAAAATCTGCGGGTTCACGTGTCGGGAGAAGGGCAACCTCCTCCGGCATATCA
AGCTGCACACGGGTGTAAAAACCGTTAAGTGCCATCTCTGCAATTACGCCTGTCA
GAGAAAGAGATGCTTTGTAATC
SD21
Codon-optimized for bacterial expression:
(SEQ ID NO: 80)
TTCAATGTACTGATGGTCCATAAACGGAGTCACACTGGCGATCGCCTGCTCCAAT
GTAAAATCTGCGGGTTCACGTGTCGGGAGAAGGGCAACCTCCTCCGGCATATCA
AGCTGCACACGGGTGTAAAAACCGTTAAGTGCCATCTCTGCAATTACGCCTGTCA
GAGAAAGAGATGCTTTGTAATC
SD22
Codon-optimized for bacterial expression:
(SEQ ID NO: 81)
TTCAATGTACTGATGGTCCATGAACGGAGTCACACTGGCGAGCGCTCGTTCCAAT
GTAGAATCTGCGGGTTCACGTGTCGGCAGAAGGGCAACCTCCTCCGGCACATCA
AGCTGCACACGGATGTAAAACTGTTTAAGTGCCATCTCTGCAATTACGACTGTCA
GAGAAGAGATGCTTTG
SD23
Codon-optimized for mammalian expression:
(SEQ ID NO: 98)
TTCAATGTACTGATGGTCCATAAACGGAGTCACACTGGCGAGCGCAGTTTCCAAT
GTCGGATCTGCGGGTTCACGTGTCGGCAGAAGGGCAACCTCCTCCGGCATATCA
AGCTGCACACGGATGTAAAATTGTTTAAGTGCCATCTCTGCAATTACGCCTGTCA
GAGAAGAGATGCTTTG
SD24
Codon-optimized for mammalian expression:
(SEQ ID NO: 99)
TTCAATGTACTGATGGTCCATAAACGGAGTCACACTGGCGAGCGCCCGTTCCAAT
GTCGGATCTGCGGGTTCACGTGTCGGCAGAAGGGCAACCTCCTCCGGCATATCA
AGCTGCACACGGATGTAAAATTGTTTAAGTGCCATCTCTGCAATTACGCCTGTCA
GAGAAGAGATGCTTTG
SD25
Codon-optimized for mammalian expression:
(SEQ ID NO: 100)
TTCAATGTACTGATGGTCCATAAACGGAGTCACACTGGCGAGCGCCTGCTCCAAT
GTAAGATCTGCGGGTTCACGTGTCGGGAGAAGGGCAACCTCCTCCGGCATATCA
AGCTGCACACGGGTGTAAAAACGGTAAAGTGCCATCTCTGCAATTACGCCTGTC
AGAGAAAGCGGTGTTTCGTA
SD26
Codon-optimized for mammalian expression:
(SEQ ID NO: 101)
TTCAATGTACTGATGGTCCATAAACGGAGACACACTGGCGATCGCCTGCTCCAAT
GTAAGATCTGCGGGTTCACGTGTCGGGAGAAGGGCAACCTCCTCCGGCATATCA
AGCTGCACACGGGTGTAAAAACGGTAAAGTGCCATCTCTGCAATTACGCCTGTC
AGAGAAGAGATGCTTTG
SD27
Codon-optimized for mammalian expression:
(SEQ ID NO: 102)
TTCAATGTACTGATGGTCCATAAACGGAGTCACACTGGCGAGCGCCTGCTCCAAT
GTAAGATCTGCGGGTTCACGTGTCGGGAGAAGGGCAACCTCCTCCGGCATATCA
AGCTGCACACGGGTGTAAAAACGGTAAAGTGCCATCTCTGCAATTACGCCTGTC
AGAGAAGAGATGCTTTG
SD28
Codon-optimized for bacterial expression:
(SEQ ID NO: 82)
TTCAATGTACTGATGGTCCATAAACGGAGTCACACTGGCGAGCGCTCGCTCCATT
GTAAAATCTGCGGGTTCACGTGTCGGCAGAAGGGCAACCTCCTCCGGCATATCA
AGCTGCACACGGGTGTAAAACTGTTTAAGTGCCATCTCTGCAATTACGCCTGTCA
GAGAAGAGATGCTTTG
SD29
Codon-optimized for bacterial expression:
(SEQ ID NO: 83)
TTCAATGTACTGATGGTCTATAAACGGAGTCACACTGGCGAGCGCTCGCTCCATT
GTAAAATCTGCGGGTTCACGTGTCGGCAGAAGGGCAACCTCCTCCGGCATATCA
AGCTGCACACGGGTGTAAAACTGTTTAAGTGCCATCTCTGCAATTACGCCTGTCA
GAGAAGAGATGCTTTG
SD30
Codon-optimized for bacterial expression:
(SEQ ID NO: 84)
TTCAATGTACTGATGGTCTATAAACGGAGTCACACTGGCGAGCGCTCGCTCCACT
GTAAAATCTGCGGGTTCATGTGTCGGCAGAAGGGCAACCTCCTCCGGCATATCA
AGCTGCACACGGGTGTAAAACTGTTTAAGTGCCATCTCTGCAATTACGCCTGTCA
GAGAAGAGATGCTTTG
SD31
Codon-optimized for mammalian expression:
(SEQ ID NO: 103)
TTCAACGTTCTTATGGTGCATAAGCGCAGCCACACAGGAGAGAGGCCACTGTTTT
GCCCAATTTGTGGCTTCACCTGTAGACAAAAGGGAAACCTTCTCCGGCATATCAA
CCTTCACACCGGGGAGAAGTTGTTTAAGTATCATTTGTGCAACTATGCTTGCCAG
CGGCGAGACGCATTG
Codon-optimized for bacterial expression:
(SEQ ID NO: 85)
TTCAATGTACTGATGGTCCATAAACGGAGTCACACTGGCGAGCGCCCGCTCTTTT
GTCCTATTTGCGGGTTCACGTGTCGGCAGAAGGGCAACCTCCTCCGGCATATCAA
TCTGCACACGGGTGAAAAACTGTTTAAGTACCATCTCTGCAATTACGCCTGTCAG
AGAAGAGATGCTTTG
SD32
Codon-optimized for mammalian expression:
(SEQ ID NO: 104)
TTCAATGTACTGATGGTCCATAAACGGAGTCACACTGGCGAGCGCCCGCTCATCT
GTCCGGTCTGCGGGTTCACGTGTCGGCAGAAGGGCAACCTCCTCCGGCATATCAA
TCTGCACCCGGGTGAAAAACTGTTTAAGTACCATCTCTGCAATTACGCCTGTCAG
AGAAGAGATGCTTTG
Codon-optimized for bacterial expression:
(SEQ ID NO: 86)
TTCAATGTACTGATGGTCCATAAACGGAGTCACACTGGCGAGCGCCCGCTCATTT
GTCCTGTTTGCGGGTTCACGTGTCGGCAGAAGGGCAACCTCCTCCGGCATATCAA
TCTGCACCCGGGTGAAAAACTGTTTAAGTACCATCTCTGCAATTACGCCTGTCAG
AGAAGAGATGCTTTG
SD33
Codon-optimized for mammalian expression:
(SEQ ID NO: 105)
TTCAATGTACTGATGGTCCATAAACGGAGTCACACTGGCGAGCGCCTCCTCATCT
GTCCGGTCTGCGGGTTCACGTGTCGGCAGAAGGGCAACCTCCTCCGGCATATCAA
TCTGCACCCGGGTGAAAAACTGTTTAAGTACCATCTCTGCAATTACGCCTGTCAG
AGAAGAGATGCTTTG
Codon-optimized for bacterial expression:
(SEQ ID NO: 87)
TTCAATGTACTGATGGTCCATAAACGGAGTCACACTGGCGAGCGCCTGCTCATTT
GTCCTGTTTGCGGGTTCACGTGTCGGCAGAAGGGCAACCTCCTCCGGCATATCAA
TCTGCACCCGGGTGAAAAACTGTTTAAGTACCATCTCTGCAATTACGCCTGTCAG
AGAAGAGATGCTTTG
SD34
Codon-optimized for bacterial expression:
(SEQ ID NO: 88)
TTCAATGCACTGATGGTCTATAAACGGAGTCACACTGGCGAGCGCTCGCTCCACT
GTAAAATCTGCGGGTTCATGTGTCGGCAGAAGGGCAACCTCCTCCGGCATATCA
AGCTGCACACGGGTGTAAAACTGTTTAAGTGCCATCTCTGCAATTACGCCTGTCA
GAGAAGAAATGCTTTG
SD35
Codon-optimized for mammalian expression:
(SEQ ID NO: 106)
TTCAATGTACTGATGGTCCATAAACGGAGTCACACTGGCGAGCTCCTGCTCTTCT
GTCCGATCTGCGGGTTCACGTGTCGGCAGAAGGGCAACCTCCTCCGGCATATCAA
TCTGCACACGGGTGAAAAACTGTTTAAGTACCATCTCTGCAATTACGCCTGTCAG
AGAAGAGATGCTTTG
Codon-optimized for bacterial expression:
(SEQ ID NO: 89)
TTCAATGTACTGATGGTCCATAAACGGAGTCACACTGGCGAGCTCCTGCTCTTTT
GTCCTATTTGCGGGTTCACGTGTCGGCAGAAGGGCAACCTCCTCCGGCATATCAA
TCTGCACACGGGTGAAAAACTGTTTAAGTACCATCTCTGCAATTACGCCTGTCAG
AGAAGAGATGCTTTG
SD36
Codon-optimized for mammalian expression:
(SEQ ID NO: 107)
TTCAATGTACTGATGGTCCATAAACGGAGTCACACTGGCGAGCTCCTGCTCTTCT
GTCCGATCTGCGGGTTCACGTGTCGGCAGAAGGGCAACCTCCTCCGGCATATCAA
TCTGCACACGGGTGAAAAACTGTTTAAGTACCATCTCTACAATTACGCCTGTCAG
AGAAGAGATGCTTTG
Codon-optimized for bacterial expression:
(SEQ ID NO: 90)
TTCAATGTACTGATGGTCCATAAACGGAGTCACACTGGCGAGCTCCTGCTCTTTT
GTCCTATTTGCGGGTTCACGTGTCGGCAGAAGGGCAACCTCCTCCGGCATATCAA
TCTGCACACGGGTGAAAAACTGTTTAAGTACCATCTCTACAATTACGCCTGTCAG
AGAAGAGATGCTTTG
SD36.2
Codon-optimized for mammalian expression:
(SEQ ID NO: 108)
TTTAACGTGCTCATGGTGCATAAGCGAAGTCATACGGGGGAACGGCTGCTGTTCT
GTCCGATTTGTGGGTTTACGTGCAGGCAAAAAGGTAACTTGCTCCGCCACATAAA
TCTGCACACTGGAGAAAAACTCTTTAAGTATCATCTCTATAACTATGCTTGCCAA
CGAAGGGATGCTTTG
SD36.3
Codon-optimized for mammalian expression:
(SEQ ID NO: 109)
TTCAATGTGCTGATGGTCCATAAACGAAGTCACACAGGTGAGCTGCCGTTGTTTT
GTCCCATTTGCGGCTTTACGTGCCGGCAAAAAGGGAATTTGCTCCGCCACATTAA
CCTTCACACCGGAGAAAAGCTCTTTAAGTACCATCTCTATAATTACGCCTGTCAG
CGCCGCGACGCTTTG
SD36.4
Codon-optimized for mammalian expression:
(SEQ ID NO: 110)
TTTAACGTATTGATGGTTCACAAACGGTCACACACTGGAGAGTTGCTGCTCCAGT
GTCCGATATGTGGCTTCACTTGCCGCCAGAAAGGTAACTTGTTGCGGCATATCAA
TCTCCACACAGGCGAAAAGCTGTTCAAGTACCACCTGTATAACTATGCATGTCAG
AGAAGAGATGCCCTG
SD36.5
Codon-optimized for mammalian expression:
(SEQ ID NO: 111)
TTCAATGTATTGATGGTCCACAAGAGGAGCCACACTGGAGAGCTTTTGCTGTTTT
GCGAAATTTGCGGCTTTACATGCAGACAAAAAGGAAACCTCCTTCGCCACATCA
ACCTTCACACTGGAGAGAAGCTCTTCAAGTACCACTTGTATAACTACGCATGTCA
ACGCCGCGACGCTCTT
SD36.6
Codon-optimized for mammalian expression:
(SEQ ID NO: 112)
TTCAACGTACTCATGGTGCATAAGAGATCACATACGGGTGAGCTCTTGCTCTTTT
GTCCCATTTGCGGTTTTACCTGTCGGCAAAAGGGTAATCTCCTCAGGCACATCAA
GTTGCACACGGGAGAGAAGCTGTTCAAATATCATCTTTACAACTACGCCTGTCAA
AGAAGGGACGCTCTC
SD36.7
Codon-optimized for mammalian expression:
(SEQ ID NO: 113)
TTTAATGTTCTTATGGTCCACAAGAGATCACATACAGGCGAACTCCTGCTGTTTT
GCCCTATTTGCGGATTCACATGCAGGCAGAAGGGAAACCTGCTCCGGCACATCA
ACTTGCATACAGGGGAGAAGCCATTTAAGTACCACCTTTACAACTACGCTTGCCA
ACGCCGGGACGCATTG
SD36.8
Codon-optimized for mammalian expression:
(SEQ ID NO: 114)
TTCAACGTGCTGATGGTCCACAAGCGATCTCACACTGGAGAGCTTCTTCTTTTTTG
CCCTATATGTGGGTTTACTTGCAGACAAAAGGGGAACTTGCTCAGACATATCAAT
CTCCATACAGGAGAAAAACTGTTTAAATGTCACCTCTATAACTATGCGTGTCAGC
GGCGGGATGCACTC
SD36.9
Codon-optimized for mammalian expression:
(SEQ ID NO: (115)
TTCAATGTGCTGATGGTTCACAAACGCAGTCACACTGGGGAACTTTTGCTTTTCT
GCCCTATATGTGGTTTTACATGCCGCCAGAAGGGAAACTTGCTCCGGCATATCAA
TCTCCATACCGGGGAAAAGCTCTTTAAGTGCCACTTGTGTAACTACGCATGTCAG
CGGAGGGACGCGCTG
SD37
Codon-optimized for mammalian expression:
(SEQ ID NO: 116)
CTCCTGTTGTTTTGCCCGATCTGCGGTTTCACTTGTCGGCAGAAAGGCAATCTTCT
CAGGCACATCAACCTCCATACAGGAGAGAAACTCTTCAAGTACCATCTGTATAA
CTATGCCTGTCAACGACGAGATGCCCTG
SD38
Codon-optimized for mammalian expression:
(SEQ ID NO: 117)
TTCAATGTACTGATGGTCCATAAACGGAGTCACACTGGCGAGCTCCTGCTCTTCT
GTCCGATCTGCGGGTTCACGTGTCGGCAGAAGGGCAACCTCCTCCGGCATATCAA
TCTGCACACGGGTGAAAAACTGTTTAAGTACCATCTCTAC
SD39
Codon-optimized for mammalian expression:
(SEQ ID NO: 118)
CTCCTGTTGTTTTGCCCGATCTGCGGTTTCACTTGTCGGCAGAAAGGCAATCTTCT
CAGGCACATCAACCTCCATACAGGAGAGAAACTCTTCAAGTACCATCTGTATAA
CTAT
SD40
Codon-optimized for mammalian expression:
(SEQ ID NO: 119)
CTCCTGCTCTTTTGCCCCATCTGCGGATTCACCTGTAGGCAGAAAGGGAATCTCC
TTCGGCACATCAACTTGCATACAGGTGAGAAATTGTTCAAGTATCATCTGTAC
SD41
Codon-optimized for mammalian expression:
(SEQ ID NO: 120)
CTTCTTCTCTTTTGCCCTATTTGTGGGTTCACTTGCAGACAGAAGGGAAACCTCCT
GCGGCATATCAATCTCCATACCGGAGAGAAACTCTTCAAGTAT
SD42
Codon-optimized for mammalian expression:
(SEQ ID NO: 121)
CTTCTTCTGTTCTGCCCCATCTGTGGGTTTACTTGTAGGCAAAAGGGGAATCTGTT
GCGGCATATCAACCTTCACACTGGAGAGAAACTC
SD43
Codon-optimized for mammalian expression:
(SEQ ID NO: 122)
CTTCTGTTGTTTTGTCCAATCTGCGGGTTCACTTGCCGCCAGAAAGGCAACCTGCT
TCGGCATATAAATTTGCAT
SD44
Codon-optimized for mammalian expression:
(SEQ ID NO: 123)
CTCCTGTTTTGCCCTATTTGTGGATTTACATGCAGGCAAAAAGGTAACCTCCTGAGACATATAAACCTGCAC
SD45
Codon-optimized for bacterial expression:
(SEQ ID NO: 91)
TTCAATGTACTGATGGTCCATAAACGGAGTCACACTGGCGAGCTCCTGCTCTTTT
GTCCTATTTGCGGGTTCACGTGTCGGCAGAAGGGCAACCTCCTCCGGCATATCAA
TCTGCACCCGGGTGAAAAACTGTTTAAGTACCATCTCTACAATTACGCCTGTCAG
AGAAGAGATGCTTTG
SD46
Codon-optimized for bacterial expression:
(SEQ ID NO: 92)
TTCAATGTACTGATGGTCCATAAACGGAGTCACACTGGCGAGCTCCTGCTCTTTT
GTCCTATTTGCGGGTTCACGTGTCGGAAGAAGGGCAACCTCCTCCGGCATATCAA
TCTGCACCCGGGTGAAAAACTGTTTAAGTACCATCTCTACAATTACGCCTGTCAG
AGAAGAGATGCTTTG
SD47
Codon-optimized for bacterial expression:
(SEQ ID NO: 93)
TTCAATGTACTGATGGTCCATAAACGGAGTCACACTGGCGAGCTCCTGCTCTTTT
GTCCTATTTGCGGGTTCACGTGTCGGCAGAAGGGCAACCTCCTCCGGCATATCAA
TCTGCACCCGGGTGAAAAAATGTTTAAGTACCATCTCTACAATTACGCCTATCAG
AGAAGAGATGCTTTG
SD48
Codon-optimized for bacterial expression:
(SEQ ID NO: 94)
TTCAATGTACTGATGGGCCATAAACGGAGTCACACTGGCGATCTCCTGCTCTTTT
GTCCTATTTGCGGGTTCACGTGTCGGCAGGAGGGCAACCTCCTCCGGCATATCAA
TCTGCACCCGGGTGAAAAACTGTTTAAGTACCATCTCCGCAATTACGCCTGTCAG
AGAAGAGATGCTTTG
SD49
Codon-optimized for bacterial expression:
(SEQ ID NO: 95)
CTCAATGTACTGATGGTCCATAGACGGAGTCACACTGGCGAGCTCCTGCTCTTTT
GCCCTATTTGCGGGTTCACGTGTCGGCAGAAGGGCAACCTCCTCCGGCATATCAA
TCTGCACCCGGGTGAAAAACTGTTTAAGTACCATCTCTACCATTACGCCTGTCAG
AGAAGAGATGCTTTG
SD55
Codon-optimized for mammalian expression:
(SEQ ID NO: 126)
TTCAATGTGCTTATGGTCCACAAACGCTCCCACACTGGCGAACTTCTGTTGTTTTG
CCCAATATGCGGTTTCACATGCAGGCAAAAAGGAAATCTGCTGCGCCATATTAA
CCTTCACCCCGGTGAAAAACTCTTCCGGTATCACCTCTATAACTACGCATGTCAA
AGACGAGATGCTCTG
Codon-optimized for bacterial expression:
(SEQ ID NO: 128)
TTCAATGTACTGATGGTCCATAAACGGAGTCACACTGGCGAGCTCCTGCTCTTTT
GTCCTATTTGCGGGTTCACGTGTCGGCAGAAGGGCAACCTCCTCCGGCATATCAA
TCTGCACCCGGGTGAAAAACTGTTTAGGTACCATCTCTACAATTACGCCTGTCAG
AGAAGAGATGCTTTG
SD56
Codon-optimized for mammalian expression:
(SEQ ID NO: 127)
TTCAATGTACTCATGGTACACAAGAGGAGTCATACAGGGGAACTCCTCTTGTTCT
GTCCAATCTGCGGCTTTACTTGTCGCCAAAAAGGTACACTCCTCAGGCACATCAA
CCTTCATCCAGGGGAGAAACTGTTTAGGTATCATCTCTATAACTATGCCTGCCAG
AGGCGGGATGCTCTG
Codon-optimized for bacterial expression:
(SEQ ID NO: 129)
TTCAATGTACTGATGGTCCATAAACGGAGTCACACTGGCGAGCTCCTGCTCTTTT
GTCCTATTTGCGGGTTCACGTGTCGGCAGAAGGGCACCCTCCTCCGGCATATCAA
TCTGCACCCGGGTGAAAAACTGTTTAGGTACCATCTCTACAATTACGCCTGTCAG
AGAAGAGATGCTTTG

What is claimed is:

1. A protein degron comprising an amino acid sequence that is at least 50% identical to the amino acid sequence set forth in SEQ ID NO: 1 and comprises one or more amino acid substitutions at one or more positions recited in Table 1 or Table 2.

2. The protein degron of claim 1 comprising an amino acid sequence that is at least 60%, 65%, 70%, 75%, 80%, 95%, 90%, 95%, or 99% identical to the amino acid sequence set forth in SEQ ID NO: 1.

3. The protein degron of claim 1 or 2 comprising one or more amino acid substitutions at a position selected from F1, V3, M5, V6, H7, K8, S10, T12, E14, R15, P16, L17, Q18, E20, 121, T25, Q28, K29, G30, N31, K37, T40, G41, E42, P44, F45, K46, C47, C50, N51, A53, C54, R57, D58, A59, and L60 relative to SEQ ID NO: 1.

4. The protein degron of any one of claims 1 to 3 comprising 1, 2, 3, 4, 5, 6, 7, 8, 9, 10, 11, 12, 13, 14, or 15 amino acid substitutions relative to SEQ ID NO: 1.

5. The protein degron of any one of claims 1 to 4, wherein the one or more amino acid substitutions are selected from FIL, V3E, V3A, M5L, V6G, H7Y, K8E, K8R, S10R, T12P, E14D, R15L, P16S, P16L, L17F, Q18M, Q18I, Q18H, Q18F, E20K, E20P, E20R, 121V, T25M, Q28E, Q28K, K29E, G30V, N31K, N31D, N31T, K37N, T40M, T40P, G41D, E42V, P44L, P44T, P44M, F45V, F45L, K46R, K46stop, C47Y, C50Y, C50R, N51K, N51H, A53D, C54Y, R57K, D58R, D58N, A59C, and L60F relative to SEQ ID NO: 1.

6. The protein degron of any one of claims 1 to 5 comprising 1, 2, 3, 4, 5, 6, 7, 8, 9, 10, 11, 12, 13, 14, or 15 amino acid substitutions selected from FIL, V3E, V3A, M5L, V6G, H7Y, K8E, K8R, S10R, T12P, E14D, R15L, P16S, P16L, L17F, Q18M, Q18I, Q18H, Q18F, E20K, E20P, E20R, I21V, T25M, Q28E, Q28K, K29E, G30V, N31K, N31D, N31T, K37N, T40M, T40P, G41D, E42V, P44L, P44T, P44M, F45V, F45L, K46R, K46stop, C47Y, C50Y, C50R, N51K, N51H, A53D, C54Y, R57K, D58R, D58N, A59C, and L60F relative to SEQ ID NO: 1.

7. The protein degron of any one of claims 1 to 6 comprising 1, 2, 3, 4, 5, 6, 7, or 8 amino acid substitutions selected from R15L, P16L, Q18F, E20P, K37N, P44L, C47Y, and C50Y relative to SEQ ID NO: 1.

8. The protein degron of any one of claims 1 to 7 comprising the following amino acid substitutions relative to SEQ ID NO: 1: R15L, P16L, Q18F, E20P, K37N, P44L, C47Y, and C50Y.

9. The protein degron of any one of claims 1 to 6 comprising 1, 2, 3, 4, 5, 6, 7, 8, 9, 10, or 11 amino acid substitutions selected from R15L, P16L, Q18F, E20P, N31T, K37N, T40P, P44L, K46R, C47Y, and C50Y relative to SEQ ID NO: 1.

10. The protein degron of any one of claims 1 to 6 and 9 comprising the following amino acid substitutions relative to SEQ ID NO: 1: R15L, P16L, Q18F, E20P, N31T, K37N, T40P, P44L, K46R, C47Y, and C50Y.

11. The protein degron of any one of claims 1 to 10, wherein the protein comprises an amino acid sequence that is at least 70% identical to the amino acid sequence set forth in any one of SEQ ID NOs.: 2-45, 54-58, 124, or 125.

12. The protein degron of any one of claims 1 to 11, wherein the protein comprises an amino acid sequence that is at least 70% sequence identical to the amino acid sequence set forth in SEQ ID NO: 37.

13. The protein degron of any one of claims 1 to 11, wherein the protein comprises an amino acid sequence that is at least 70% sequence identical to the amino acid sequence set forth in SEQ ID NO: 125.

14. The protein degron of any one of claims 1 to 11 comprising the amino acid sequence set forth in any one of SEQ ID NOs: 2-45, 54-58, 124, or 125.

15. The protein degron of any one of claims 1 to 8, 11, and 12 comprising the amino acid sequence set forth in SEQ ID NO: 37.

16. The protein degron of any one of claims 1 to 11 and 13 comprising the amino acid sequence set forth SEQ ID NO: 125.

17. A protein degron comprising an amino acid sequence that is at least 50% identical to amino acid residues 15-50 of SEQ ID NO: 1 and comprises one or more amino acid substitutions at one or more positions selected from R15, P16, Q18, E20, K37, P44, C47, and C50, relative to SEQ ID NO: 1, wherein the protein degron lacks one or more amino acids at one or more of the following ranges of positions: 1-14, 1-15, 40-60, 45-60, 48-60, 51-60, and 53-60, relative to SEQ ID NO: 1.

18. The protein degron of claim 17, wherein the one or more amino acid substitutions are selected from R15L, P16L, Q18F, E20P, K37N, P44L, C47Y, and C50Y relative to SEQ ID NO: 1.

19. The protein degron of claim 17 or 18 comprising the following amino acid substitutions relative to SEQ ID NO: 1: R15L, P16L, Q18F, E20P, K37N, P44L, C47Y, and C50Y.

20. The protein degron of any one of claims 17 to 19, comprising an amino acid sequence that is at least 70% identical to the amino acid sequence set forth in any one of SEQ ID NOs.: 46-53.

21. The protein degron of any one of claims 17 to 20, comprising an amino acid sequence that is at least 70% identical to the amino acid sequence set forth in SEQ ID NO: 49.

22. The protein degron of any one of claims 17 to 21 comprising the amino acid sequence set forth in any one of SEQ ID NOs: 46-53.

23. The protein degron of any one of claims 17 to 22 comprising the amino acid sequence set forth SEQ ID NO: 49.

24. The protein degron of any one of claims 1 to 23, wherein the protein degron binds to cereblon (CRBN) protein in the presence of a small molecule CRBN substrate.

25. The protein degron of claim 24, wherein the small molecule CRBN substrate is VS-777, PT-179, or PK-1016.

26. The protein degron of claim 24 or claim 25, wherein the small molecule CRBN substrate is PT-179, and comprises the structure set forth below:

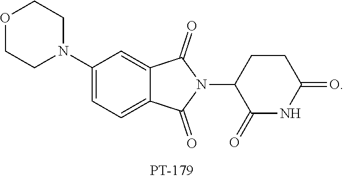 [SMILES: O=C1CCC(N2C(=O)c3ccc(N4CCOCC4)cc3C2=O)C(=O)N1]

27. A nucleic acid sequence that encodes the protein degron of any one of claims 1 to 26.

28. The nucleic acid of claim 23, having at least 70% identity to the nucleic acid sequence set forth in any one of SEQ ID NOs.: 59-123, and 126-129.

29. A vector comprising the nucleic acid of claim 27 or 28.

30. The vector of claim 29, wherein the vector is a phage, plasmid, cosmid, bacmid, or viral vector.

31. The vector of claim 29 or 30, wherein the nucleic acid comprises the sequence set forth in any one of SEQ ID NOs: 59-123 and 126-129.

32. A host cell comprising the protein degron of any one of claims 1-26, the nucleic acid of claim 24 or 25, or the expression vector of any one of claims 29-31.

33. The host cell of claim 32, wherein the host cell is a bacterial cell.

34. The host cell of claim 32 or 33, wherein the host cell is an E. coli cell.

35. A complex comprising a cereblon (CRBN) protein simultaneously bound to a small molecule CRBN substrate and the protein degron of any one of claims 1 to 26.

36. The complex of claim 35, wherein the small molecule is PT-179.

37. The complex of claim 35 or 36 further comprising one or more E3 ubiquitin ligase complex proteins.

38. The complex of claim 37, wherein the one or more E3 ubiquitin ligase complex proteins are selected from damaged DNA binding protein 1 (DDB1), Cullin-4A (CUL4A), and regulator of cullins 1 (ROC1).

39. The complex of any one of claims 35 to 38 further comprising at least one ubiquitin.

40. The complex of any one of claims 35 to 39, wherein the protein degron is connected to a recombinant protein.

41. The complex of claim 40, wherein the recombinant protein is a fusion protein comprising the protein degron and a therapeutic protein.

42. A method of degrading a target protein in a cell, wherein the method comprises contacting a cell comprising cereblon (CRBN), and a target protein having the protein degron of any one of claims 1 to 26, with a small molecule CRBN substrate.

43. The method of claim 42, wherein the target protein is an endogenous protein.

44. The method of claim 42, wherein the target protein is a recombinant protein.

45. The method of any one of claims 42 to 44, wherein the target protein is a therapeutic protein.

46. The method of any one of claims 42 to 45, wherein the cell is in a subject.

47. The method of claim 46, wherein the subject is a mammalian subject.

48. The method of claim 47, wherein the subject is a human.

49. The method of any one of claims 42 to 48, wherein the small molecule CRBN substrate is not thalidomide, lenalidomide, pomalidomide, avadomide, or iberdomide.

50. The method of any one of claims 42 to 49, wherein the small molecule CRBN substrate is VS-777, PT-179, or PK-1016.

51. The method of any one of claims 42 to 50, wherein the small molecule CRBN substrate is PT-179, and has the structure set forth below:

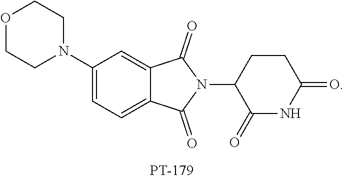 [SMILES: O=C1CCC(N2C(=O)c3ccc(N4CCOCC4)cc3C2=O)C(=O)N1]

52. A method for evolving a protein degron, the method comprising:
(a) contacting a population of bacterial host cells with a population of phages comprising a first nucleic acid encoding a first fusion protein, and deficient in a full-length pIII gene, wherein
(1) the first fusion protein comprises a protein degron of interest and an RNA polymerase subunit,
(2) the population of phages allows for expression of the first fusion protein in the host cells,
(3) the host cells are suitable for phage infection, replication, and packaging; and
(4) the host cells comprise a second nucleic acid encoding full-length pIII protein, and a third nucleic acid sequence encoding a second fusion protein comprising a cereblon (CRBN) and a repressor element, wherein expression of the pIII gene is dependent on interaction of the protein degron of interest of the first fusion protein with the CRBN of the second fusion protein;
(b) incubating the population of host cells and M13 phages under conditions allowing for the modification of the third nucleic acid, the production of infectious M13 phage, and the infection of host cells with M13 phage, wherein infected cells are removed from the population of host cells, and wherein the population of host cells is replenished with fresh host cells that are not infected by M13 phage; and
(c) isolating a modified M13 phage replication product encoding an evolved variant of the first fusion protein from the population of host cells.

53. The method of claim 52, wherein the RNA polymerase subunit is RNA polymerase omega (RpoZ) subunit.

54. The method of any one of claims 52 to 53, wherein the bacterial host cells are E. coli cells.

55. The method of any one of claims 52 to 54, wherein the phages are filamentous phages.

56. The method of any one of claims 52 to 55, wherein the phages are M13 phages.

57. The method of any one of claims 52 to 56, comprising incubating the population of host cells and M13 phages with a small molecule CRBN substrate.

58. The method of claim 57, wherein the small molecule CRBN substrate is PT-179, and has the structure set forth below:

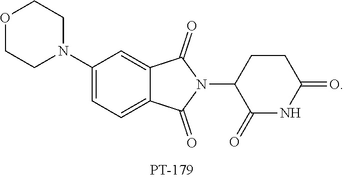 [SMILES: O=C1CCC(N2C(=O)c3ccc(N4CCOCC4)cc3C2=O)C(=O)N1]

59. The method of any one of claims 52 to 58, wherein the protein degron of interest comprises the amino acid sequence set forth in SEQ ID NO: 1.

60. The method of any of one of claims 52 to 59, wherein the host cells further comprise a helper plasmid and/or a mutagenesis plasmid.

61. The method of any one of claims 52 to 60, wherein the second nucleic acid encoding full-length pIII protein further comprises a promoter.

62. The method of claim 61, wherein the promoter is a lacZ promoter or a mutant lacZ promoter.

63. The method of any one of claims 52 to 62, wherein the expression construct encoding full-length pIII protein further comprises a repressor binding site.

64. The method of claim 63, wherein the repressor binding site comprises an RR69 repressor binding site.

65. The method of claim 63, wherein the repressor binding site comprises an sc-p22cI repressor binding site.

66. A vector system comprising:
(i) a first nucleic acid encoding a fusion protein comprising a protein degron of interest and an RNA polymerase subunit;
(ii) a second nucleic acid encoding a full-length pIII protein; and
(iii) a third nucleic acid encoding a fusion protein comprising cereblon (CRBN) and a phage repressor,
wherein the nucleic acid sequence encoding the full-length pIII protein is under the control of an conditional promoter, and comprises one or more phage repressor binding sites.

67. The vector system of claim 66, wherein the protein degron of interest comprises the amino acid sequence set forth in SEQ ID NO: 1.

68. The vector system of claim 66 or 67, wherein the phage repressor comprises an RR69 phage repressor

69. The vector system of claim 66 or 67, wherein the phage repressor comprises a single chain p22 phage repressor (sc-p22cI).

70. The vector system of any one of claims 66 to 69, wherein the conditional promoter comprises a LacZ promoter or a mutant lacZ promoter.

71. The vector system of any one of claims 66 to 70, wherein each of the first nucleic acid, second nucleic acid, and third nucleic acid are on a separate vector.

72. The vector system of claim 71, wherein each separate vector is independently selected from a phage vector or plasmid.

73. The vector system of any one of claims 66 to 72 further comprising a mutagenesis plasmid.

74. The vector system of claim 73, wherein the mutagenesis plasmid comprises an arabinose-inducible promoter.

75. A fusion protein comprising the protein degron of any one of claims 1 to 26 and a target protein.

76. The fusion protein of claim 75, wherein the target protein is an endogenous protein.

77. The fusion protein of claim 75 or 76, wherein the target protein is a recombinant protein.

78. The fusion protein of any one of claims 75 to 77, wherein the target protein is a therapeutic protein.


SEQUENCE LISTING (135 sequences)

SEQ ID NO: 1 - protein, 60 aa, Synthetic construct
FNVLMVHKRSHTGERPLQCEICGFTCRQKGNLLRHIKLHTGEKPFKCHLCNYACQRRDAL

SEQ ID NO: 2 - protein, 60 aa, Synthetic construct
FNVLMVHKRSHTGERPLQCKICGFTCRQKGNLLRHIKLHTGEKLFKCHLCNYACQRRDAL

SEQ ID NO: 3 - protein, 60 aa, Synthetic construct
FNVLMVHKRSHTGERSLQCKICGFTCRQKGNLLRHIKLHTGEKLFKCHLCNYACQRRDAL

SEQ ID NO: 4 - protein, 60 aa, Synthetic construct
FNVLMVHKRSHTGERSLQCKICGFTCRQKGNLLRHIKLHTGVKLFKCHLCNYACQRRDAL

SEQ ID NO: 5 - protein, 60 aa, Synthetic construct
FNVLLVHKRSHTGERSLQCKICGFTCRQKGNLLRHIKLHTGVKLFKCHLCNYACQRRDAL

SEQ ID NO: 6 - protein, 60 aa, Synthetic construct
FNVLMVHKRSHTGERPFQCKICGFTCRQKGKLLRHIKLHTGEKLFKCHLCNYACQRRDAL

SEQ ID NO: 7 - protein, 60 aa, Synthetic construct
FNVLMVHKRSHTGERPLMCPVCGFTCRQKGNLLRHIKLHMGEKLFKCHLCNYACQRRDAL

SEQ ID NO: 8 - protein, 60 aa, Synthetic construct
FNVLMVHKRSHTGERPLMCPVCGFTCRQKGKLLRHIKLHMGEKLFKCHLCNYACQRRDAL

SEQ ID NO: 9 - protein, 60 aa, Synthetic construct
FNVLMVHKRSHTGERPLICPVCGFTCRQKGNLLRHIKLHTGEKLFKCHLCNYACQRRDAL

SEQ ID NO: 10 - protein, 60 aa, Synthetic construct
FNVLMVHKRSHTGERPLICPVCGFTCRQKGNLLRHIKLHTGEKLFKCHLCKYACQRRDAL

SEQ ID NO: 11 - protein, 60 aa, Synthetic construct
FNVLMVHKRSHTGERPLICPVCGFTCRQKVNLLRHIKLHTGEKLFKCHLCNYACQRRDAL

SEQ ID NO: 12 - protein, 45 aa, Synthetic construct
FNVLMVHKRSHTGERPLQCEICGFTCRQKGNLLRHIKLHTGEKTV

SEQ ID NO: 13 - protein, 45 aa, Synthetic construct
FNVLMVHKRSHTGDRLLQCKICGFTCRQKGNLLRHIKLHTGEKTV

SEQ ID NO: 14 - protein, 60 aa, Synthetic construct
FNVLMVHKRSHTGERLLQCKICGFTCRQKGNLLRHIKLHTGEKPFKCHLCNYACQRRDAL

SEQ ID NO: 15 - protein, 60 aa, Synthetic construct
FNELMVHKRSHTGERLLQCKICGFTCRQKGNLLRHIKLHTGEKPFKCHLCNYACQRRDAL

SEQ ID NO: 16 - protein, 60 aa, Synthetic construct
FNVLMVHKRSHTGERSFQCRICGFTCRQKGNLLRHIKLHTGVKLFKCHLCNYACQRRDAL

SEQ ID NO: 17 - protein, 62 aa, Synthetic construct
FNVLMVHKRSHPGDRLLQCKICGFTCRQKGDLLRHIKLHTGVKTVKCHLCNYACQRKRCFVI

SEQ ID NO: 18 - protein, 62 aa, Synthetic construct
FNVLMVHKRSHTGDRLLQCKICGFTCRQKGNLLRHIKLHTGVKTVKCHLCNYACQRKRCFVI

SEQ ID NO: 19 - protein, 60 aa, Synthetic construct
FNVLMGHERSHTGERSFQCRICGFTCRQKGNLLRHIKLHTDVKLLKCHLCNYDCQRRDAL

SEQ ID NO: 20 - protein, 60 aa, Synthetic construct
FNVLMVHERSHTGERSFQCRICGFTCRQKGNLLRHIKLHTDVKLFKCHLCNYACQRRDAL

SEQ ID NO: 21 - protein, 62 aa, Synthetic construct
FNVLMVHKRRHTGDRLLQCKICGFTCREKGNLLRHIKLHTGVKTVKCHLCNYACQRKRCFVI

SEQ ID NO: 22 - protein, 62 aa, Synthetic construct
FNVLMVHKRSHTGDRLLQCKICGFTCREKGNLLRHIKLHTGVKTVKCHLCNYACQRKRCFVI

SEQ ID NO: 23 - protein, 60 aa, Synthetic construct
FNVLMVHERSHTGERSFQCRICGFTCRQKGNLLRHIKLHTDVKLFKCHLCNYDCQRRDAL

SEQ ID NO: 24 - protein, 60 aa, Synthetic construct
FNVLMVHKRSHTGERSFQCRICGFTCRQKGNLLRHIKLHTDVKLFKCHLCNYACQRRDAL

SEQ ID NO: 25 - protein, 60 aa, Synthetic construct
FNVLMVHKRSHTGERPFQCRICGFTCRQKGNLLRHIKLHTDVKLFKCHLCNYACQRRDAL

SEQ ID NO: 26 - protein, 62 aa, Synthetic construct
FNVLMVHKRSHTGERLLQCKICGFTCREKGNLLRHIKLHTGVKTVKCHLCNYACQRKRCFVI

SEQ ID NO: 27 - protein, 60 aa, Synthetic construct
FNVLMVHKRRHTGDRLLQCKICGFTCREKGNLLRHIKLHTGVKTVKCHLCNYACQRRDAL

SEQ ID NO: 28 - protein, 60 aa, Synthetic construct
FNVLMVHKRSHTGERLLQCKICGFTCREKGNLLRHIKLHTGVKTVKCHLCNYACQRRDAL

SEQ ID NO: 29 - protein, 60 aa, Synthetic construct
FNVLMVHKRSHTGERSLHCKICGFTCRQKGNLLRHIKLHTGVKLFKCHLCNYACQRRDAL

SEQ ID NO: 30 - protein, 60 aa, Synthetic construct
FNVLMVYKRSHTGERSLHCKICGFTCRQKGNLLRHIKLHTGVKLFKCHLCNYACQRRDAL

SEQ ID NO: 31 - protein, 60 aa, Synthetic construct
FNVLMVYKRSHTGERSLHCKICGFMCRQKGNLLRHIKLHTGVKLFKCHLCNYACQRRDAL

SEQ ID NO: 32 - protein, 60 aa, Synthetic construct
FNVLMVHKRSHTGERPLFCPICGFTCRQKGNLLRHINLHTGEKLFKYHLCNYACQRRDAL

SEQ ID NO: 33 - protein, 60 aa, Synthetic construct
FNVLMVHKRSHTGERPLICPVCGFTCRQKGNLLRHINLHPGEKLFKYHLCNYACQRRDAL

SEQ ID NO: 34 - protein, 60 aa, Synthetic construct
FNVLMVHKRSHTGERLLICPVCGFTCRQKGNLLRHINLHPGEKLFKYHLCNYACQRRDAL

SEQ ID NO: 35 - protein, 60 aa, Synthetic construct
FNALMVYKRSHTGERSLHCKICGFMCRQKGNLLRHIKLHTGVKLFKCHLCNYACQRRNAL

SEQ ID NO: 36 - protein, 60 aa, Synthetic construct
FNVLMVHKRSHTGELLLFCPICGFTCRQKGNLLRHINLHTGEKLFKYHLCNYACQRRDAL

SEQ ID NO: 37 - protein, 60 aa, Synthetic construct
FNVLMVHKRSHTGELLLFCPICGFTCRQKGNLLRHINLHTGEKLFKYHLYNYACQRRDAL

SEQ ID NO: 38 - protein, 60 aa, Synthetic construct
FNVLMVHKRSHTGERLLFCPICGFTCRQKGNLLRHINLHTGEKLFKYHLYNYACQRRDAL

SEQ ID NO: 39 - protein, 60 aa, Synthetic construct
FNVLMVHKRSHTGELPLFCPICGFTCRQKGNLLRHINLHTGEKLFKYHLYNYACQRRDAL

SEQ ID NO: 40 - protein, 60 aa, Synthetic construct
FNVLMVHKRSHTGELLLQCPICGFTCRQKGNLLRHINLHTGEKLFKYHLYNYACQRRDAL

SEQ ID NO: 41 - protein, 60 aa, Synthetic construct
FNVLMVHKRSHTGELLLFCEICGFTCRQKGNLLRHINLHTGEKLFKYHLYNYACQRRDAL

SEQ ID NO: 42 - protein, 60 aa, Synthetic construct
FNVLMVHKRSHTGELLLFCPICGFTCRQKGNLLRHIKLHTGEKLFKYHLYNYACQRRDAL

SEQ ID NO: 43 - protein, 60 aa, Synthetic construct
FNVLMVHKRSHTGELLLFCPICGFTCRQKGNLLRHINLHTGEKPFKYHLYNYACQRRDAL

SEQ ID NO: 44 - protein, 60 aa, Synthetic construct
FNVLMVHKRSHTGELLLFCPICGFTCRQKGNLLRHINLHTGEKLFKCHLYNYACQRRDAL

SEQ ID NO: 45 - protein, 60 aa, Synthetic construct; identical to SEQ ID NO: 36

SEQ ID NO: 46 - protein, 46 aa, Synthetic construct
LLLFCPICGFTCRQKGNLLRHINLHTGEKLFKYHLYNYACQRRDAL

SEQ ID NO: 47 - protein, 50 aa, Synthetic construct
FNVLMVHKRSHTGELLLFCPICGFTCRQKGNLLRHINLHTGEKLFKYHLY

SEQ ID NO: 48 - protein, 38 aa, Synthetic construct
LLLFCPICGFTCRQKGNLLRHINLHTGEKLFKYHLYNY

SEQ ID NO: 49 - protein, 36 aa, Synthetic construct
LLLFCPICGFTCRQKGNLLRHINLHTGEKLFKYHLY

SEQ ID NO: 50 - protein, 33 aa, Synthetic construct
LLLFCPICGFTCRQKGNLLRHINLHTGEKLFKY

SEQ ID NO: 51 - protein, 30 aa, Synthetic construct
LLLFCPICGFTCRQKGNLLRHINLHTGEKL

SEQ ID NO: 52 - protein, 25 aa, Synthetic construct
LLLFCPICGFTCRQKGNLLRHINLH

SEQ ID NO: 53 - protein, 24 aa, Synthetic construct
LLFCPICGFTCRQKGNLLRHINLH

SEQ ID NO: 54 - protein, 60 aa, Synthetic construct
FNVLMVHKRSHTGELLLFCPICGFTCRQKGNLLRHINLHPGEKLFKYHLYNYACQRRDAL

SEQ ID NO: 55 - protein, 60 aa, Synthetic construct
FNVLMVHKRSHTGELLLFCPICGFTCRKKGNLLRHINLHPGEKLFKYHLYNYACQRRDAL

SEQ ID NO: 56 - protein, 60 aa, Synthetic construct
FNVLMVHKRSHTGELLLFCPICGFTCRQKGNLLRHINLHPGEKMFKYHLYNYAYQRRDAL

SEQ ID NO: 57 - protein, 60 aa, Synthetic construct
FNVLMGHKRSHTGDLLLFCPICGFTCRQEGNLLRHINLHPGEKLFKYHLRNYACQRRDAL

SEQ ID NO: 58 - protein, 60 aa, Synthetic construct
LNVLMVHRRSHTGELLLFCPICGFTCRQKGNLLRHINLHPGEKLFKYHLYHYACQRRDAL

SEQ ID NO: 59 - DNA, 180 bp, Synthetic construct
  source 1..180: mol_type = other DNA
ttcaatgtactgatggtccataaacggagtcacactggcgagcgcccgctccaatgtgaaatctgcgggttcacgtgtcggcagaagggcaacctcctccggcatatcaagctgcacacgggtgaaaaaccgtttaagtgccatctctgcaattacgcctgtcagagaagagatgctttg

SEQ ID NO: 60 - DNA, 180 bp, Synthetic construct
  source 1..180: mol_type = other DNA
ttcaatgtactgatggtccataaacggagtcacactggcgagcgcccactccaatgtaaaatctgcgggttcacgtgtcggcagaagggcaatctcctccggcatatcaagctgcacacgggtgaaaaactgtttaagtgccatctctgcaattacgcctgtcagagaagagatgctttg

SEQ ID NO: 61 - DNA, 180 bp, Synthetic construct
  source 1..180: mol_type = other DNA
ttcaatgtactgatggtccataaacggagtcacactggcgagcgctcgctccaatgtaaaatctgcgggttcacgtgtcggcagaagggcaacctcctccggcatatcaagctgcacacgggtgaaaaactgtttaagtgccatctctgcaattacgcctgtcagagaagagatgctttg

SEQ ID NO: 62 - DNA, 180 bp, Synthetic construct
  source 1..180: mol_type = other DNA
ttcaatgtactgatggtccataaacggagtcacactggcgagcgctcgctccaatgtaaaatctgcgggttcacgtgtcggcagaagggcaacctcctccggcatatcaagctgcacacgggtgtaaaactgtttaagtgccatctctgcaattacgcctgtcagagaagagatgctttg

SEQ ID NO: 63 - DNA, 180 bp, Synthetic construct
  source 1..180: mol_type = other DNA
ttcaatgtactgctggtccataaacggagtcacactggcgagcgctcgctccaatgtaaaatctgcgggttcacgtgtcggcagaagggcaacctcctccggcatatcaagctgcacacgggtgtaaaactgtttaagtgccatctctgcaattacgcctgtcagagaagagatgctttg

SEQ ID NO: 64 - DNA, 180 bp, Synthetic construct
  source 1..180: mol_type = other DNA
ttcaatgtactgatggtccataaacggagtcacactggcgagcgcccgttccaatgtaaaatctgcgggttcacgtgtcggcagaagggcaaactcctccggcatatcaagctgcacacgggtgaaaaactgtttaagtgccatctctgcaattacgcctgtcagagaagagatgctttg

SEQ ID NO: 65 - DNA, 180 bp, Synthetic construct
  source 1..180: mol_type = other DNA
ttcaatgtactgatggtccataaacggagtcacactggcgagcgcccgctcatgtgtcctgtgtgcgggttcacgtgtcggcagaagggcaacctcctccggcatatcaagctgcacatgggtgaaaaactgtttaagtgccatctctgcaattacgcctgtcagagaagagatgctttg

SEQ ID NO: 66 - DNA, 180 bp, Synthetic construct
  source 1..180: mol_type = other DNA
ttcaatgtactgatggtccataaacggagtcacactggcgagcgcccgctcatgtgtcctgtgtgcgggttcacgtgtcggcagaagggcaacctcctccggcatatcaatctgcacatgggtgaaaaactgtttaagtgccatctctgcaattacgcctgtcagagaagagatgctttg

SEQ ID NO: 67 - DNA, 180 bp, Synthetic construct
  source 1..180: mol_type = other DNA
ttcaatgtactgatggtccataaacggagtcacactggcgagcgcccgctcatttgtcctgtttgcgggttcacgtgtcggcagaagggcaacctcctccggcatatcaagctgcacacgggtgaaaaactgtttaagtgccatctctgcaattacgcctgtcagagaagagatgctttg

SEQ ID NO: 68 - DNA, 180 bp, Synthetic construct
  source 1..180: mol_type = other DNA
ttcaatgtactgatggtccataaacggagtcacactggcgagcgcccgctcatttgtcctgtttgcgggttcacgtgtcggcagaagggcaacctcctccggcatatcaagctgcacacgggtgaaaaactgtttaagtgccatctctgcaaatacgcctgtcagagaagagatgctttg

SEQ ID NO: 69 - DNA, 180 bp, Synthetic construct
  source 1..180: mol_type = other DNA
ttcaatgtactgatggtccataaacggagtcacactggcgagcgcccgctcatttgtcctgtttgcgggttcacgtgtcggcagaaggtcaacctcctccggcatatcaagctgcacacgggtgaaaaactgtttaagtgccatctctgcaattacgcctgtcagagaagagatgctttg

SEQ ID NO: 70 - DNA, 135 bp, Synthetic construct
  source 1..135: mol_type = other DNA
ttcaatgtactgatggtccataaacggagtcacactggcgagcgcccgctccaatgtgaaatctgcgggttcacgtgtcggcagaagggcaacctcctccggcatatcaagctgcacacgggtgaaaaaaccgtt

SEQ ID NO: 71 - DNA, 135 bp, Synthetic construct
  source 1..135: mol_type = other DNA
ttcaatgtactgatggtccataaacggagtcacactggcgatcgcctgctccaatgtaaaatctgcgggttcacgtgtcggcagaagggcaacctcctccggcatatcaagctgcacacgggtgaaaaaaccgtt

SEQ ID NO: 72 - DNA, 180 bp, Synthetic construct
  source 1..180: mol_type = other DNA
ttcaatgtactgatggtccataaacggagtcacactggcgagcgcctgctccaatgtaaaatctgcgggttcacatgtcggcagaagggcaacctcctccggcatatcaagctgcacacgggtgaaaaaccgtttaagtgccatctctgcaattacgcctgtcagagaagagatgctttg

SEQ ID NO: 73 - DNA, 180 bp, Synthetic construct
  source 1..180: mol_type = other DNA
ttcaatgaactgatggtccataaacggagtcacactggcgagcgcctgctccaatgtaaaatctgcgggttcacgtgtcggcagaagggcaacctcctccggcatatcaagctgcacacgggtgaaaaaccgtttaagtgccatctctgcaattacgcctgtcagagaagagatgctttg

SEQ ID NO: 74 - DNA, 180 bp, Synthetic construct
  source 1..180: mol_type = other DNA
ttcaatgtactgatggtccataaacggagtcacactggcgagcgctcgttccaatgtagaatctgcgggttcacgtgtcggcagaagggcaacctcctccggcacatcaagctgcacacgggtgtaaaactgtttaagtgccatctctgcaattacgcctgtcagagaagagatgctttg

SEQ ID NO: 75 - DNA, 186 bp, Synthetic construct
  source 1..186: mol_type = other DNA
ttcaatgtactgatggtccataaacggagtcaccctggcgatcgcctgctccaatgtaaaatctgcgggttcacgtgtcggcagaagggcgacctcctccggcatatcaagctgcacacgggtgtaaaaaccgttaagtgccatctctgcaattacgcctgtcagagaaagagatgctttgtaatc

SEQ ID NO: 76 - DNA, 186 bp, Synthetic construct
  source 1..186: mol_type = other DNA
ttcaatgtactgatggtccataaacggagtcacactggcgatcgcctgctccaatgtaaaatctgcgggttcacgtgtcggcagaagggcaacctcctccggcatatcaagctgcacacgggtgtaaaaaccgttaagtgccatctctgcaattacgcctgtcagagaaagagatgctttgtaatc

SEQ ID NO: 77 - DNA, 180 bp, Synthetic construct
  source 1..180: mol_type = other DNA
ttcaatgtactgatgggccatgaacggagtcacactggcgagcgctcgttccaatgtagaatctgcgggttcacgtgtcggcagaagggcaacctcctccggcacatcaagctgcacacggatgtaaaactgcttaagtgccatctctgcaattacgactgtcagagaagagatgctttg

SEQ ID NO: 78 - DNA, 180 bp, Synthetic construct
  source 1..180: mol_type = other DNA
ttcaatgtactgatggtccatgaacggagtcacactggcgagcgctcgttccaatgtagaatctgcgggttcacgtgtcggcagaagggcaacctcctccggcacatcaagctgcacacggatgtaaaactgtttaagtgccatctctgcaattacgcctgtcagagaagagatgctttg

SEQ ID NO: 79 - DNA, 186 bp, Synthetic construct
  source 1..186: mol_type = other DNA
ttcaatgtactgatggtccataaacggaggcacactggcgatcgcctgctccaatgtaaaatctgcgggttcacgtgtcgggagaagggcaacctcctccggcatatcaagctgcacacgggtgtaaaaaccgttaagtgccatctctgcaattacgcctgtcagagaaagagatgctttgtaatc

SEQ ID NO: 80 - DNA, 186 bp, Synthetic construct
  source 1..186: mol_type = other DNA
ttcaatgtactgatggtccataaacggagtcacactggcgatcgcctgctccaatgtaaaatctgcgggttcacgtgtcgggagaagggcaacctcctccggcatatcaagctgcacacgggtgtaaaaaccgttaagtgccatctctgcaattacgcctgtcagagaaagagatgctttgtaatc

SEQ ID NO: 81 - DNA, 180 bp, Synthetic construct
  source 1..180: mol_type = other DNA
ttcaatgtactgatggtccatgaacggagtcacactggcgagcgctcgttccaatgtagaatctgcgggttcacgtgtcggcagaagggcaacctcctccggcacatcaagctgcacacggatgtaaaactgtttaagtgccatctctgcaattacgactgtcagagaagagatgctttg

SEQ ID NO: 82 - DNA, 180 bp, Synthetic construct
  source 1..180: mol_type = other DNA
ttcaatgtactgatggtccataaacggagtcacactggcgagcgctcgctccattgtaaaatctgcgggttcacgtgtcggcagaagggcaacctcctccggcatatcaagctgcacacgggtgtaaaactgtttaagtgccatctctgcaattacgcctgtcagagaagagatgctttg

SEQ ID NO: 83 - DNA, 180 bp, Synthetic construct
  source 1..180: mol_type = other DNA
ttcaatgtactgatggtctataaacggagtcacactggcgagcgctcgctccattgtaaaatctgcgggttcacgtgtcggcagaagggcaacctcctccggcatatcaagctgcacacgggtgtaaaactgtttaagtgccatctctgcaattacgcctgtcagagaagagatgctttg

SEQ ID NO: 84 - DNA, 180 bp, Synthetic construct
  source 1..180: mol_type = other DNA
ttcaatgtactgatggtctataaacggagtcacactggcgagcgctcgctccactgtaaaatctgcgggttcatgtgtcggcagaagggcaacctcctccggcatatcaagctgcacacgggtgtaaaactgtttaagtgccatctctgcaattacgcctgtcagagaagagatgctttg

SEQ ID NO: 85 - DNA, 180 bp, Synthetic construct
  source 1..180: mol_type = other DNA
ttcaatgtactgatggtccataaacggagtcacactggcgagcgcccgctcttttgtcctatttgcgggttcacgtgtcggcagaagggcaacctcctccggcatatcaatctgcacacgggtgaaaaactgtttaagtaccatctctgcaattacgcctgtcagagaagagatgctttg

SEQ ID NO: 86 - DNA, 180 bp, Synthetic construct
  source 1..180: mol_type = other DNA
ttcaatgtactgatggtccataaacggagtcacactggcgagcgcccgctcatttgtcctgtttgcgggttcacgtgtcggcagaagggcaacctcctccggcatatcaatctgcacccgggtgaaaaactgtttaagtaccatctctgcaattacgcctgtcagagaagagatgctttg

SEQ ID NO: 87 - DNA, 180 bp, Synthetic construct
  source 1..180: mol_type = other DNA
ttcaatgtactgatggtccataaacggagtcacactggcgagcgcctgctcatttgtcctgtttgcgggttcacgtgtcggcagaagggcaacctcctccggcatatcaatctgcacccgggtgaaaaactgtttaagtaccatctctgcaattacgcctgtcagagaagagatgctttg

SEQ ID NO: 88 - DNA, 180 bp, Synthetic construct
  source 1..180: mol_type = other DNA
ttcaatgcactgatggtctataaacggagtcacactggcgagcgctcgctccactgtaaaatctgcgggttcatgtgtcggcagaagggcaacctcctccggcatatcaagctgcacacgggtgtaaaactgtttaagtgccatctctgcaattacgcctgtcagagaagaaatgctttg

SEQ ID NO: 89 - DNA, 180 bp, Synthetic construct
  source 1..180: mol_type = other DNA
ttcaatgtactgatggtccataaacggagtcacactggcgagctcctgctcttttgtcctatttgcgggttcacgtgtcggcagaagggcaacctcctccggcatatcaatctgcacacgggtgaaaaactgtttaagtaccatctctgcaattacgcctgtcagagaagagatgctttg

SEQ ID NO: 90 - DNA, 180 bp, Synthetic construct
  source 1..180: mol_type = other DNA
ttcaatgtactgatggtccataaacggagtcacactggcgagctcctgctcttttgtcctatttgcgggttcacgtgtcggcagaagggcaacctcctccggcatatcaatctgcacacgggtgaaaaactgtttaagtaccatctctacaattacgcctgtcagagaagagatgctttg

SEQ ID NO: 91 - DNA, 180 bp, synthetic construct
  source 1..180: mol_type = other DNA
ttcaatgtactgatggtccataaacggagtcacactggcgagctcctgctcttttgtcctatttgcgggttcacgtgtcggcagaagggcaacctcctccggcatatcaatctgcacccgggtgaaaaactgtttaagtaccatctctacaattacgcctgtcagagaagagatgctttg

SEQ ID NO: 92 - DNA, 180 bp, synthetic construct
  source 1..180: mol_type = other DNA
ttcaatgtactgatggtccataaacggagtcacactggcgagctcctgctcttttgtcctatttgcgggttcacgtgtcggaagaagggcaacctcctccggcatatcaatctgcacccgggtgaaaaactgtttaagtaccatctctacaattacgcctgtcagagaagagatgctttg

SEQ ID NO: 93 - DNA, 180 bp, synthetic construct
  source 1..180: mol_type = other DNA
ttcaatgtactgatggtccataaacggagtcacactggcgagctcctgctcttttgtcctatttgcgggttcacgtgtcggcagaagggcaacctcctccggcatatcaatctgcacccgggtgaaaaaatgtttaagtaccatctctacaattacgcctatcagagaagagatgctttg

SEQ ID NO: 94 - DNA, 180 bp, synthetic construct
  source 1..180: mol_type = other DNA
ttcaatgtactgatgggccataaacggagtcacactggcgatctcctgctcttttgtcctatttgcgggttcacgtgtcggcaggagggcaacctcctccggcatatcaatctgcacccgggtgaaaaactgtttaagtaccatctccgcaattacgcctgtcagagaagagatgctttg

SEQ ID NO: 95 - DNA, 180 bp, synthetic construct
  source 1..180: mol_type = other DNA
ctcaatgtactgatggtccatagacggagtcacactggcgagctcctgctcttttgccctatttgcgggttcacgtgtcggcagaagggcaacctcctccggcatatcaatctgcacccgggtgaaaaactgtttaagtaccatctctaccattacgcctgtcagagaagagatgctttg

SEQ ID NO: 96 - DNA, 180 bp, Synthetic construct
  source 1..180: mol_type = other DNA
ttcaatgttctgatggttcataaacggtcccacaccggcgaaaggcccttgcaatgtgagatatgcggcttcacttgtcgccaaaaaggtaacctgcttagacacatcaaacttcatactggcgagaaacctttcaaatgtcatttgtgcaattatgcctgtcaaagacgcgacgcactg

SEQ ID NO: 97 - DNA, 180 bp, Synthetic construct
  source 1..180: mol_type = other DNA
ttcaatgtcctgatggtgcataaaagatcacatacaggagaacgaccgctgatctgtcccgtatgtgggttcacatgcagacagaagggaaacctgttgaggcatattaaattgcataccggggaaaaacttttcaagtgtcatctctgcaattatgcctgtcagcggagggacgcattg

SEQ ID NO: 98 - DNA, 180 bp, Synthetic construct
  source 1..180: mol_type = other DNA
ttcaatgtactgatggtccataaacggagtcacactggcgagcgcagtttccaatgtcggatctgcgggttcacgtgtcggcagaagggcaacctcctccggcatatcaagctgcacacggatgtaaaattgtttaagtgccatctctgcaattacgcctgtcagagaagagatgctttg

SEQ ID NO: 99 - DNA, 180 bp, Synthetic construct
  source 1..180: mol_type = other DNA
ttcaatgtactgatggtccataaacggagtcacactggcgagcgcccgttccaatgtcggatctgcgggttcacgtgtcggcagaagggcaacctcctccggcatatcaagctgcacacggatgtaaaattgtttaagtgccatctctgcaattacgcctgtcagagaagagatgctttg

SEQ ID NO: 100 - DNA, 183 bp, Synthetic construct
  source 1..183: mol_type = other DNA
ttcaatgtactgatggtccataaacggagtcacactggcgagcgcctgctccaatgtaagatctgcgggttcacgtgtcgggagaagggcaacctcctccggcatatcaagctgcacacgggtgtaaaaacggtaaagtgccatctctgcaattacgcctgtcagagaaagcggtgtttcgta

SEQ ID NO: 101 - DNA, 180 bp, Synthetic construct
  source 1..180: mol_type = other DNA
ttcaatgtactgatggtccataaacggagacacactggcgatcgcctgctccaatgtaagatctgcgggttcacgtgtcgggagaagggcaacctcctccggcatatcaagctgcacacgggtgtaaaaacggtaaagtgccatctctgcaattacgcctgtcagagaagagatgctttg

SEQ ID NO: 102 - DNA, 180 bp, Synthetic construct
  source 1..180: mol_type = other DNA
ttcaatgtactgatggtccataaacggagtcacactggcgagcgcctgctccaatgtaagatctgcgggttcacgtgtcgggagaagggcaacctcctccggcatatcaagctgcacacgggtgtaaaaacggtaaagtgccatctctgcaattacgcctgtcagagaagagatgctttg

SEQ ID NO: 103 - DNA, 180 bp, Synthetic construct
  source 1..180: mol_type = other DNA
ttcaacgttcttatggtgcataagcgcagccacacaggagagaggccactgttttgcccaatttgtggcttcacctgtagacaaaagggaaaccttctccggcatatcaaccttcacaccggggagaagttgtttaagtatcatttgtgcaactatgcttgccagcggcgagacgcattg

SEQ ID NO: 104 - DNA, 180 bp, Synthetic construct
  source 1..180: mol_type = other DNA
ttcaatgtactgatggtccataaacggagtcacactggcgagcgcccgctcatctgtccggtctgcgggttcacgtgtcggcagaagggcaacctcctccggcatatcaatctgcacccgggtgaaaaactgtttaagtaccatctctgcaattacgcctgtcagagaagagatgctttg

SEQ ID NO: 105 - DNA, 180 bp, Synthetic construct
  source 1..180: mol_type = other DNA
ttcaatgtactgatggtccataaacggagtcacactggcgagcgcctcctcatctgtccggtctgcgggttcacgtgtcggcagaagggcaacctcctccggcatatcaatctgcacccgggtgaaaaactgtttaagtaccatctctgcaattacgcctgtcagagaagagatgctttg

SEQ ID NO: 106 - DNA, 180 bp, Synthetic construct
  source 1..180: mol_type = other DNA
ttcaatgtactgatggtccataaacggagtcacactggcgagctcctgctcttctgtccgatctgcgggttcacgtgtcggcagaagggcaacctcctccggcatatcaatctgcacacgggtgaaaaactgtttaagtaccatctctgcaattacgcctgtcagagaagagatgctttg

SEQ ID NO: 107 - DNA, 180 bp, Synthetic construct
  source 1..180: mol_type = other DNA
ttcaatgtactgatggtccataaacggagtcacactggcgagctcctgctcttctgtccgatctgcgggttcacgtgtcggcagaagggcaacctcctccggcatatcaatctgcacacgggtgaaaaactgtttaagtaccatctctacaattacgcctgtcagagaagagatgctttg

SEQ ID NO: 108 - DNA, 180 bp, Synthetic construct
  source 1..180: mol_type = other DNA
tttaacgtgctcatggtgcataagcgaagtcatacgggggaacggctgctgttctgtccgatttgtgggtttacgtgcaggcaaaaaggtaacttgctccgccacataaatctgcacactggagaaaaactctttaagtatcatctctataactatgcttgccaacgaagggatgctttg

SEQ ID NO: 109 - DNA, 180 bp, Synthetic construct
  source 1..180: mol_type = other DNA
ttcaatgtgctgatggtccataaacgaagtcacacaggtgagctgccgttgttttgtcccatttgcggctttacgtgccggcaaaaagggaatttgctccgccacattaaccttcacaccggagaaaagctctttaagtaccatctctataattacgcctgtcagcgccgcgacgctttg

SEQ ID NO: 110 - DNA, 180 bp, Synthetic construct
  source 1..180: mol_type = other DNA
tttaacgtattgatggttcacaaacggtcacacactggagagttgctgctccagtgtccgatatgtggcttcacttgccgccagaaaggtaacttgttgcggcatatcaatctccacacaggcgaaaagctgttcaagtaccacctgtataactatgcatgtcagagaagagatgccctg

SEQ ID NO: 111 - DNA, 180 bp, Synthetic construct
  source 1..180: mol_type = other DNA
ttcaatgtattgatggtccacaagaggagccacactggagagcttttgctgttttgcgaaatttgcggctttacatgcagacaaaaaggaaacctccttcgccacatcaaccttcacactggagagaagctcttcaagtaccacttgtataactacgcatgtcaacgccgcgacgctctt

SEQ ID NO: 112 - DNA, 180 bp, Synthetic construct
  source 1..180: mol_type = other DNA
ttcaacgtactcatggtgcataagagatcacatacgggtgagctcttgctcttttgtcccatttgcggttttacctgtcggcaaaagggtaatctcctcaggcacatcaagttgcacacgggagagaagctgttcaaatatcatctttacaactacgcctgtcaaagaagggacgctctc

SEQ ID NO: 113 - DNA, 180 bp, Synthetic construct
  source 1..180: mol_type = other DNA
tttaatgttcttatggtccacaagagatcacatacaggcgaactcctgctgttttgccctatttgcggattcacatgcaggcagaagggaaacctgctccggcacatcaacttgcatacaggggagaagccatttaagtaccacctttacaactacgcttgccaacgccgggacgcattg

SEQ ID NO: 114 - DNA, 180 bp, Synthetic construct
  source 1..180: mol_type = other DNA
ttcaacgtgctgatggtccacaagcgatctcacactggagagcttcttcttttttgccctatatgtgggtttacttgcagacaaaaggggaacttgctcagacatatcaatctccatacaggagaaaaactgtttaaatgtcacctctataactatgcgtgtcagcggcgggatgcactc

SEQ ID NO: 115 - DNA, 180 bp, Synthetic construct
  source 1..180: mol_type = other DNA
ttcaatgtgctgatggttcacaaacgcagtcacactggggaacttttgcttttctgccctatatgtggttttacatgccgccagaagggaaacttgctccggcatatcaatctccataccggggaaaagctctttaagtgccacttgtgtaactacgcatgtcagcggagggacgcgctg

SEQ ID NO: 116 - DNA, 138 bp, Synthetic construct
  source 1..138: mol_type = other DNA
ctcctgttgttttgcccgatctgcggtttcacttgtcggcagaaaggcaatcttctcaggcacatcaacctccatacaggagagaaactcttcaagtaccatctgtataactatgcctgtcaacgacgagatgccctg

SEQ ID NO: 117 - DNA, 150 bp, Synthetic construct
  source 1..150: mol_type = other DNA
ttcaatgtactgatggtccataaacggagtcacactggcgagctcctgctcttctgtccgatctgcgggttcacgtgtcggcagaagggcaacctcctccggcatatcaatctgcacacgggtgaaaaactgtttaagtaccatctctac

SEQ ID NO: 118 - DNA, 114 bp, Synthetic construct
  source 1..114: mol_type = other DNA
ctcctgttgttttgcccgatctgcggtttcacttgtcggcagaaaggcaatcttctcaggcacatcaacctccatacaggagagaaactcttcaagtaccatctgtataactat

SEQ ID NO: 119 - DNA, 108 bp, Synthetic construct
  source 1..108: mol_type = other DNA
ctcctgctcttttgccccatctgcggattcacctgtaggcagaaagggaatctccttcggcacatcaacttgcatacaggtgagaaattgttcaagtatcatctgtac

SEQ ID NO: 120 - DNA, 99 bp, Synthetic construct
  source 1..99: mol_type = other DNA
cttcttctcttttgccctatttgtgggttcacttgcagacagaagggaaacctcctgcggcatatcaatctccataccggagagaaactcttcaagtat

SEQ ID NO: 121 - DNA, 90 bp, Synthetic construct
  source 1..90: mol_type = other DNA
cttcttctgttctgccccatctgtgggtttacttgtaggcaaaaggggaatctgttgcggcatatcaaccttcacactggagagaaactc

SEQ ID NO: 122 - DNA, 75 bp, Synthetic construct
  source 1..75: mol_type = other DNA
cttctgttgttttgtccaatctgcgggttcacttgccgccagaaaggcaacctgcttcggcatataaatttgcat

SEQ ID NO: 123 - DNA, 72 bp, Synthetic construct
  source 1..72: mol_type = other DNA
ctcctgttttgccctatttgtggatttacatgcaggcaaaaaggtaacctcctgagacatataaacctgcac

SEQ ID NO: 124 - protein, 60 aa, Synthetic construct
FNVLMVHKRSHTGELLLFCPICGFTCRQKGNLLRHINLHPGEKLFRYHLYNYACQRRDAL

SEQ ID NO: 125 - protein, 60 aa, Synthetic construct
FNVLMVHKRSHTGELLLFCPICGFTCRQKGTLLRHINLHPGEKLFRYHLYNYACQRRDAL

SEQ ID NO: 126 - DNA, 180 bp, synthetic construct
  source 1..180: mol_type = other DNA
ttcaatgtgcttatggtccacaaacgctcccacactggcgaacttctgttgttttgcccaatatgcggtttcacatgcaggcaaaaaggaaatctgctgcgccatattaaccttcaccccggtgaaaaactcttccggtatcacctctataactacgcatgtcaaagacgagatgctctg

SEQ ID NO: 127 - DNA, 180 bp, synthetic construct
  source 1..180: mol_type = other DNA
ttcaatgtactcatggtacacaagaggagtcatacaggggaactcctcttgttctgtccaatctgcggctttacttgtcgccaaaaaggtacactcctcaggcacatcaaccttcatccaggggagaaactgtttaggtatcatctctataactatgcctgccagaggcgggatgctctg

SEQ ID NO: 128 - DNA, 180 bp, synthetic construct
  source 1..180: mol_type = other DNA
ttcaatgtactgatggtccataaacggagtcacactggcgagctcctgctcttttgtcctatttgcgggttcacgtgtcggcagaagggcaacctcctccggcatatcaatctgcacccgggtgaaaaactgtttaggtaccatctctacaattacgcctgtcagagaagagatgctttg

SEQ ID NO: 129 - DNA, 180 bp, synthetic construct
  source 1..180: mol_type = other DNA
ttcaatgtactgatggtccataaacggagtcacactggcgagctcctgctcttttgtcctatttgcgggttcacgtgtcggcagaagggcaccctcctccggcatatcaatctgcacccgggtgaaaaactgtttaggtaccatctctacaattacgcctgtcagagaagagatgctttg

SEQ ID NO: 130 - protein, 11 aa, Synthetic construct
RPQENKTPKCC

SEQ ID NO: 131 - protein, 11 aa, Synthetic construct
LLFPNNTLKYY

SEQ ID NO: 132 - protein, 11 aa, Synthetic construct
LLFPNNPLRYY

SEQ ID NO: 133 - protein, 11 aa, Synthetic construct
LLFPTNPLRYY

SEQ ID NO: 134 - protein, 23 aa, Synthetic construct
FQCNQCGASFTQKGNLLRHIKLH

SEQ ID NO: 135 - protein, 57 aa, Synthetic construct
LQCEICGFTCRQKGNLLRHIKLHTGEKPFKCHLCNTACQRRDALTGHLRTHSVIKEE